SEQUENCE LISTING (128 sequences)

SEQ ID NO: 1 - DNA, 555 bp, Artificial Sequence
  xylanase modified at position 79
gcttcgacagactactggcaaaattggactgatggtggtgggacagtaaatgctaccaatggatctgatggcaattacagcgtttcatggtcaaattgcgggaattttgttgttggtaaaggctggactaccggatcagcaactagggtaataaactataatgccggagccttttcgccgtccggtaatggatatttggctctttatgggtggacgagaaattcactcatagcttattacgtcgttgatagctgggggacttatagacctactggaacttataaaggcactgtgactagtgatggagggacttatgacatatacacgactacacgaaccaacgcaccttccattgacggcaataatacaactttcacccagttctggagtgttaggcagtcgaagagaccgattggtaccaacaataccatcacctttagcaaccatgttaacgcctggaagagtaaaggaatgaatttggggagtagttggtcttatcaggtattagcaacagagggctatcaaagtagtgggtactctaacgtaacggtctgg

SEQ ID NO: 2 - protein, 185 aa, Artificial Sequence
  Synthetic Construct
ASTDYWQNWTDGGGTVNATNGSDGNYSVSWSNCGNFVVGKGWTTGSATRVINYNAGAFSPSGNGYLALYGWTRNSLIAYYVVDSWGTYRPTGTYKGTVTSDGGTYDIYTTTRTNAPSIDGNNTTFTQFWSVRQSKRPIGTNNTITFSNHVNAWKSKGMNLGSSWSYQVLATEGYQSSGYSNVTVW

SEQ ID NO: 3 - protein, 185 aa, Artificial Sequence
  environmental sample
ASTDYWQNWTDGGGTVNATNGSDGNYSVSWSNCGNFVVGKGWTTGSATRVINYNAGAFSPSGNGYLALYGWTRNSLIEYYVVDSWGTYRPTGTYKGTVTSDGGTYDIYTTTRTNAPSIDGNNTTFTQFWSVRQSKRPIGTNNTITFSNHVNAWKSKGMNLGSSWSYQVLATEGYQSSGYSNVTVW

SEQ ID NO: 4 - protein, 211 aa, Aeromonas punctata ME-1
MFKFGKKLMTVVLAASMSFGVFAATSSAATDYWQNWTDGGGTVNAVNGSGGNYSVSWQNTGNFVVGKGWTYGTPNRVVNYNAGVFAPSGNGYLTFYGWTRNALIEYYVVDSWGTYRPTGTYKGTVNSDGGTYDIYTTMRYNAPSIDGTQTFPQYWSVRQSKRPTGVNSTITFSNHVNAWPSKGMYLGNSWSYQVMATEGYQSSGNANVTVW

SEQ ID NO: 5 - protein, 227 aa, Ascochyta pisi
MVSFTSIFTAAVAATGALAVPVTDLATRSLGALTARAGTPSSQGTHNGCFYSWWTDGGAQATYTNGAGGSYSVNWKTGGNLVGGKGWNPGAARTITYSGTYSPSGNSYLAVYGWTRNPLIEYYVVENFGTYDPSSQATVKGSVTADGSSYKIAQTQRTNQPSIDGTQTFQQYWSVRQNKRSSGSVNMKTHFDAWAAKGMKLGTHNYQIVATEGYFSSGSAQITVNCA

SEQ ID NO: 6 - protein, 120 aa, Ascochyta rabiei
ARTITYSGTYSPSGNSYLAVYGWTRNPLIEYYVVENFGTYDPSSQATVKGSVTADGSSYKIAQTQRTNQPSIDGTQTFQQYWSVRQSKRSSGSVNMKTHFDAWAAKGMKLGTHYYQIVAV

SEQ ID NO: 7 - protein, 231 aa, Aspergillus aculeatus
  MISC_FEATURE (1)..(231): xylanase
MARLSQFLLACALAVKAFAAPAAEPVEERGPNFFSALAGRSTGSSTGYSNGYYYSFWTDGASGDVEYSNGAGGSYSVTWSSASNFVGGKGWNPGSAHDITYSGSWTSTGNSNSYLSVYGWTTGPLVEYYILEDYGEYNPGSAGTYKGSVYSDGSTYNIYTATRTNAPSIQGTATFTQYWSIRQTKRVGGTVTTANHFNAWAKLGMNLGTHNYQIVATEGYYSSGSASITVA

SEQ ID NO: 8 - protein, 211 aa, Aspergillus awamorii
  MISC_FEATURE (1)..(211): xylanase
MKVTAAFAGLLVTAFAAPVPEPVLVSRSAGINYVQNYNGNLGDFTYDESAGTFSMYWEDGVSSDFVVGLGWTTGSSNAITYSAEYSASGSSSYLAVYGWVNYPQAEYYIVEDYGDYNPCSSATSLGTVYSDGSTYQVCTDTRTNEPSITGTSTFTQYFSVRESTRTSGTVTVANHFNFWAQHGFGNSDFNYQVMAVEAWSGAGSASVTISS

SEQ ID NO: 9 - protein, 225 aa, Aspergillus cf. niger
  MISC_FEATURE (1)..(225): xylanase
MLTKNLLLCFAAAKAVLAVPHDSVVERSDALHKLSERSTPSSTGENNGFYYSFWTDGGGDVTYTNGDAGSYTVEWSNVGNFVGGKGWNPGSAQDITYSGTFTPSGNGYLSVYGWTTDPLIEYYIVESYGDYNPGSGGTYKGTVTSDGSVYDIYTATRTNAASIQGTATFTQYWSVRQNKRVGGTVTTSNHFNAWAKLGMNLGTHNYQIVATEGYQSSGSSSITVQ

SEQ ID NO: 10 - protein, 211 aa, Aspergillus kawachii
  MISC_FEATURE (1)..(211): xylanase
MKVTAASAGLLGHAFAAPVPQPVLVSRSAGINYVQNYNGNLADFTYDESAGTFSMYWEDGVSSDFVVGLGWTTGSSNAISYSAEYSASGSSSYLAVYGWVNYPQAEYYIVEDYGDYNPCSSATSLGTVYSDGSTYQVCTDTRTNEPSITGTSTFTQYFSVRESTRTSGTVTVANHFNFWAQHGFGNSDFNYQVMAVEAWSGAGSASVTISS

SEQ ID NO: 11 - protein, 225 aa, Aspergillus kawachii
  MISC_FEATURE (1)..(225): xylanase
MLTKNLLLCFAAAKAVLAVPHDSVVERSDALHKLSERSTPSSTGENNGYYYSFWTDGGGDVTYTNGNAGSYSVEWSNVGNFVGGKGWNPGSAKDITYSGNFTPSGNGYLSVYGWTTDPLIEYYIVESYGDYNPGSGGTTRGNVSSDGSVYDIYTATRTNAPSIQGTATFSQYWSVRQNKRVGGTVTTSNHFNAWAKLGMNLGTHNYQILATEGYQSSGSSSITIQ

SEQ ID NO: 12 - protein, 221 aa, Aspergillus nidulans
  MISC_FEATURE (1)..(221): xylanase
MVSFSSLLLACSAVTAFAAPSDQSIAERSLSERSTPSSTGTSGGYYYSFWTDGGGDVTYTNGDGGSYTVEWTKVGNFVGGKGWNPGSSQTISYSGSFIPSGNGYLSVYGWTQNPLIEYYIVESYGDYNPGTAGTHQGTLESDGSTYDIYTATRENAPSIEGTATFTQFWSVRQSKRTSGSVTTQNHFDAWSQLGMTLGTHNYQIVAVEGYQSSGSASITVS

SEQ ID NO: 13 - protein, 211 aa, Aspergillus niger
  MISC_FEATURE (1)..(211): xylanase
MKVTAAFAGLLVTAFAAPVPEPVLVSRSAGINYVQNYNGNLGDFTYDESAGTFSMYWEDGVSSDFVVGLGWTTGSSKAITYSAEYSASGSSSYLAVYGWVNYPQAEYYIVEDYGDYNPCSSATSLGTVYSDGSTYQVCTDTRTNEPSITGTSTFTQYFSVRESTRTSGTVTVANHFNFWAQHGFGNSDFNYQVMAVEAWSGAGSASVTISS

SEQ ID NO: 14 - protein, 211 aa, Aspergillus niger
  MISC_FEATURE (1)..(211): xylanase
MKVTAAFASLLLTAFAAPAPEPVLVSRSAGINYVQNYNGNLGDFTYDESTGTFSMYWEDGVSSDFVVGLGWTTGSSKSITYSAQYSASSSSSYLAVYGWVNSPQAEYYIVEDYGDYNPCSSATSLGTVYSDGSTYQVCTDTRRTRPSITGTSTFTQYFSVRESTRTSGTVTIANHFNFWAQHGFGNSNFNYQVMAVEAWNGVGSASVTISS

SEQ ID NO: 15 - protein, 225 aa, Aspergillus niger; identical to SEQ ID NO: 9

SEQ ID NO: 16 - protein, 225 aa, Aspergillus niger
  MISC_FEATURE (1)..(225): xylanase
MLTKNLLLCFAAAKAALAVPHDSVAQRSDALHMLSERSTPSSTGENNGFYYSFWTDGGGDVTYTNGDAGAYTVEWSNVGNFVGGKGWNPGSAQDITYSGTFTPSGNGYLSVYGWTTDPLIEYYIVESYGDYNPGSGGTYKGTVTSDGSVYDIYTATRTNAASIQGTATFTQYWSVRQNKRVGGTVTTSNHFNAWAKLGMNLGTHNYQIVATEGYQSSGSSSITVQ

SEQ ID NO: 17 - protein, 221 aa, Aspergillus oryzae
  MISC_FEATURE (1)..(221): xylanase
MVSFSSLLLAVSAVSGALAAPGDSTLVELAKRAITSSETGTNNGYYYSFWTNGGGDVEYTNGNGGQYSVKWTNCDNFVAGKGWNPGSAKTVTYSGEWESNSNSYVSLYGWTQNPLVEYYIVDKYGDYDPSTGATELGTVESDGGTYKIYKTTRENAPSIEGTSTFNQYWSVRQSGRVGGTITAQNHFDAWANVGLQLGTHNYMILATEGYKSSGSATITVE

SEQ ID NO: 18 - protein, 232 aa, Aspergillus oryzae
  MISC_FEATURE (1)..(232): xylanase
MVSFSSILLACSAAIGALATPIEPLADHPNEAFNETAFNDLVGRSTPSSTGYNNGYYYSFWTDGGGDVTYTNGNGGSYSVQWSNVGNFVGGKGWNPGSSRAITYSGSFNPSGNGYLAVYGWTTDPLIEYYIVESYGTYNPGSGGTYKGQVTSDGGTYNIYTSVRTNAPSIIGTATFTQFWSVRTSKRVGGTVTTGNHFNAWAKYGLTLGTHNYQIVATEGYQSSGSSAITVY

SEQ ID NO: 19 - protein, 211 aa, Aspergillus tubingensis
  MISC_FEATURE (1)..(211): xylanase
MKVTAAFAGLLVTAFAAPAPEPDLVSRSAGINYVQNYNGNLGDFTYDESAGTFSMYWEDGVSSDFVVGLGWTTGSSNAITYSAEYSASGSASYLAVYGWVNYPQAEYYIVEDYGDYNPCSSATSLGTVYSDGSTYQVCTDTRTNEPSITGTSTFTQYFSVRESTRTSGTVTVANHFNFWAHHGFGNSDFNYQVVAVEAWSGAGSASVTISS

SEQ ID NO: 20 - protein, 221 aa, Aureobasidium pullulans var. melanigenum
  MISC_FEATURE (1)..(221): xylanase
MKFFATIAALVVAAVAAPVAEADAEASSPMLIERAGPGGINYVQNYNGNLGQFTYNENAGTYSMYWTNGVSGDFVVGLGWSTGAARSITYSSSYTASGGSYLSVYGWINSPQAEYYIVESYGSYNPCGAGQSGVTQLGTVVSDGATYTVCTDERVNEPSITGTSTFKQYWSVRQTKRTSGTVTTGNHFAYWAKYGFGNSYNFQVMAVEAFSGTGSASVTVS

SEQ ID NO: 21 - protein, 221 aa, Aureobasidium pullulans Y-2311-1
  MISC_FEATURE (1)..(221): xylanase
MKFFATIAALVVGAVAAPVAEAEAEASSPMLIERAGPGGINYVQNYNGNLGQFTYNENAGTYSMYWNNGVNGDFVVGLGWSTGAARSITYSSNYQASGGSYLSVYGWINSPQAEYYIVESYGSYNPCGAGQSGVTQLGTVCSDGATYTVYTDTRTNQPSITGTSTFKQYWSVRQTKRTSGTVTTGNHFAYWAKYGFGNSYNFQVMPVEAFSGTGSASVTVS

SEQ ID NO: 22 - protein, 221 aa, Bacillus agaradhaerens AC13
  MISC_FEATURE (1)..(221): xylanase
QIVTDNSIGNHDGYDYEFWKDSGGSGTMILNHGGTFSAQWNNVNNILFRKGKKFNETQTHQQVGNMSINYGANFQPNGNAYLCVYGWTVDPLVEYYIVDSWGNWRPPGATPKGTITVDGGTYDIYETLRVNQPSIKGIATFKQYWSVRRSKRTSGTISVSNHFRAWENLGMNMGKMYEVALTVEGYQSSGSANVYSNTLRINGNPLSTISNDESITLDKNN

SEQ ID NO: 23 - protein, 213 aa, Bacillus circulans
  MISC_FEATURE (1)..(213): xylanase
MFKFKKNFLVGLSAALMRIILFSATASAASTDYWQNWTDGGGIVNAVNGSGGNYSVNWSNTGNFVVGKGWTTGSPFRTINYNAGVWAPNGNGYLTLYGWPRSPLIEYYVVDSWGTYRPTGTYKGTVKSDGGTYDIYTTTRYNAPSIDGDRTTFTQYWSVRQTKRPTGSNATITFSNHVNAWKSHGMNLGSNWAYQVMATGGYQSSGSSNVTVW

SEQ ID NO: 24 - protein, 210 aa, Bacillus firmus
  MISC_FEATURE (1)..(210): xylanase
MFKFVTKVLTVVIAATISFCLSAVPASANTYWQYWTDGGGTVNATNGPGGNYSVTWRDTGNFVVGKGWEIGSPNRTIHYNAGVWEPSGNGYLTLYGWTRNQLIEYYVVDNWGTYRPTGTHRGTVVSDGGTYDIYTTMRYNAPSIDGTQTFQQFWSVRQSKRPTGNNVSVTFSNHVNAWRNAGMNLGSSWSYQVLATEGYQSSGRSNVTVW

SEQ ID NO: 25 - protein, 210 aa, Bacillus firmus K-1
  MISC_FEATURE (1)..(210): xylanase
MFKFVTKVLTVVIAATISFCLSAVPASANTYWQYWTDGGGTVNATNGPGGNYSVTWRDTGNFVVGKGWEIGSPNRTIHYNAGVWEPSGNGYLTLYGWTRNQLIEYYVVDNWGTYRPTGTHRGTVVSDGGTYDIYTTMRYNAPSIDGTQTFQQFWSVRQSKRPTGNNVSITFSNHVNAWRNAGMNLGSSWSYQVLATEGYQSSGRSNVTVW

SEQ ID NO: 26 - protein, 210 aa, Bacillus halodurans C-125; identical to SEQ ID NO: 25
  MISC_FEATURE (1)..(210): xylanase

SEQ ID NO: 27 - protein, 228 aa, Bacillus pumilus
  MISC_FEATURE (1)..(228): xylanase
MNLRKLRLLFVMCIGLTLILTAVPAHARTITNNEMGNHSGYDYELWKDYGNTSMTLNNGGAFSAGWNNIGNALFRKGKKFDSTRTHHQLGNISINYNASFNPGGNSYLCVYGWTQSPLAEYYIVDSWGTYRPTGAYKGSFYADGGTYDIYETTRVNQPSIIGIATFKQYWSVRQTKRTSGTVSVSAHFRKWESLGMPMGKMYETAFTVEGYQSSGSANVMTNQLFIGN

SEQ ID NO: 28 - protein, 228 aa, Bacillus pumilus HB030
  MISC_FEATURE (1)..(228): xylanase
MNLRKLRLLFVMCIGLTLILTAVPAHARTITNNEMGNQSGYDYELWKDYGNTSMTLNNGGAFSSGWNNIGNALFRKGKKFDSTRTHHQLGNISINYNASFNPGGNSYLCVYVWTQSPLAEYYIVDSWGTYRPTGAYKGSFYADGGTYDIYETTLVNQPSIIGIATFKQYLSVRQTKRTSGTVSVSAHFRKWESLGKPMGKMYETAFTVEGYQSSGSANVMTNQLFIGN

SEQ ID NO: 29 - protein, 213 aa, Bacillus sp.
  MISC_FEATURE (1)..(213): xylanase
MFKFKKKFLVGLTAAFMSISMFSATASAAGTDYWQNWTDGGGTVNAVNGSGGNYSVNWSNTGNFVVGKGWTTGSPFRTINYNAGVWAPNGNGYLTLYGWTRAPLIEYYVVDSWGTYRPTGTYKGTVKSDGGTYDIYTTTRYNAPSIDGDNTTFTQYWSVRQSKRPTGSNAAITFSNHVNAWKSHGMNLGSNWAYQVLATEGYKSSGSSNVTVW

SEQ ID NO: 30 - protein, 213 aa, Bacillus sp. YA-14
  MISC_FEATURE (1)..(213): xylanase
MFKFKKNFLVGLSAALMSISLFSATASAASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQTKRPTGSNATITFSNHVNAWKSHGMNLGSNWAYQVLATEGYQSSGSSNVTVW

SEQ ID NO: 31 - protein, 355 aa, Bacillus sp. YA-335
  MISC_FEATURE (1)..(355): xylanase
MKQVKIMFLMTMVLGIGLLFFSENAEAAITSNEIGTHDGYDYEFWKDSGGSGSMTLNSGGTFSAQWSNVNNILFRKGKKFDETQTHQQIGNMSINYGATYNPNGNSYLTVYGWTVDPLVEFYIVDSWGTWRPPGGTPKGTINVDGGTYQIYETTRYNQPSIKGTATFQQYWSVRTSKRTSGTISVSEHFRAWESLGMNMGNMYEVALTVEGYQSSGSANVYSNTLTIGGQSGGEQATRVEAESMTKGGPYTSNITSPFNGVALYANGDNVSFNHSFTKANSSFSLRGASNNSNXMARVDLRIGGQNRGTFYFGDHYPAVYTINNVNHGTGNQLVELIVTADDGTWDAYLDYLEIR

SEQ ID NO: 32 - protein, 205 aa, Bacillus subtilis B230
  MISC_FEATURE (1)..(205): xylanase
ATTITSNQTGTHDGYDYELWKDSGNTSMTLNSGGAFSAQWSNIGNALFRKGKKFDSTKTHSQLGNISINYNATFNPGGNSYLCVYGWTKDPLTEYYIVDNWGTYRPTGTPKGTFTVDGGTYDIYETTRINQPSIIGIATFKQYWSVRQTKRTSGTVSVSEHFKKWESLGMPMGKMYETALTVEGYQSNGSANVTANVLTIGGKPL

SEQ ID NO: 33 - protein, 213 aa, Bacillus subtilis subsp. subtilis str. 168
  MISC_FEATURE (1)..(213): xylanase
MFKFKKNFLVGLSAALMSISLFSATASAASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 34 - protein, 361 aa, Caldicellulosiruptor sp. Rt69B.1
  MISC_FEATURE (1)..(361): xylanase
MCVVLANPFYAQAAMTFTSNATGTYDGYYYELWKDTGNTTMTVDTGGRFSCQWSNINNALFRTGKKFSTAWNQLGTVKITYSATYNPNGNSYLCIYGWSRNPLVEFYIVESWGSWRPPGATSLGTVTIDGATYDIYKTTRVNQPSIEGTRTFDQYWSVRTSKRTSGTVTVTDHFKAWAAKGLNLGTIDQITLCVEGYQSSGSANITQNTFTIGGSSSGSSNGSNNGSNDGSNGGTNAGISTASRIECESMSLSGPYVSRITYPFNGIALYANGDRATANVNFSASRNYTFKLRGCGNNNNLASVDLLIDGKKVGSFYYKGTYPWEASINNVYVSAGTHRVELVLSADNGTWDVYADYLLIQ

SEQ ID NO: 35 - protein, 644 aa, Cellulomonas fimi
  MISC_FEATURE (1)..(644): xylanase
MSDSFEATRTTRRRRPLQALTGLLAAGALVAGALAAASPAAAAVTSNTTGTHDGYFYSFWTDSPGSVSMDLNSGGGYTRWSNTGNFVAGKGWSTGGRKTVSYSGQFNPSRNAYLTLYGWTQSPLVEYYIVDSWGTYRPTGTFMGTVTSDGGTYDIYRTQRVNKPSIEGDSSTFYQYWSVRQQKRTGGTITSGNHFDAWASKGMNLGRHNYMIMATEGYQSSGSSSITVSEGSGGGGGGDTGGGGGSTGCSVTATRAEEWSDRFNVTYSVSGSSAWTVNLALNGSQTIQASWNANVTGSGSTRTVTPNGSGNTFGVTVMKNGSSTTPAATCAGSGGGTATPTPTPTPTPTPQSCSAGYVGLTFDDGPNTGTTNQILSTLTQYGATATVFPTGQNAQGNPSLMQAYKNAGVQIGNHSWDHPHLVNMSQSDMQSQLTRTQQAIQQTAGVTPTLFRPPYGESNATLRQVESSLGLREIIWDVDSQDWNNASASQIRQAASRLTNGQIILMHDWPAATVQALPGILQDLRSRNLCTGHISSSTGRAVAPSSAGGGGGGGGGTGSCSVSAVRGEEWADRFNVTYSVSGSSSWVVTLGLNGGQSVQSSWNAALTGSSGTVTARPNGSGNSFGVTFYKNGSSATPGATCATG

SEQ ID NO: 36 - protein, 335 aa, Cellulomonas pachnodae
  MISC_FEATURE (1)..(335): xylanase
MTRTISRAAHRPPAGGRIARALAAAGATVAMVIAGVAAAQPAAAVDSNSTGSSGGYFYSFWTDAPGTVSMNLGSGGNYSTSWSNTGNFVAGKGWSTGSARTISYSGTFNPSGNAYLAVYGWSHDPLVEYYIVDSWGTYRPTGTFMGTVNSDGGTYDIYKTTRTNAPSIEGTATFTQYWSVRQSKRVGGTITTANHFNAWASHGMNLGRHDYQILATEGYQSSGSSNITIGSTSGGGGSGGGTTTPPTNNGCTVQVTKGDEWGDRFNVSFTVSGSSSWKVAIALSGGQSLQNSWSANVTGSTGTLTATPNGSGNSFGITVYKNGSSTLPTATCSTT

SEQ ID NO: 37 - protein, 660 aa, Cellvibrio japonicus
  MISC_FEATURE (1)..(660): xylanase
KLPTLGKCVVRTLMGAVALGAISVNAQTLSSNSTGTNNGFYYTFWKDSGDASMTLLSGGRYQSSWGNSTNNWVGGKGWNPGNNSRVISYSGSYGVDSSQNSYLALYGWTRSPLIEYYVIESYGSYNPASCSGGTDYGSFQSDGATYNVRRCQRVNQPSIDGTQTFYQYFSVRNPKKGFGNISGTITFANHVNFWASKGLNLGNHNYQVLATEGYQSRGSSDITVSEGTSGGGTSSVGGASSSVNSSTGGGSSGGITVRARGANGSEHINLRVGGAVVANWTLGTSFQNYLYSGNASGDIQVQFDNDASGRDVVVDYIIVNGETRQAEDMEHNSAVYANGRCGGGSYSENMHCNGEIGFGYTYDCFSGNCSGGNGGSNSSAGNSSSGNTGGGGSNCSGYVGITFDDGPNSNTATLVNLLRQNNLTPVTWFNQGNNVASNAHLMSQQLSVGEVHNHSYTHPHMTSWTYQQVYDELNRTNQAIQNAGAPKPTLFRPPYGELNSTIQQAAQALGLRVVTWDVDSQDWNGASAAAIANAANQLQNGQVILMHDGSYTNTNSAIAQIATNLRAKGLCPGRIDPNTGRAVAPSSSGGSSSVALSSSSRSSSSAGGNTGGNCQCNWWGTFYPLCQTQTSGWGWENSRSCISTSTCNSQGTGGGGVVCN

SEQ ID NO: 38 - protein, 656 aa, Cellvibrio mixtus
  MISC_FEATURE (1)..(656): xylanase
MKFPLIGKSTLAALFCSALLGVNNTQAQTLTNNATGTHNGFYYTFWKDSGDASMGLQAGGRYTSQWSNGTNNWVGGKGWNPGGPKVVTYSGSYNVDNSQNSYLALYGWTRSPLIEYYVIESYGSYNPASCSGGTDYGSFQSDGATYNVRRCQRVQQPSIDGTQTFYQYFSVRSPKKGFGQISGTITTANHFNFWASKGLNLGNHDYMVLATEGYQSRGSSDITVSEGTGGTTSSSVPSNGSGSSSSVSANTGGVLVRARGVAGGEYINLRIGGATVASWNLTTSFQDLNYTGTASGDIQVQFDNDGGSRDVVVDYIRVNGETRQAEDMSYNTAFYTNGSCGGGGNSELMHCNGAIGFGYTYDCFSGNCSGGSTGGGTNSSVASSAGSTASCAGYVGITFDDGPGANTTTLVNLLKQNNLTPVTWFVQGNYVAANSNLMSQLLSVGEVQNHSYTHPHLINLGYQQIYDELNRTNQAIQNAGAPKPTLFRPPYGEVNANVNQAAQALGLRVITWNVDSQDWNGASATAIANAANQLQNGQVILMHDANYNNTNAAIAQIASNLRAKGLCPGRIDAATGRAVAPAGAVASSSRSSSSTPIVASSSSVATGNACQCNWWGTRYPLCTNTASGWGWENNTSCITTSTCNSQGAGGGGVVCN

SEQ ID NO: 39 - protein, 219 aa, Chaetomium gracile
  MISC_FEATURE (1)..(219): xylanase
MVSFKALLLGAAGALAFPFNVTQMNELVARAGTPSGTGTNNGYFYSFWTDGGGTVNYQNGAGGSYSVQWQNCGNFVGGKGWNPGAARTINFSGTFSPQGNGYLAIYGWTQNPLVEYYIVESFGTYDPSSQASKFGTIQQDGSTYTIAKTTRVNQPSIEGTSTFDQFWSVRQNHRSSGSVNVAAHFNAWAQAGLKLGSHNYQIVATEGYQSSGSSSITVS

SEQ ID NO: 40 - protein, 241 aa, Chaetomium gracile
  MISC_FEATURE (1)..(241): xylanase
MVNFSSLFLAASAAVVAVAAPGELPGMHKRQTLTSSQTGTNNGYYYSFWTDGQGNVQYTNEAGGQYSVTWSGNGNWVGGKGWNPGSARTINYTANYNPNGNSYLAVYGWTRNPLIEYYVVENFGTYNPSTGATRLGSVTTDGSCYDIYRTQRVNQPSIEGTSTFYQFWSVRQNKRSGGSVNMAAHFNAWAAAGLQLGTHDYQIVATEGYYSSGSATVNVGASSDGSTGGGSTGGGSTNVSF

SEQ ID NO: 41 - protein, 261 aa, Chaetomium thermophilum
  MISC_FEATURE (1)..(261): xylanase
MVNFSTLFLAASTAALAAAAPSIEKRQTLTSSATGTHNGYYYSFWTDGQGNIRFNLESGGQYSVTWSGNGNWVGGKGWNPGTDNRVINYTADYRPNGNSYLAVYGWTRNPLIEYYVVESFGTYDPSTGATRMGSVTTDGGTYNIYRTQRVNAPSIEGTKTFYQYWSVRTSKRTGGTVTMANHFNAWRQAGLQLGSHDYQIVATEGYYSSGSATVNVGGSTTGGNNGGNNGGNNGGNNGGNTGSNVSISRPRKMGSLASTRS

SEQ ID NO: 42 - protein, 231 aa, Chaetomium thermophilum
  MISC_FEATURE (1)..(231): xylanase
MVSLKSLLLTAATALAFPLEAFNATEGFNATSLHELMVRAGTSSGTGTHNGWYYSFWTDGGGTVWYTNGNGGSYSVNWQNCGNFVGGKGWRTGAAATIKYSGNYNPSGNSYLAIYGWTRNPLVEYYIVESYGTYDPSSGAQNLGTFQSDGGTYKIAKSTRYNAPSIEGTKTFTQYWSVRTSKRVGGTVXTVANHFNAWKSKGLNLGSHDYQIVATEGYKSSGSASITVQSG

SEQ ID NO: 43 - protein, 224 aa, Chaetomium thermophilum
  MISC_FEATURE (1)..(224): xylanase
MVKLALLTTSLLTSGALTSPVSNPNRPPSRDISPRQWGGGGYYFQNWSEGGSNVRCVNGPGGQFSATWNSKGGFVCGKGWSAGGARVITYSGTYNATGPGYLAVYGWTRNPLIEYYIIEAHAELSPNEPWTYMGNFSSPEGDYDIYTSWRINKPSIEGTRTFQQFWSVRKEQRVSGTVTTQRHFDEWAKLGMRLGRHDYVVMAVEGYTADGGWGSAGEATITVQ

SEQ ID NO: 44 - protein, 216 aa, Claviceps purpurea
  MISC_FEATURE (1)..(216): xylanase
MFLTSVVSLVVGAISCVSAAPAAASELMQMTPRNSCYGGGLYSSYWADYGNTRYSCGAGGHYDLSWGNGGNVVAGRGWKPASPRAVTYSGSWQCNGNCYLSVYGWTINPLVEYYIVENYGNYNPSAGAQRRGQVTADGSIYDIYISTQHNQPSILGTNTFHQYWSIRRNKRVGGTVSTGVHFNAWRSLGMPLGTYDYMIVATEGFRSSGSASITVS

SEQ ID NO: 45 - protein, 520 aa, Clostridium cellulovorans
  MISC_FEATURE (1)..(520): xylanase
MKQKMRIVFSMLMCITILLASSAVGVLAATKTITSNETGNFEGYDYEYWKDNGTGTMTLTGGGTFSCSWSNINNILFRTGKKLGSTKTYKDYGKISINYDCNYQPNGNSYMAVYGWTEDPLVEYYIIDSYGTWNPGSNSTLKGTTTVDGRTYQIYQNSRTGPSIKGNNTTFQQYWSICTSKRTSGTITVSDHFKAWENLGMKMGKMYEVSMVVEGYQSSGKADMTKMDLIMGDTPPVIVDQPTPEKLLGDVDNNGTVNALDLAIYKKFLLGMIPSLPATGDVDQNGRMNAIDFAMIKQHLLGIIKLGKLPTTSEDPNKKLVALTFDDGPSSQTGLVLDKLKKYNAKATFMVIGNKISSADAIMKRIVSEGHEIGNHGWSYDSMANMSASQITQEINQCNSAIKQYTGSDPKFFRPANLATSQTLFQTVKLPFVQGVIAQDWNGASATTAEARAQLVLNGTQDGSIVLMHCTQPGYHPTPEALDIIIPQLQQQGYSFVTLSDLFKLKGKTPQLGTMLNGAF

SEQ ID NO: 46 - protein, 261 aa, Clostridium saccharobutylicum P262
  MISC_FEATURE (1)..(261): xylanase
MLRRKVIFTVLATLVMTSLTIVDNTAFAATNLNTTESTFSKEVLSTQKTYSAFNTQAAPKTITSNEIGVNGGYDYELWKDYGNTSMTLKNGGAFSCQWSNIGNALFRKGKKFNDTQTYKQLGNISVNYDCNYQPYGNSYLCVYGWTSSPLVEYYIVDSWGSWRPPGGTSKGTITVDGGIYDIYETTRINQPSIQGNTTFKQYWSVRRTKRTSGTISVSKHFAAWESKGMPLGKMHETAFNIEGYQSSGKADVNSMSINIGK

SEQ ID NO: 47 - protein, 512 aa, Clostridium stercorarium F-9
  MISC_FEATURE (1)..(512): xylanase
MKRKVKKMAAMATSIIMAIMIILHSIPVLAGRIIYDNETGTHGGYDYELWKDYGNTIMELNDGGTFSCQWSNIGNALFRKGRKFNSDKTYQELGDIVVEYGCDYNPNGNSYLCVYGWTRNPLVEYYIVESWGSWRPPGATPKGTITQWMAGTYEIYETTRVNQPSIDGTATFQQYWSVRTSKRTSGTISVTEHFKQWERMGMRMGKMYEVALTVEGYQSSGYANVYKNEIRIGANPTPAPSQSPIRRDAFSIIEAEEYNSTNSSTLQVIGTPNNGRGIGYIENGNTVTYSNIDFGSGATGFSATVATEVNTSIQIRSDSPTGTLLGTLYVSSTGSWNTYQTVSTNISKITGVHDIVLVFSGPVNVDNFIFSRSSPVPAPGDNTRDAYSIIQAEDYDSSYGPNLQIFSLPGGGSAIGYIENGYSTTYKNIDFGDGATSVTARVATQNATTIQVRLGSPSGTLLGTIYVGSTGSFDTYRDVSATISNTAGVKDIVLVFSGPVNVDWFVFSKSGT

SEQ ID NO: 48 - protein, 683 aa, Clostridium thermocellum F1 / YS
  MISC_FEATURE (1)..(683): xylanase
MRQKLLVTFLILITFTVSLTLFPVNVRADVVITSNQTGTHGGYNFEYWKDTGNGTMVLKDGGAFSCEWSNINNILFRKGFKYDETKRHDQLGYITVTYSCNYQPNGNSYLGVYGWTSNPLVEYYIIESWGTWRPPGATPKGTITVDGGTYEIYETTRVNQPSIKGTATFQQYWSVRTSKRTSGTISVTEHFKAWERLGMKMGKMYEVALVVEGYQSSGKADVTSMTITVGNAPSTSSPPGPTPEPTPRSAFSKIESEEYNSLKSSTIQTIGTSDGGSGIGYIESGDYLVFNKINFGNGANSFKARVASGADTPTNIQLRLGSPTGTLIGTLTVASTGGWNNYEEKSCSITNTTGQHDLYLVFSGPVNIDYFIFDSNGVNPTPTSQPQQGQVLGDLNGDKQVNSTDYTALKRHLLNITRLSGTALANADLNGDGKVDSTDLMILHRYLLGIISSFPRSNPQPSSNPQPSSNPQPTINPNAKLVALTFDDGPDNVLTARVLDKLDKYNVKATFMVVGQRVNDSTAAIIRRMVNSGHEIGNHSWSYSGMANMSPDQIRKSIADTNAVIQKYAGTTPKFFRAPNLETSPTLFNNVDLVFVGGLTANDWIPSTTAEQRAGAVINGVRDGTIILLHDVQPEPHPTPEALDIIIPTLKSRGYEFVTLTELFTLKGVPIDPSVKRMYNSVP

SEQ ID NO: 49 - protein, 457 aa, Clostridium thermocellum F1 / YS
  MISC_FEATURE (1)..(457): xylanase
MKQKLLVTFLILITFTVSLTLFPVNVRADVVITSNQTGTHGGYNFEYWKDTGNGTMVLKDGGAFSCEWSNINNILFRKGFKYDETKTHDQLGYITVTYSCNYQPNGNSYLGVYGWTSNPLVEYYIIESWGTWRPPGATPKGTITVDGGTYEIYETTRVNQPSIKGTATFQQYWSVRTSKRTSGTISVTEHFKAWERLGMKMGKMYEVALVVEGYQSSGKADVTSMTITVGNAPSTSSPPGPTPEPTPRSAFSKIEAEEYNSLKSSTIQTIGTSDGGSGIGYIESGDYLVFNKINFGNGANSFKARVASGADTPTNIQLRLGSPTGTLIGTLTVASTGGWNNYEEKSCSITNTTGQHDLYLVFSGPVNIDYFIFDSKGVNPTPTPTSPPQQDQVLGDLNGDKQVNSTDYTALKRHLLNITRLSGTALANADVNRDGKVDSTDLMMLHRYLLRIISKLG

SEQ ID NO: 50 - protein, 221 aa, Cochliobolus carbonum
  MISC_FEATURE (1)..(221): xylanase
MVSFTSIITAAVAATGALAAPATDVSLVARQNTPNGEGTHNGCFWSWWSDGGARATYTNGAGGSYSVSWGSGGNLVGGKGWNPGTARTITYSGTYNYNGNSYLAVYGWTRNPLVEYYVVENFGTYDPSSQSQNKGTVTSDGSSYKIAQSTRTNQPSIDGTRTFQQYWSVRQNKRSSGSVNMKTHFDAWASKGMNLGQHYYQIVATEGYFSTGNAQITVNCP

SEQ ID NO: 51 - protein, 231 aa, Cochliobolus carbonum
  MISC_FEATURE (1)..(231): xylanase
MVSFKSLLLAAVATTSVLAAPFDFLRERDDVNATALLEKRQSTPSAEGYHNGYFYSWWTDGGGSAQYTMGEGSRYSVTWRNTGNFVGGKGWNPGSGRVINYGGAFNPQGNGYLAVYGWTRNPLVEYYVIESYGTYNPSSGAQIKGSFQTDGGTYNVAVSTRYNQPSIDGTRTFQQYWSVRTQKRVGGSVNMQNHFNAWSRYGLNLGQHYYQIVATEGYQSSGSSDIYVQTQ

SEQ ID NO: 52 - protein, 222 aa, Cochliobolus carbonum
  MISC_FEATURE (1)..(222): xylanase
MVAFTSVLLGLSAIGSAFAAPVADVPDFEFSGPKHLAARQDYNQNYKTGGNIQYNPTSNGYSVTFSGAQDFVLGKGWKQGTTRTVKYTGSTQAQAGTVLVALYGWTRGSKLVEYYIQDFTSGGSGSAQGQKMGQVTCDGSVYDIWQHTQVNQPSIVGTTTFVQYISNRVSKRSTGGTITTKCHFDAWAKLGMNLGNQWDYQTISTEGWGNAAGKSQYTVSAA

SEQ ID NO: 53 - protein, 231 aa, Cochliobolus sativus
  MISC_FEATURE (1)..(231): xylanase
MVSFKSLLLAAVATTSVLAAPFDFLRERDDGNATALLEKRQSTPSSEGYHNGYFYSWWTDGGGSAQYTMGEGSRYSVTWRNTGNFVGGKGWNPGTGRVINYGGAFNPQGNGYLAVYGWTRNPLVEYYVIESYGTYNPSSGAQVKGSFQTDGGTYNVAVSTRYNQPSIDGTRTFQQYWSVRQQKRVGGSVNMQNHFNAWSRYGLNLGQHYYQIVATEGYQSSGSSDIYVQTQ

SEQ ID NO: 54 - protein, 209 aa, Cryptococcus sp. S-2
  MISC_FEATURE (1)..(209): xylanase
MVASAAPVTEAEDGQAATPIAIEKRTGNYVQNYNGNVANFEYSQYDGTFSVNWNGNTDFVCGLGWTVGTGRTITYSGSYNPGYSGSYQAIYGWTGQGSLSEYYVIDNYGGYNPCTGSGVTQLGSLYSDGSSYQVCTHTQYNQPSIVGTTTFPQYFSVRQNKRSSGSVNMQNHFNYWAQHGFPNRNFNYQVLAVEGFSGSGNANMKLISG

SEQ ID NO: 55 - protein, 360 aa, Dictyoglomus thermophilum Rt46B.1
  MISC_FEATURE (1)..(360): xylanase
MFLKKLSKLLLVVLLVAVYTQVNAQTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGSSSGSSGGSSGSTTTTRIECENMSLSGPYVSRITNPFNGIALYANGDTARATVNFPASRNYNFRLRGCGNNNNLARVDLRIDGRTVGTFYYQGTYPWEAPIDNVYVSAGSHTVEITVTADNGTWDVYADYLVIQ

SEQ ID NO: 56 - protein, 225 aa, Emericella nidulans
  MISC_FEATURE (1)..(225): xylanase
MVSFKSLLVLCCAALGAFATPVGSEDLAAREASLLERSTPSSTGWSNGYYYSFWTDGGGDVTYTNGAGGSYTVQWSNVGNFVGGKGWNPGSTRTINYGGSFNPSGNGYLAVYGWTQNPLIEYYIVESYGTYNPGSGGQHRGTVYSDGATYDIYTATRYNAPSIEGTATFEQFWSVRQSKRTGGTVTTANHFNAWAALGMRLGTHNYQIVATEGYQSSGSASITVY

SEQ ID NO: 57 - protein, 608 aa, Fibrobacter succinogenes
  MISC_FEATURE (1)..(608): xylanase
MKTFSVTKSSVVFAMALGMASTAFAQDFCSNAQHSGQKVTITSNQTGKIGDIGYELWDENGHGGSATFYSDGSMDCNITGAKDYLCRAGLSLGSNKTYKELGGDMIAEFKLVKSGAQNVGYSYIGIYGWMEGVSGTPSQLVEYYVIDNTLANDMPGSWIGNERKGTITVDGGTYIVYRNTRTGPAIKNSGNVTFYQYFSVRTSPRDCGTINISEHMRQWEKMGLTMGKLYEAKVLGEAGNVNGEVRGGHMDFPHAKVYVKNGSDPVSSSSVKSSSSTDAPKSSSSKGNGNVSGKIDACKDVMGHEGKETRTQGQNNSSVTGNVGSSPYHYEIWYQGGNNSMTFYDNGTYKASWNGTNDFLARVGFKYDEKHTYEELGPIDAYYKWSKQGSAGGYNYIGIYGWTVDPLVEYYIVDDWFNKPGANLLGQRKGEFTVDGDTYEIWQNTRVQQPSIKGTQTFPQYFSVRKSARSCGHIDITAHMKKWEELGMKMGKMYEAKVLVEAGGGSGSFDVTYFKMTDKAHPLAQPEPESSSSEAKVESSSSTVALHAAPKMELKSGNFQVFDMQGRFLGTVKLDAGASVAQVLKANFKNAGIYMVKQGNFMQRVAVK

SEQ ID NO: 58 - protein, 231 aa, Fusarium oxysporum f. sp. Lycopersici
  MISC_FEATURE (1)..(231): xylanase
MVSFKSLLLAASALTGALAHPFDFLDEQDDGNSTSVLEARQVTGNSEGYHNGYFYSWWSDGGGYANYRMGEGSHYQVDWRNTGNFVGGKGWNPGTGRTINYGGSFSPQGNGYLCVYGWTRSPLVEYYVIESYGTYNPGSAGQHKGTVYNDGDTYDLYQTTRVQQPSIDGTQTFNQYWAIRRNKRTSGAVNMQTIFNAWNSAGMRLGNHYYQILATEGYQSSGSSSIYVQTK

SEQ ID NO: 59 - protein, 295 aa, Fusarium oxysporum f. sp. Lycopersici
  MISC_FEATURE (1)..(295): xylanase
MVHFTSVFAGLSLVAGSLAAPSKEGLFSKITKRAGTPNSSGTNNGFYYSWWSDGGADATYTNGEGGSYSMEWKDGGNVVGGKGWSPGKARTISYEGEYKPNGNSYLSVYGWTRNPLVEYYIVESFGTYNPSSGATKKGTVEADGSTYDIFETTRTNAPSIDGTQTFQQYWSVRQQHRSTGSVDTGLHFDAWEKAGMKLGTHDYQILATEGYFSSGSSHMTVSEGASSGGGAGGSTGGDASQGGDSQQGGDVQQGGDASQGGNGQQGGNGNSFQQPGSENQPQQQEIDTGANEPCQ

SEQ ID NO: 60 - protein, 210 aa, Geobacillus stearothermophilus No. 236
  MISC_FEATURE (1)..(210): xylanase
MKLKKKMLTLLLTASMSFGLFGATSSAATDYWQYWTDGGGMVNAVNGPGGNYSVTWQNTGNFVVGKGWTVGSPNRVINYNAGIWEPSGNGYLTLYGWTRNALIEYYVVDSWGTYRPTGNYKGTVNSDGGTYDIYTTMRYNAPSIDGTQTFQQFWSVRQSKRPTGSNVSITFSNHVNAWRSKGMNLGSSWAYQVLATEGYQSSGRSNVTVW

SEQ ID NO: 61 - protein, 231 aa, Gibberella zeae 180378
  MISC_FEATURE (1)..(231): xylanase
MVSFKSLLVAVSALTGALARPFDFLDERDDGNATSVLEARQVTGNSEGYHNGYFYSWWSDGGGYAQYRMGEGSHYQVDWRNTGNFVGGKGWNPGTGRTINYGGSFNPQGNGYLCVYGWTRGPLVEYYVIESYGSYNPGSQAQHRGTVYTDGDTYDLYMSTRYQQPSIDGVQTFNQYWSIRRNKRTSGSVNMQNHFNAWRSAGMNLGNHYYQILATEGYQSSGSSSIYVQTS

SEQ ID NO: 62 - protein, 227 aa, Helminthosporium turcicum
  MISC_FEATURE (1)..(227): xylanase
MVSFTSIITAAVAATGALAAPATDIAARAPSDLVARQSTPNGEGTHNGCFYSWWSDGGARATYTNGAGGSYSVSWGTGGNLVGGKGWNPGTARTITYSGQYNPNGNSYLAIYGWTRNPLVEYYVVENFGTYDPSSQAQNKGTVTSDGSSYKIAQSTRTNQPSIDGTRTFQQYWSVRQNKRSSGSVNMKTHFDAWASKGMNLGSHYYQIVATEGYFSSGSASITVNCP

SEQ ID NO: 63 - protein, 227 aa, Humicola grisea var. thermoidea 60849
  MISC_FEATURE (1)..(227): xylanase
MVSIKSVLAAATAVSSALAAPFDFVPRDNSTALQARQVTPNAEGWHNGYFYSWWSDGGGQVQYTNLEGSRYQVRWRNTGNFVGGKGWNPGTGRTINYGGYFNPQGNGYLAVYGWTRNPLVEYYVIESYGTYNPGSQAQYKGTFYTDGDQYDIFVSTRYNQPSIDGTRTFQQYWSIRKNKRVGGSVNMQNHFNAWQQHGMPLGQHYYQIVATEGYQSSGESDIYVQTH

SEQ ID NO: 64 - protein, 227 aa, Humicola insolens
  MISC_FEATURE (1)..(227): xylanase
MVSLKSVLAAATAVSSAIAAPFDFVPRDNSTALQARQVTPNAEGWHNGYFYSWWSDGGGQVQYTNLEGSRYQVRWRNTGNFVGGKGWNPGTGRTINYGGYFNPQGNGYLAVYGWTRNPLVEYYVIESYGTYNPGSQAQYKGTFYTDGDQYDIFVSTRYNQPSIDGTRTFQQYWSIRKNKRVGGSVNMQNHFNAWQQHGMPLGQHYYQVVATEGYQSSGESDIYVQTH

SEQ ID NO: 65 - protein, 229 aa, Hypocrea jecorina
  MISC_FEATURE (1)..(229): xylanase
MVAFSSLICALTSIASTLAMPTGLEPESSVNVTERGMYDFVLGAHNDHRRRASINYDQNYQTGGQVSYSPSNTGFSVNWNTQDDFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 66 - protein, 222 aa, Hypocrea jecorina
  MISC_FEATURE (1)..(222): xylanase
MVSFTSLLAASPPSRASCRPAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 67 - protein, 190 aa, Hypocrea lixii E58
  MISC_FEATURE (1)..(190): xylanase
QTIGPGTGYSNGYYYSYWNDGHAGVTYTNGGGGSFTVNWSNSGNFVAGKGWQPGTKNKVINFSGSYNPNGNSYLSIYGWSRNPLIEYYIVENFGTYNPSTGATKLGEVTSDGSVYDIYRTQRVNQPSIIGTATFYQYWSVRRNHRSSGSVNTANHFNAWASHGLTLGTMDYQIVAVEGYFSSGSASITVS

SEQ ID NO: 68 - protein, 283 aa, Lentinula edodes Stamets CS-2
  MISC_FEATURE (1)..(283): xylanase
MAYKSLLFLALIAVTATALDVFDNSTEVIGKRSIPNGEGTNNGYFYSVYSDTTVTGTYTNGPGGEYTLTWGGSGDVVVGKGWNPGGPMSVEYSGTYSPNGNSYLSVYGWMTSPLVEYYITDSFGDYNPSTGGTHLGTCTSDGGVYDIYTQTRTNAPSIQGTATFQQYWSIRQTHRVGGTVTTGNHYSCWESVGLPLGTFNYIILATEGYSSSGTSTITVGQGTGTGSSAPSGPSSTTTTPPTAPTGGTVAQWGQCGGIGYSGPTTCASPYTCTVANAYYSQCL

SEQ ID NO: 69 - protein, 233 aa, Magnaporthe grisea
  MISC_FEATURE (1)..(233): xylanase
MVSFTSIVTAVVALAGSALAIPAPDGNMTGFPFEQLMRRQSTPSSTGRHNGYYYSWWTDGASPVQYQNGNGGSYSVQWQSGGNFVGGKGWMPGGSKSITYSGTFNPVNNGNAYLCIYGWTQNPLVEYYILENYGEYNPGNSAQSRGTLQAAGGTYTLHESTRVNQPSIEGTRTFQQYWAIRQQKRNSGTVNTGEFFQAWERAGMRMGNHNYMIVATEGYRSAGNSNINVQTPA

SEQ ID NO: 70 - protein, 266 aa, Neocallimastix frontalis
  MISC_FEATURE (1)..(266): xylanase
MKMVLGVLMVKNGVVVVPAVHPLVVVTMLHQVVVDTKVLMELTSVTTLSTLAESVKVTSNKVGDINGIGYELWADSGNNSATFYDDGSFSCSFQRAKDYLCRSGLSFDSTKTHKQIGHIYAEFKLVKQNIQNVDYSYVGIYGWTRNPLVEFYVVDNWLSQWRPGDWVGNKKHGDFTIDGAQYTVYENTRYGPSIDGDTNFKQYFSIRQQPRDCGTIDITAHFEQWEKLGMTMGKMHEAKVLGEAGSNNGGTSGTADFPFAKVYVKN

SEQ ID NO: 71 - protein, 337 aa, Neocallimastix patriciarum
  MISC_FEATURE (1)..(337): xylanase
TVAKAQWGGGASAGQKLSVGGGQNQHKGVSDGFSYEIWLDNTGGSGSMTLGSGATFKAEWNAAVNRGNFLARRGLDFGSQKKATDYSYIGLDYTATYRQTASASGNSRLCVYGWFQNRGVQGVPLVEYYIIEDWVDWVPDAQGKMVTIDGAQYKIFQMDHTGPTINGGSETFKQYFSVRQQKRTSGHITVSDHFKEWAKQGWGIGNLYEVALNAEGWQSSGVADVTLLDVYTTPKGSSPATSAAPRTTTRTTTRTKSLPTNYNKCSARITAQGYKCCSDPNCVVYYTDDDGTWGVENNEWRGCGVEQCSSKITSQGYKCCSDPNCVVFYTDDDGKWG

SEQ ID NO: 72 - protein, 607 aa, Neocallimastix patriciarum
  MISC_FEATURE (1)..(607): xylanase
MRTIKFFFAVAIATVAKAQWGGGGASAGQRLTVGNGQTQHKGVADGYSYEIWLDNTGGSGSMTLGSGATFKAEWNASVNRGNFLARRGLDFGSQKKATDYSYIGLDYTATYRQTGSASGNSRLCVYGWFQNRGVQGVPLVEYYIIEDWVDWVSDAQGRMVTIDGAQYKIFQMDHTGPTINGGSETFKQYFSVRQQKRTSGHITVSDHFKEWAKQGWGIGNLYEVALNAEGWQSSGIADVTKLDVYTTQKGSNPAPTSTGTVPSSSAGGSTANGKKFTVGNGQNQHKGVNDGFSYEIWLDNTGGNGSMTLGSGATFKAEWNAAVNRGNFLARRGLDFGSQKKATDYDYIGLDYAATYKQTASASGNSRLCVYGWFQNRGLNGVPLVEYYIIEDWVDWVPDAQGKMVTIDGAQYKIFQMDHTGPTINGGSETFKQYFSVRQQKRTSGHITVSDHFKEWAKQGWGIGNLYEVALNAEGWQSSGVADVTLLDVYTTPKGSSPATSAAPRTTTRTTTRTKSLPTNYNKCSARITAQGYKCCSDPNCVVYYTDEDGTWGVENNDWCGCGVEQCSSKITSQGYKCCSDPNCVVFYTDDDGKWGVENNDWCGCGF

SEQ ID NO: 73 - protein, 607 aa, Neocallimastix patriciarum MCH3
  MISC_FEATURE (1)..(607): xylanase
MRTIKFFFAVAIATVAKAQWGGGGASAGQRLTVGNGQTQHKGVADGYSYEIWLDNTGGSGSMTLGSGATFKAEWNASVNRGNLLARRGLDFGSQKKGKPITATLIGYTATYRQTGSASGNSRLCVYGWFQNRGVQGVPLVEYYIIEDWVDWVPDAQGRMVTIDGAQYKIFQMDHTGPTINGGSETFKQYFSVRQQKRTSGHITVSDHFKEWAKQGWGIGNLYEVALNAEGWQSSGIADVTKLDVYTTQKGSNPAPTSTGTVPSSSAGGSTANGKKFTVGNGQNQHKGVNDGFSYEIWLDNTGGNGSMTLGSGATFKAEWNAAVNRGNFLARRGLDFGSQKKANRFDYIGLDYAATYKQTASASGNSRLCVYGWFQNRGLNGVPLVEYYIIEDWVDWVPDAQGKMVTIDGAQYKIFQMDHTGPTINGGSETFKQYFSVRQQKRTSGHITVSDHFKEWAKQGWGIGNLYEVALNAEGWQSSGVADVTLLDVYTTPKGSSPATSAAPRTTTRTTTRTKSLPTNYNKCSARITAQGYKCCSDPNCVVYYTDEDGTWGVENNDWCGCGVEQCSSKITSQGYKCCSDPNCVVFYTDDDGKWGVENNDWCGCGF

SEQ ID NO: 74 - protein, 220 aa, Neurospora crassa OR74A
  MISC_FEATURE (1)..(220): xylanase
MVSLKSLLLGAAGALAMPFNATEFSELAERGGTPSSTGFNNGFYYSFWTDNGGNVNYANGASGSYSVNWQNAGNFVAGKGWNPGSARTITYSGNFRPSGNGYLSVYGWTRNPLVEYYVVENFGSYNPSSGAQRLGSVYTDGSTYDIYKTTRYNQPSIDGTRTFNQYWSVRQQKRTGGTVTMANHFNAWAKAGLHLGTHNYQIVATEGYQSSGSAQITVHG

SEQ ID NO: 75 - protein, 293 aa, Neurospora crassa OR74A
  MISC_FEATURE (1)..(293): xylanase
MVAFSSILLAASTAISGVFALPGELPGQGSLFKRQTYTSSATGTHNGYYFSFWTDGQGSVRYTNEAGGQYTATWSGNGNWVGGKGWMPGTDRTINYTGTYSPNGNSYLAVYGWTRNPLIEYYVVENFGTYNPSTGATRLGSVTSDGGVYDIYRTQRVNQPSIDGTATFYQYWSVRQQKRTGGSVNMKNHFDAWTRSGLTLGTHNYQIVATEGYFSSGSSTINVGASGGSGGGGGGTTPENPGTTTTPTNPGNGGGGSCAAKWGQCGGQGWSGATCCQSGSTCQAANQWYSQCV

SEQ ID NO: 76 - protein, 344 aa, Nonomuraea flexuaosa
  MISC_FEATURE (1)..(344): xylanase
MNEPLTITQARRRRRLGLRRIVTSAFALALAIAGALLPGTAHADTTITQNQTGYDNGYFYSFWTDAPGTVSMTLHSGGSYSTSWRNTGNFVAGKGWSTGGRRTVTYNASFNPSGNAYLTLYGWTRNPLVEYYIVESWGTYRPTGTYKGTVTTDGGTYDIYETWRYNAPSIEGTRTFQQFWSVRQQKRTSGTITIGNHFDAWARAGMNLGSHDYQIMATEGYQSSGSSTVSISEGGNPGNPGNPGNPGNPGNPGNPGGGCVATLSAGQQWSDRYNLNVSVSGSNNWTVRMDVPYPARIIATWNIHAQWPESQVLIARPNGNGNNWGVTIQHNGNWTWPTVTCTAN

SEQ ID NO: 77 - protein, 362 aa, Orpinomyces sp. PC-2
  MISC_FEATURE (1)..(362): xylanase
MRTIKFLFALAITTVAKAQWGGNGGASAGQRLSVGGGQNQHKGVFDGFSYEIWLDNTGGSGSMTLGKGATFKAEWSAAVNRGNFLARRGLDFGSTKKATAYEYIGLDYEASYRQTASASGNSRLCVYGWFQNRGVQGVPLVEYYIIEDWVDWVPDAQGKMVTIDGAQYKIFQMDHTGPTINGGNETFKQYFSVRQQKRTSGHITVSDHFKAWSNQGWGIGNLYEVALNAEGWQSSGVADVPKLDVYTTKQGSAPRTTTTTTRTTTRTTTKTLPTTNKKCSAKITAQGYKCCSDPNCVVYYTDEDGTWGVENNQWCGCGVEACSGKITAQGYKCCSDPKCVVYYTDDDGKWGVENNEWCGCGL

SEQ ID NO: 78 - protein, 194 aa, Paecilomyces varioti Brainier
  MISC_FEATURE (1)..(194): xylanase
GTTPNSEGWHDGYYYSWWSDGGGDSTYTNNSGGTYEITWGNGGNLVGGKGWNPGLNARAIHFTGVYQPNGTSYLSVYGWTRNPLVEYYIVENFGSSNPSSGSTDLGTVSCDGSTYTLGQSTRYNAPSIDGTQTFNQYWSVRQDKRSSGTVQTGCHFDAWASAGLNVTGDHYYQIVATEGYFSSGYARITVADVG

SEQ ID NO: 79 - protein, 282 aa, Penicillium funiculosum
  MISC_FEATURE (1)..(282): xylanase
MGISSILLSALIAGGALALPAAEPVSFDIRDENITLARRAEAINYNQDYIASGANVQYSPNMAAGSFSINYNTQGDFVVGLGWQPGDANPITYSGSFSASGVGILAVYGWSTNPLVEYYVMEVHDGYQTAGTHKGTVTTDGGTYDIWEHQQVNQPSILGTSTFNQYISIRQSPRTSGTVTVQNHFNAWAQAGMNLGTLNYQVMAVESWSGSGSGQISLSKGTGGGSTTTTPTGPTSTSTAPSSGGTGAAQWGQCGGIGWTGPTTCVAPYTCKYENAYYSQCQ

SEQ ID NO: 80 - protein, 223 aa, Penicillium funiculosum
  MISC_FEATURE (1)..(223): xylanase
MKLFLAAIVLCATAATAFPSELAQRAAGDLSKRQSITTSQTGTNNGYYYSFWTNGGGEVTYTNGDNGEYSVTWVDCGDFTSGKGWNPANAQTVTYSGEFNPSGNAYLAVYGWTTDPLVEYYILESYGTYNPSSGLTSLGQVTSDGGTYDIYSTQRVNQPSIEGTSTFNQYWSVRTEKRVGGTVTTANHFAAWKALGLEMGTYNYMIVSTEGYESSGSSTITVS

SEQ ID NO: 81 - protein, 208 aa, Penicillium purpurogenum
  MISC_FEATURE (1)..(208): xylanase
MKVTAAFAGLLARHSPPLSTELVTRSINYVQNYNGNLGAFSYNEGAGTFSMYWQQGVSNDFVVGLGRSTGSSNPITYSASYSASGGSYLAVYGWVNSPQAEYYVVEAYGNYNPCSSGSATNLGTVSSDGGTYQVCTDTRVNQPSITGTSTFTQFFSVRQGSRTSGTVTIANHFNFWANDGFGNSNFNYQVVAVEAWSGTGTASVTVSA

SEQ ID NO: 82 - protein, 217 aa, Phaedon cochleariae
  MISC_FEATURE (1)..(217): xylanase
MQFLIPVVILCVSLVDSQKVLYNNEIGFNNGFYYAFWKDSGSATFTLESGGRYAGNWTTSTNNWVGGKGWNPGNSWRTVNYSGYYGINEYANSYLSLYGWTTNPLIEYYVVESYGSYSPLNCPGGTDEGSFTSGGATYQVRKCRRTNAPSIIGTQSFDQYFSVRTPKKGFGQVSGSVNFADHVQYWASKGLPLGTHAHQIFATEGYQSSGFADITVS

SEQ ID NO: 83 - protein, 290 aa, Phanerochaete chrysosporium ME  446
  MISC_FEATURE (1)..(290): xylanase
MVSFNSLLVAVSAATCALAFPFEFHNGTHVFPRQSTPAGTGTNNGYFYSFWTDGGGSVTYNNGPAGEYSVTWSNADNFVAGKGWNPGSAQAISFTANYQPNGNSYLSVYGWSTNPLVEYYILEDFGTYNPAVSLTHKGTLTSDGATYDVYEGTRVNEPSIQGTATFNQYWSIRSSKRSSGTVTTANHFAAWKQLGLPLGTFNYQIVATEGYQSSGSSTVTVNPAGGVTSPTAPTGPSSVSTTPSGPSSSPSPVGSCAALYGQCGGQGWTGPTCCSSGTCKFSNNWYSQCL

SEQ ID NO: 84 - protein, 381 aa, Pichia stipitis
  MISC_FEATURE (1)..(381): xylanase
MTVYKRKSRVLIAVVTLLHVLSHAPTKMLTTDVLLTRCMHLCHFRTSDSVYTNETSEERSMSDRLNITRVMSYDRWTDLVGELEVRELKHVMSHRTYSLCDLSCSTVLDSNSMFSLGKGWQAISSRQGVGATVYGWTRSPLLIEYYVVDSWGSYHPSNTITGTFVTVKCDGGTYDIYTAVRVNAPSIEGTTFTQYWSVRQSATIQLAVIKPLTLQNATITFTFSNHFDAWKTMTLEATHSTEGYFSSGITYEQPHQPHRNTWATSLTSQTKHTARSLPINLDRDKQCSQNESQLAASIYCRATLSTPKYWTTKGQNTNFKRASQGTNSDISLRRNNCIQAKIERHKQVRTHTKHRQIAQNRGRAWHTVRQTQKYRGQKWEK

SEQ ID NO: 85 - protein, 625 aa, Piromyces sp.
  MISC_FEATURE (1)..(625): xylanase
MKLFQIFPLLLSLTSVTLAADDFCNATGFQGQSVVSTGHDVKKIGNIDYEQWADGGNNSATFYSDGSFKCNFSNTKDYLCRSGVAFSQAKYPSEIGHIEAEYRLVKKSASNVGYSYVGVYGWTLQSGISGVYEYYIVDNWLSQWRPGDWVGNTKFGDFTIDGGVYTVYKNVNGNLTQYFSLRKSERTCGTIDVTAHFAQWEKLGLKMPKITEIKVLAEAGNTGGGCSGSVEIPYAKIYINGKDQDGKSKGGSSSGGSNGQGLGNGQGNGQGQGNGQGQSATGSGKCPSTITSQGYKCCSSNCDIIYRDQSGDWGVENDEWCGCGSRVPKTTNCPSSIKNQGYKCCSDSCEIVLTDSDGDWGIENDEWCGCGIKNTTPTTTTKKSNNSQPTQGQSNNNSSTNTNFCSTSKHSGQSVTETSNKVGSIGGVGYELWADSGNNSATFYSDGSFSCSFRNAKDYLCRSGLSFDSTKTYQQLGHMYADFKLVKQNIQNVDYSYVGIYGWTRNPLVEFYVVDNWLSQWRPGDWVGNKKHGDFTIDGAKYTVYENTRTGPSIDGNTTFKQYFSIRQQARDCGTIDITAHFEQWEKLGMRMGKMHEAKVLGEAGSTGSGTSGTADFPYAKVYIK

SEQ ID NO: 86 - protein, 235 aa, Polyplastron multivesiculatum
  MISC_FEATURE (1)..(235): xylanase
EKVICLLIALFGLIEAQTFYNNAQGQIDGLDYELWKDTGTTSMTLLGGGKFSCSWSNINNCLFRIGKKWNCQYEWWELGTVLVNYDVDYNPNGNSYLCIYGWTRNPLVEYYIVESWGSWRPPGGSPMNTMYVDDGQYDVYVTDRINQPSIDGNTNFKQYWSVRTQKKTRGTVHVNHHFYNWQEMGLKVGKVYEASLNIEGYQSAGSATVNKNEVVQTTEQIGLIISSNLDEILYL

SEQ ID NO: 87 - protein, 346 aa, Pseudomonas sp. ND137
  MISC_FEATURE (1)..(346): xylanase
MHAKKSLAILTTAAAVLSVAATQAQTLTSNQTGTHNGYYYSFWTDAPGTVSMTLGSGGNYSSQWSNTGNWVGGKGWNPGGRRTVSYSGTFNPSGNGYLTLYGWTTSPLIEYYIVDNWGSYRPGESGTYYGTVNTDGGTYDIYRTQRVNQPSIQGTATFYQYWSVRQQKRTGGTITTGNHFDAWASHGLNLGNHDYMVMATEGYQSSGNSNITLGTTGGGNNGGGSNNGGDVTVRARGVSGSEHINLRVGGATVADWTLTTSNQSYTYSGGASGDVNVEYDNDASGRDVILDYVTVNGENRQAEDMEYNTATYANGECGGGSYSETMHCSGVIGFGHTDDCFSGNCN

SEQ ID NO: 88 - protein, 680 aa, Ruminococcus albus
  MISC_FEATURE (1)..(680): xylanase
MRNNFKMRVMAGVAAVICLAGVLGSCGNSSDKDSSSKKSADSAKADSNKDSKNGQVFTKNARGTSDGYDYELWKDKGDTEMTINEGGTFSCKWSNINNALFRRGKKFDCTKTYKELGNISVKYGVDYQPDGNSYMCVYGWTIDPLVEFYIVESWGSWRPPGAAESLGTVTVDGGTYDIYKTTRYEQPSIDGTKTFDQYWSVRQDKPTGDGTKIEGTISISKHFDAWEQVGLTLGNMYEVALNIEGYQSNGQATIYENELTVDGNYSADPAPEVKKSSDEKEPEPQSTNGAGLFTADFESDECGWTSRGSAAVAQNTDEASEGRGSLFVSGRTDNWNGASIELDSGTFKQGSTYSFKTDAMQKSGKEATLKFTMQYTAADGDHYDQIAQQTAPDGEWITLEGDYTIPDDATKLVLYVESPDSLTDFYIDNASATGDGSEPAKTADDKKEEAEYTFSDPVAVSNTADISWIDKDKPMVAIAFDDGASAAKKGDPAYRIIDTMADNGFHATFFYIGSQIKTEEQVKYAHEKGMETANHTMTHPYLSKLKPAEIRDEYEKCREKLKGIIGEEPSALMRLPYLDGGGDTTRTLNDVALISCSVDTKDWNKATSDKIVDTIKKAKDNGSLNGAIVLCHENYAATAEAMEEIVPWLKDEGWQVVTVSELFAANGKELMGGKIYSKLS

SEQ ID NO: 89 - protein, 828 aa, Ruminococcus albus
  MISC_FEATURE (1)..(828): xylanase
MKKPVSKRIASAFVGGVLMVSPIISSVTASAAKTLTTSPSHTQEVGWYNDYHHEIWQADTPNSSTMTLHDNDGGFSTQWKCGPNNSRGNFLARRGLYWGLNNPKTYKDYKPLYCDYDCSWYAGSSGNSRICIYGWAQNPLVEYYIIEDWKNWSPAQDGSAQYKGQTTIDGSVFKVYTTSRNSYTIEANKNFTQYISIRQNLRTKGTISVSEHFKAWESMGMKMGNLYEVAFNVEGWESDGQADVTLNMYEGESGYHPDPEPEESEIEGTLYSATFENGTNYWSGRGSASVASSSSKAYEGSKSLYVSGRTDNWNGGEVEVNTSTFKPGNSYSFSAMVNPAESTTVQLSMQYDQNGTTKYTQIAEGSCTAGKWTKLENTSFTIPSGASNVKMYVEAPDSLCDIYVDRAIIADKGYVASGDTPVYNNANVPSNVKMEYNSQYKQMKFTWDAVNGADKYGIAVYLAGKWRVQDSNISGTSYTTPKGLTPGMSYKVAIAARVNGKWDTTAAIDNYVVVTTRSYNNIQYHTPSNTNTNTNTNTNTNTNNNQQPETSSYNYASNMQFKEAPGYYFNSCSQAGKVVKESYNGINGYNSLNVYLPYGYDSSKKYNIFYLMHGGGENENTLFYQDDTMMQNLFDNMIKNGELDPLIVVTPTFNKTEAGKFYNEFRQSVVPFVEGKYSTYAGRNTSQSSLQASRMHRAYGGFSMGGVSTWAVMENCLDIVGYFMPLSGDHWNGNSGYDKAKSIANAIDRSGLQKNQYFIFAATGSDDIAYPNVNPQINEMKKMSQFVYTSDFSKGNLYFLVASGKTHWWGYVRHYVYDALPSFFHEGQ

SEQ ID NO: 90 - protein, 792 aa, Ruminococcus flavefaciens 17
  MISC_FEATURE (1)..(792): xylanase
MKTKKIISGLLSAFIIASSMPAALSAYADDQQEKGNVGGYDWEMWNQNYTGTVSMKPSAGSFTCSWSGIENFLARMGKNYDSQKINYKALGDIVLSYDVEYTPRGNSYMCIYGWTRNPLMEYYIVEGWGDWEPPGNDGVDNFGTATIDGRTYKIRKSMRYNQPSIEGTKTFPQYWSVRTSSGSRNNTTNYMKDSVTVSAHFDAWSKAGLDMSGTLYEVSLNIEGYRSNGSANVKSITVGGDPPPPKEPVKADENGYYLKENFESGTGDWGARGSAKVESSSTGYDGSKGIYVSGRTNNWNGASIKLDTSTFVPGETYSLGAAVMQDSEASVDLKLTLQYTDANGKDNYDEVKTVSAAKGQWTDLSNGSYTIPADASGLELYIEAPDSLTDFYVDGAYAGVKGTKPLLSISNSTNPTDPTDPTNPTINTGIRGDVNEDGVIDSLDMIALRRGIIKIMSGGGIAPPNSDINGDGDVSVADLVALKRFILGIDKKFADPVTTTTVTTTKITTTTTTSTPATGKNLNEQIRKDMPTSVPSGAEQSSQCKVEKKTYMCKFTGKQKSCNVILPPNYDPSKKYPVMYILHGIMGSENDMMQGFGTQGLMTGLMKSGQAEEFIAVTPNMFTSKTMSGPNGINQQTCEQYDNFLYDISESLIPFIEENYPVKTGRENRAITGFSMGGREAIYIGLMRPDLFAYVGGACPAPGITPGSDTFMSHPGCMKESEMKFRDVGPEPNVFMITGGTNDGTVGTFPKQYSDILTRNGVDHVYQSIPGGQHGDNSVKPHLYTFFRYAFK

SEQ ID NO: 91 - protein, 954 aa, Ruminococcus flavefaciens 17
  MISC_FEATURE (1)..(954): xylanase
MKLSKIKKVLSGTVSALMIASAAPVVASAADQQTRGNVGGYDYEMWNQNGQGQASMNPGAGSFTCSWSNIENFLARMGKNYDSQKKNYKAFGNIVLTYDVEYTPRGNSYMCVYGWTRNPLMEYYIVEGWGDWRPPGNDGEVKGTVSANGNTYDIRKTMRYNQPSLDGTATFPQYWSVRQTSGSANNQTNYMKGTIDVTKHFDAWSAAGLDMSGTLYEVSLNIEGYRSNGSANVKSVSVTQGGSSDNGGQQQNNDWNQQNNNQQQNNDWNNWGQQNNDWNQWNNQGQQNNDWNNWGQQNNDWNQWNNQGQQQNNDWNNWGQQNNDWNQWNNQGQQQNNDWNNWGQQNNDWNQWNNQGQQQNNDWNNWGQQNNDWNQWNNQNNNQQNAWNGWDNNNNWNQNNQQQNNWDWNNQNNWNNNQQQNNDWNQWNNQNNWNNNQQQNNDWNQWNNQGQQNNDWNQWNNQNNWNQNNNQQNAWNGWDNNNNWNQWDQNNQWNNQQQNNTWDWNNQNNWNNNQQNNDWNQWNNQGQQQNNDWNQWNNQNNNQNNGWDWNNQNNWNQNNNQQNAWNGWDNNNNWNQWGGQNNDWNNQQQNNDWNQWNNQGQQQNNDWNNQNNWNQGQQNNNNSAGSSDSLKGAFSKYFKIGTSVSPHELNSGADFLKKHYNSITPENELKPESILDQGACQQKGNNVNTQISLSRAAQTLKFCEQNGIALRGHTFVWYSQTPDWFFRENFSQNGAYVSKDIMNQRLESMIKNTFAALKSQYPNLDVYSYDVCNELFLNNGGGMRGADNSNWVKIYGDDSFVINAFKYARQYAPAGCKLYLNDYNEYIPAKTNDIYNMAMKLKQLGYIDGIGMQSHLATNYPDANTYETALKKFLSTGLEVQITELDITCTNSAEQADLYEKIFKLAMQNSAQIPAVTIWGTQDTVSWRSSQNPLLFSAGYQPKPAYDRVMALAK

SEQ ID NO: 92 - protein, 781 aa, Ruminococcus flavefaciens 17
  MISC_FEATURE (1)..(781): xylanase
MRFKALKKILSGTVSALMIAVSMPTSFSVHADDQQDKGNVGGYDWEMWNQNYTGTVSMKPSAGSFTCSWSGIENFLARMGKNYDSQKINYKALGDIVLSYDVEYTPRGNSYMCIYGWTRNPLMEYYIVEGWGDWEPPGNDGVDNFGTATIDGRTYKIRKSMRYNQPSIEGTKTFPQYWSVRTSSGSRNNTTNYMKDSVTVSAHFDAWSKAGLDMSGTLYEVSLNIEGYRSNGSANVKSITVGGDPPPPKEPVKADENGYYLKENFESGTGDWGARGSAKVESSSTGYDGSKGIYVSGRTNNWNGASIKLDTSTFVPGETYSLGAAVMQDSEASVDLKLTLQYTDANGKDNYDEVKTVSAAKGQWTDLSNASYTIPADASGLELYIEAPDSLTDFYVDGAYAGVKGTKPLLSIDPSQQPVITDPITTQPPSSNVDKNKTIKILPAGDSITNGDGEQGGYRKYLFDALSKLGYTKIDMVGPNRDRSNTANGITYDTDHAGFSGYQIKEVPSWGQQQGGKGSLYNELKNNDVVKKTQPDIILLMIGTNDLTANRSMDACTADLRSMLDYMLGDMPSGGMIFLCSVPEHTAYGGNTQKIANYNNTVKSVAEEYANKGKNVRFADVHGCLDGMNDISSQDHLHPNGTGYKKMGNFFAGVIDDYITSLAPPVTTTTSTTTTTTTTTTTTTTTTTATTTTTTTTTAEPELTVKKAGDANDDGNLDLADAILIMQALANPNKYGLEGTAEKHLTEQGKVNADVDKSTKGLTADDALMIQEFLIGKVASL

SEQ ID NO: 93 - protein, 802 aa, Ruminococcus flavefaciens 17
  MISC_FEATURE (1)..(802): xylanase
MKKSIFKRYAAAVGLMASVLMFTAVPTTSNAADDQKTGKVGGFDWEMWNQNYTGTVSMNPGAGSFTCSWSGIENFLARMGKNYDDQKKNYKAFGDIVLTYDVEYTPRGNSYMCIYGWTRNPLMEYYIVEGWGDWEPPGNDGVDNFGTTTIDGKTYKIRKSMRYNQPSIEGTKTFPQYWSVRTTSGSRNNTTNYMKDQVSVTKHFDAWSKAGLDMSGTLYEVSLNIEGYRSNGSANVKSISFDGGIDIPDPEPIKPDENGYYLKENFESGEGNWSGRGSAKVKSSSGYDGTKGIFVSGREDTWNGASINLDELTFKAGETYSLGTAVMQDFESSVDFKLTLQYTDADGKENYDEVKTVTAAKGQWVDLSNSSYTIPSGATGLVLYVEVPESKTDFYMDGAYAGVKGTKPLISISSQSVDPPVTEPTNPTNPTGPSVTKWGDANCDGGVDLSDAIFIMQFLANPNKYGLTGTETNHMTNQGKVNGDVCEHGSGLTEDDAVSIQKYLIRAISELPESYLEGHDPSKTTTTTTRITTTTTTTTTTTTSKTTTTTTTTSPAMHGGYRDLGTPMNTSATMISDFRTGKAGDFFASDGWTNGKPFDCWWYKRNAVINDGCLQLSIDQKWTNDKNPDWDPRYSGGEFRTNNFYHYGYYECSMQAMKNDGVVSSFFTYTGPSDDNPWDEIDIEILGKNTTQVQFNYYTNGQGKHEKLYDLGFDSSEAYHTYGFDWQPNYIAWYVDGREVYRATQDIPKTPGKIMMNAWPGLTVDDWLKAFNGRTPLTAHYQWVTYNKNGVQHSSQGQNPWG

SEQ ID NO: 94 - protein, 789 aa, Ruminococcus sp.
  MISC_FEATURE (1)..(789): xylanase
MKKTVKQFISSAVTALMVAASLPAVPSVNAADAQQRGNIGGFDYEMWNQNGQGQVSMTPKAGSFTCSWSNIENFLARMGKNYDSQKKNYKAFGDITLSYDVEYTPKGNSYMCVYGWTRNPLMEYYIVEGWGDWRPPGNDGENKGTVTLNGNTYDIRKTMRYNQPSLDGTATFPQYWSVRQKSGSQNNTTNYMKGTISVSKHFDAWSKAGLDMSGTLYEVSLNIEGYRSSGNANVKAISFDGSIPEPTSEPVTQPVVKAEPDANGYYFKEKFESGAGDWSARGTGAKVTSSDGFNGSKGILVSGRGDNWHGAQLTLDSSAFTAGETYSFGALVKQDGESSTAMKLTLQYNDASGTANYDKVAEFTAPKGEWVDLSNTSFTIPSGASDLILYVEAPDSLTDFYIDNAFGGIKNTSPLEDVGSHTISTPGSETTTVTTASNKGIRGDINGDGVINSFDLAPLRRGILKMMSGSGSTPENADVNGDGTVNVADLLLLQKFILGMEKSFPDPVTTTTTKPITTTTEKIVTTTTSSSSSSSGKNLNADIRKDMPTSVPGGNEKSGGCKVEKKTYNCKFTGGQKSCNVILPPNYSASKQYPVMYVLHGIGGNEGSMVSGMGVQELLAGLTANGKAEEMIIVLPSQYTSKNGNQGGGFGINQEVCAAYDNFLYDISDSLIPFIEANYPVKTGRENRAITGFSMGGREAIYIGLMRPDLFAYVGGACPAPGITPGKDMFMEHPGCMQESEMKFRDVGPEPNVFMITGGTNDGVVGTFPKQYSDILTRNGVDQRLPVYP

SEQ ID NO: 95 - protein, 197 aa, Schizophyllum commune
  MISC_FEATURE (1)..(197): xylanase
SGTPSSTGTDGGYYYSWWTDGAGDATYQNNGGGSYTLTWSGNNGNLVGGKGWNPGAASRSISYSGTYQPNGNSYLSVYGWTRSSLIEYYIVESYGSYDPSSAASHKGSVTCNGATYDILSTWRYNAPSIDGTQTFEQFWSVRNPKKAPGGSISGTVDVQCHFDAWKGLGMNLGSEHNYQIVATEGYQSSGTATITVT

SEQ ID NO: 96 - protein, 205 aa, Scytalidium acidophilum
  MISC_FEATURE (1)..(205): xylanase
MKFTLTIAGLLAVGSTAAPTTEKRNPGGIDYVQNYNGDVADFQYNEGAGTYTCGWDGSTDFVVGLGWSTGAARDITYSATYNAGGSGSYLAVYGWVNSPQAEYYIVESYGDYNPCSNAEGLGTLESDGSTYTVCTDTRTNEPSITGTSTFTQYWSVRQSERTSGTVTVGNHFNYWAQHGFGDSYNFQVMAVEAFSGSGSASVSVS

SEQ ID NO: 97 - protein, 235 aa, Scytalidium thermophilum Af101-3
  MISC_FEATURE (1)..(235): xylanase
MVSFKTLLLAVVGAAAASPIALPFDPAEVTARHARGENVTEFLLARGGTPSSTGWHGGYFYSFWTDGGGEVNYWNGNNGNYGVNWRNCGNFVGGKGWKPGAARTINYSGTFNPSGNGYLAIYGWTTNPLVEYYIVENFGTYDPSSQAQNLGTFYVDGSNYKIAKSTRYNQPSIIGTATFNQYWSVRQNKRSSGSVNVGAHFQAWAQRGLNLGNHDYQIVATEGYQSSGSASITVW

SEQ ID NO: 98 - protein, 231 aa, Setosphaeria turcica
  MISC_FEATURE (1)..(231): xylanase
MVSFKSLLLAAVATTSVFAAPFDFLAERDDGNATALLEKRQSTPSSEGYHNGYFYSWWTDGGGSAQYTMGEGSKYSVTWRNTGNFVGGKGWNPGNGRTINYGGSFNPQGNGYLAVYGWTRNPLVEYYVIESYGTYNPSSGAQRKGSFQTDGGTYDIATSTRYNQPSIDGTRTFQQYWSVRTQKRVGGSVNMQNHFNAWSRYGMNLGTHYYQIVATEGYQSSGNSEIYVQTM

SEQ ID NO: 99 - protein, 241 aa, Streptomyces coelicolor A3
  MISC_FEATURE (1)..(241): xylanase
MQQDGTQQDRIKQSPAPLNGMSRRGFLGGAGTLALATASGLLLPGTAHAATTITTNQTGTDGMYYSFWTDGGGSVSMTLNGGGSYSTQWTNCGNFVAGKGWSTGGRRTVRYNGYFNPSGNGYGCLYGWTSNPLVEYYIVDNWGSYRPTGTYKGTVSSDGGTYDIYQTTRYNAPSVEGTKTFQQYWSVRQSKVTSGSGTITTGNHFDAWARAGMNMGQFRYYMIMATEGYQSSGSSNITVSG

SEQ ID NO: 100 - protein, 335 aa, Streptomyces coelicolor A3
  MISC_FEATURE (1)..(335): xylanase
MNLLVQPRRRRRGPVTLLVRSAWAVALAALAALMLPGTAQADTVVTTNQEGTNNGYYYSFWTDSQGTVSMNMGSGGQYSTSWRNTGNFVAGKGWANGGRRTVQYSGSFNPSGNAYLALYGWTSNPLVEYYIVDNWGTYRPTGEYKGTVTSDGGTYDIYKTTRVNKPSVEGTRTFDQYWSVRQAKRTGGTITTGNHFDAWARAGMPLGNFSYYMIMATEGYQSSGSSSINVGGTGGGDNGGGDNGGGGGGCTATVSAGQKWGDRYNLDVSVSGASDWTVTMNVPSPAKVLSTWNVNASYPSAQTLTARSNGSGNNWGATIQANGNWTWPSVSCSAG

SEQ ID NO: 101 - protein, 335 aa, Streptomyces lividans [sequence omitted]

SEQ ID NO: 102 - protein, 240 aa, Streptomyces lividans
  MISC_FEATURE (1)..(240): xylanase
MQQDGTQQDRIKQSPAPLNGMSRRGFLGGAGTLALATASGLLLPGTAHAATTITTNQTGTDGMYYSFWTDGGGSVSMTLNGGGSYSTQWTNCGNFVAGKGWSTGDGNVRYNGYFNPVGNGYGCLYGWTSNPLVEYYIVDNWGSYRPTGTYKGTVSSDGGTYDIYQTTRYNAPSVEGTKTFQQYWSVRQSKVTSGSGTITTGNHFDAWARAGMNMGQFRYYMIMATEGYQSSGSSNITVSG

SEQ ID NO: 103 - protein, 191 aa, Streptomyces olivaceoviridis E-86
  MISC_FEATURE (1)..(191): xylanase
ATVITTNQTGTNNGFYYSFWTDGGGSVSMTLNSGGNYSTSWTNCGNFAAGKGWSNGGRRNVQYSGSFYPSGNGYLALYGWTSNPLVEYYIVDNWGNYRPTGTYKGTVTSGGGTYDVYQTTRYNAPSVEGTKTFNQYWSVRQSKRTGGTITTGNHFDAWARYGMQLGSFSYYMILATEGYQSSGSSNITVSG

SEQ ID NO: 104 - protein, 240 aa, Streptomyces sp. EC3
  MISC_FEATURE (1)..(240): xylanase
MQQDGKRQDQNQQNPAPFSGLSRRGFLGGAGTVALATASGLLLPSTAHAATTITTNQTGYDGMYYSFWTDGGGSVSMTLNGGGSYSTQWTNCGNFVAGKGWGNGGRRTVRYSGYFNPSGNGYGCLYGWTSNPLVEYYIVDNWGSYRPTGEYRGTVYSDGGTYDIYKTTRYNAPSVEGTRTFDQYWSVRQSKVIGSGTITTGNHFDAWARAGMNLGQFQYYMIMATEGYQSSGSSNITVSG

SEQ ID NO: 105 - protein, 228 aa, Streptomyces sp. S38
  MISC_FEATURE (1)..(228): xylanase
MTKDNTPIRPVSRRGFIGRAGALALATSGLMLPGTARADTVITTNQTGTNNGYYYSFWTDGGGSVSMNLASGGSYGTSWTNCGNFVAGKGWANGARRTVNYSGSFNPSGNAYLTLYGWTANPLVEYYIVDNWGTYRPTGTYKGTVTSDGGTYDVYQTTRVNAPSVEGTKTFNQYWSVRQSKRTGGSITAGNHFDAWARYGMPLGSFNYYMIMATEGYQSSGSSSISVS

SEQ ID NO: 106 - protein, 335 aa, Streptomyces thermocyaneoviolaceus
  MISC_FEATURE (1)..(335): xylanase
MNTLVHPQGRAGGLRLLVRAAWALALAALAAMMFGGTARADTITSNQTGTHNGYFYSFWTDAPGTVTMNTGAGGNYSTQWSNTGNFVAGKGWATGGRRTVTYSGTFNPSGNAYLALYGWSQNPLVEYYIVDNWGTYRPTGTYKGTVYSDGGTYDIYMTTRYNAPSIEGTKTFNQYWSVRQNKRTGGTITTGNHFDAWAAHGMPLGTFNYMILATEGYQSSGSSNITVGDSGGDNGGGGGGGGGGGNTGGCTATLSAGEQWSDRYNLNVSVSGSDNWTVTMRVPAPEKVMATWNVTASYPDAQTLVARPNGNGNNWGVTIQKNGSTTWPTVSCSVG

SEQ ID NO: 107 - protein, 335 aa, Streptomyces thermoviolaceus OPC-520
  MISC_FEATURE (1)..(335): xylanase
MNTLVHPQGRAGGLRLLVRAAWALALAALAAMMVGGTARADTITSNQTGTHNGYFYSFWTDAPGTVTMNTGAGGNYSTQWSNTGNFVAGKGWATGGRRTVTYSGTFNPSGNAYLALYGWSQNPLVEYYIVDNWGTYRPTGTYKGTVYSDGGTYDIYMTTRYNAPSIEGTKTFNQYWSVRQNKRTGGTITTGNHFDAWAAHGMPLGTFNYMILATEGYQSSGSSNITVGDSGGDNGGGGGGGGGGGNTGGCTATLSAGEQWSDRYNLNVSVSGSDNWTVTMRVPAPEKVMATWNVTASYPDAQTLVARPNGNGNNWGVTIQKNGSTTWPTVSCSVG

SEQ ID NO: 108 - protein, 329 aa, Streptomyces viridosporus
  MISC_FEATURE (1)..(329): xylanase
MNAFAHPRGRRHGRSAPMSPRSTWAVLLAALAVMLLPGTATAAPVITTNQTGTNNGWWYSFWTDAQGTVSMDLGSGGTYSTQWRNTGNFVAGKGWSTGGRKTVNYSGTFNPSGNAYLTLYGWTTGPLIEYYIVDNWGTYRPTGKYKGTVTSDGGTYDIYKTTRYNAPSIEGTKTFDQYWSVRQSKRTGGTITSGNHFDAWARNGMNLGNHNYMIMATEGYQSSGSSTITVSESGSGGGGGGGGCTATLSAGEKWGDRYNLNVSVSGSSNWTVTMKVPSPAKVSSTWNVSTSYPDSQTLVAKPNGNGNNWGVTIQTNGNWTWPTVSCSVG

SEQ ID NO: 109 - protein, 338 aa, Thermobifida fusca
  MISC_FEATURE (1)..(338): xylanase
MNHAPASLKSRRRFRPRLLIGKAFAAALVAVVTMIPSTAAHAAVTSNETGYHDGYFYSFWTDAPGTVSMELGPGGNYSTSWRNTGNFVAGKGWATGGRRTVTYSASFNPSGNAYLTLYGWTRNPLVEYYIVESWGTYRPTGTYMGTVTTDGGTYDIYKTTRYNAPSIEGTRTFDQYWSVRQSKRTSGTITAGNHFDAWARHGMHLGTHDYMIMATEGYQSSGSSNVTLGTSGGGNPGGGNPPGGGNPPGGGGCTATLSAGQQWNDRYNLNVNVSGSNNWTVTVNVPWPARIIATWNIHASYPDSQTLVARPNGNGNNWGMTIMHNGNWTWPTVSCSAN

SEQ ID NO: 110 - protein, 225 aa, Thermomyces lanuginosus
  MISC_FEATURE (1)..(225): xylanase
MVGFTPVALAALAATGALAFPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 111 - protein, 220 aa, Trichoderma sp. SY
  MISC_FEATURE (1)..(220): xylanase
MVAFTSLLAGFAAVAGVLSAPTEDVQVEKRQVIGPGTGFNNGYYYSYWNDGHAGVTYTNGAGGSFSVNWANSGNFVGGKGWNPGSSTRTINFSGSYSPNDNSYLSVYGWSKNPLIEYYIVENFGTYNPSTGATKLGEVTLDGSVYDIYRTQRVNQPSIIGTATFYQYWSVRRSHRSSGSVNVGNHFNAWRNLGLTLGQLDYQIIAVEGYFSSGSANINVS

SEQ ID NO: 112 - protein, 223 aa, Trichoderma viride
  MISC_FEATURE (1)..(223): xylanase
MVSFTTLLAGFVAVTGVLSAPTETVEVVDVEKRQTIGPGTGFNNGYYYSYWNDGHSGVTYTNGAGGSFSVNWANSGNFVGGKGWNPGSSSRVINFSGSYNPNGNSYLSVYGWSKNPLIEYYIVENFGTYNPSTGTTKLGEVTSDGSVYDIYRTQRVNQPSIIGTATFYQYWSVRRNHAPAARSRLRTTSNAWRNLGLTLGTLDYQIIAVEGYFSSGNANINVS

SEQ ID NO: 113 - protein, 223 aa, Trichoderma viride YNUCCO183
  MISC_FEATURE (1)..(223): xylanase
MVSFTTLLAGFVAVTGVLSAPTENVEVVDVEKRQTIGPGTGFNNGYYYSYWNDGHSGVTYTNGAGGSFSVNWANSGNFVGGKGWNPGSSSRVINFSGSYNPNGNSYLSVYGWSKNPLIEYYIVENFGTYNPSTGTTKLGEVTSDGSVYDIYRTQRVNQPSIIGTATFYQYWSVRRNHRSSGSVTVANHFNAWRNLGLTLGTLDYQIIAVEGYFSSGNANINVS

SEQ ID NO: 114 - DNA, 4855 bp, Pichia pastoris
  misc_feature (1)..(4855): pTrcHis_Xy1A1A
tggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggcccttatggcttcgacagactactggcaaaattggactgatggtggtgggacagtaaatgctaccaatggatctgatggcaattacagcgtttcatggtcaaattgcgggaattttgttgttggtaaaggctggactaccggatcagcaactagggtaataaactataatgccggagccttttcgccgtccggtaatggatatttggctctttatgggtggacgagaaattcactcatagaatattacgtcgttgatagctgggggacttatagacctactggaacttataaaggcactgtgactagtgatggagggacttatgacatatacacgactacacgaaccaacgcaccttccattgacggcaataatacaactttcacccagttctggagtgttaggcagtcgaagagaccgattggtaccaacaataccatcacctttagcaaccatgttaacgcctggaagagtaaaggaatgaatttggggagtagttggtcttatcaggtattagcaacagagggctatcaaagtagtgggtactctaacgtaacggtctggtaaaagggcgaattcaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatc

SEQ ID NO: 115 - DNA, 4855 bp, Pichia pastoris
  misc_feature (1)..(4855): pTrcHis_Xy1A1A
tggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggcccttatggcttcgacagactactggcaaaattggactgatggtggtgggacagtaaatgctaccaatggatctgatggcaattacagcgtttcatggtcaaattgcgggaattttgttgttggtaaaggctggactaccggatcagcaactagggtaataaactataatgccggagccttttcgccgtccggtaatggatatttggctctttatgggtggacgagaaattcactcatagcttattacgtcgttgatagctgggggacttatagacctactggaacttataaaggcactgtgactagtgatggagggacttatgacatatacacgactacacgaaccaacgcaccttccattgacggcaataatacaactttcacccagttctggagtgttaggcagtcgaagagaccgattggtaccaacaataccatcacctttagcaaccatgttaacgcctggaagagtaaaggaatgaatttggggagtagttggtcttatcaggtattagcaacagagggctatcaaagtagtgggtactctaacgtaacggtctggtaaaagggcgaattcaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatc

SEQ ID NO: 116 - DNA, 4537 bp, Pichia pastoris
  misc_feature (1)..(4537): pCR4Blunt Xy1A1A_E79A
gtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcccttctcgagaaaagagcttcgacagactactggcaaaattggactgatggtggtgggacagtaaatgctaccaatggatctgatggcaattacagcgtttcatggtcaaattgcgggaattttgttgttggtaaaggctggactaccggatcagcaactagggtaataaactataatgccggagccttttcgccgtccggtaatggatatttggctctttatgggtggacgagaaattcactcatagcttattacgtcgttgatagctgggggacttatagacctactggaacttataaaggcactgtgactagtgatggagggacttatgacatatacacgactacacgaaccaacgcaccttccattgacggcaataatacaactttcacccagttctggagtgttaggcagtcgaagagaccgattggtaccaacaataccatcacctttagcaaccatgttaacgcctggaagagtaaaggaatgaatttggggagtagttggtcttatcaggtattagcaacagagggctatcaaagtagtgggtactctaacgtaacggtctggtaataggcggccgaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaat

SEQ ID NO: 117 - DNA, 8566 bp, Pichia pastoris
  misc_feature (1)..(8566): pPIC9 Xy1A1A_E79A
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagcttcgacagactactggcaaaattggactgatggtggtgggacagtaaatgctaccaatggatctgatggcaattacagcgtttcatggtcaaattgcgggaattttgttgttggtaaaggctggactaccggatcagcaactagggtaataaactataatgccggagccttttcgccgtccggtaatggatatttggctctttatgggtggacgagaaattcactcatagcttattacgtcgttgatagctgggggacttatagacctactggaacttataaaggcactgtgactagtgatggagggacttatgacatatacacgactacacgaaccaacgcaccttccattgacggcaataatacaactttcacccagttctggagtgttaggcagtcgaagagaccgattggtaccaacaataccatcacctttagcaaccatgttaacgcctggaagagtaaaggaatgaatttggggagtagttggtcttatcaggtattagcaacagagggctatcaaagtagtgggtactctaacgtaacggtctggtaataggcggccgaaagggcgaattcgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 118 - protein, 14 aa, Artificial Sequence
  environmental sample
TRNSLIEYYVVDSW

SEQ ID NO: 119 - DNA, 44 bp, Artificial Sequence
  environmental sample
ggacgagaaattcactcatagaatattacgtcgttgatagctgg

SEQ ID NO: 120 - protein, 14 aa, Artificial Sequence
  Xy1A1A modified
TRNSLIAYYVVDSW

SEQ ID NO: 121 - DNA, 44 bp, Artificial Sequence
  PCR primer
ggacgagaaattcactcatagcttattacgtcgttgatagctgg

SEQ ID NO: 122 - DNA, 44 bp, Artificial Sequence
  PCR primer
ccagctatcaacgacgtaataagctatgagtgaatttctcgtcc

SEQ ID NO: 123 - DNA, 46 bp, Artificial Sequence
  PCR primer
tttccctctcgagaaaagagcttcgacagactactggcaaaattgg

SEQ ID NO: 124 - DNA, 46 bp, Artificial Sequence
  PCR primer
ttttccttttgcggccgcctattaccagaccgttacgttagagtac

SEQ ID NO: 125 - DNA, 23 bp, Artificial Sequence
  Primer 5
gcttcttgctgtagaatttgggc

SEQ ID NO: 126 - DNA, 24 bp, Artificial Sequence
  Primer 6
ccaaagcggtcggacagtgctccg

SEQ ID NO: 127 - DNA, 29 bp, Artificial Sequence
  Primer 7
ggaattcgccttagacatgactgttcctc

SEQ ID NO: 128 - DNA, 27 bp, Artificial Sequence
  Primer 8
gttggccagtaaatatagagatcaagc

(19) United States
(12) Patent Application Publication
(10) Pub. No.: US 2011/0053195 A1
(43) Pub. Date: Mar. 3, 2011

(54) CATALYTICALLY INACTIVE PROTEINS AND METHOD FOR RECOVERY OF ENZYMES FROM PLANT-DERIVED MATERIALS

(75) Inventors: Michael William Bauer, Newton, MA (US); Justin DeFontes, Cary, NC (US)

(73) Assignee: SYNGENTA PARTICIPATIONS AG, Basel (CH)

(21) Appl. No.: 12/303,563

(22) PCT Filed: Jun. 12, 2007
(86) PCT No.: PCT/US07/71012

§ 371 Date: May 21, 2009

Related U.S. Application Data
(60) Provisional application No. 60/814,675, filed on Jun. 16, 2006.

(51) Int. Cl. C12Q 1/34; C12N 9/24; C07H 21/04; C12N 15/63; C12N 1/15; C12N 1/21; C12N 1/19

(52) U.S. Cl. 435/18; 435/200; 536/23.2; 435/320.1; 435/254.11; 435/252.3; 435/254.2; 435/254.23; 435/252.33; 435/252.31; 435/254.5; 435/254.3; 435/254.6; 435/254.9; 435/254.8

(57) ABSTRACT

An inactive xylanase molecule for the recovery of xylanase activity in plant-derived material containing active xylanase enzyme(s) and xylanase inhibitors. The inactive xylanase molecule of binds to xylanase inhibitors in the plant-derived material, thereby allowing accurate measurement of xylanase enzyme activity of the enzyme contained in the plant-derived material. The invention further includes amino acid molecules depicted by SEQ ID NOS. 4 through 112, wherein the catalytically active sites of each of the amino acids have been modified resulting in inactive xylanase molecules. A method of production of the inactive xylanase molecules includes expression of the inactive xylanase molecule in microbial or eukaryal (e.g., yeast including Pichia pastoris) host cell an expression cassette comprising a promoter operably linked to a nucleic acid molecule encoding the inactive xylanase molecule and using the expressed molecule in an assay to recover the xylanase enzyme activity in plant-derived material, for example, in plant-derived material such as animal feed.

### FIELD OF THE INVENTION

The present invention relates to mutated xylanase coding sequences to produce catalytically-inactive proteins. The invention further relates to the expression of these mutated xylanases in microbes and yeast. The invention also relates to the use of catalytically-inactive proteins to improve the recoverability of xylanase activity from plant-derived materials, such as formulated animal feed.

### BACKGROUND OF THE INVENTION

Xylans are linear polysaccharides formed from beta-1,4 -linked D-xylopyranoses. Xylans frequently contain side chains of alpha-1,2, alpha-1,3, or alpha-1,2 and alpha-1,3 linked L-arabinofuranoside. These substituted xylans are commonly referred to as arabinoxylans. Xylans and arabinoxylans are one of the main non-starch polysaccharides (NSPs) in plants. These NSPs form viscous solutions that can be problematic in baking, brewing, and animal feed applications. For example, during the preparation of doughs in baking applications, the presence of xylans and arabinoxylans results in sticky doughs that adhere to equipment and present fouling problems. In brewing applications, xylans and arabinoxylans increase the viscosity of wort thus negatively influencing its filterability, a potentially costly and time-consuming problem. In animal feed applications, non-starch polysaccharides (NSP) have been implicated in the variability of the nutritional quality of cereals for chickens, associated with changes in viscosity of digesta (Bedford, M. R. & H. L. Classen). In pulp and paper applications, xylans and lignins physically associated with them, bind to cellulose. Harsh bleaching chemicals are frequently used to remove the lignins and increase the whiteness of the cellulose.

Xylanase enzymes (e.g., endo-1,4-beta-xylanase, EC 3.2.1.8) break down non-starch polysaccharides in plants. In nature, plant pathogens such as fungi and bacterium produce xylanase enzymes to digest plant structural materials. Xylanases hydrolyze internal beta-1,4-xylosidic linkages in xylan to produce smaller molecular weight xylo-oligomers. Xylanases mainly belong to two glycoside hydrolase families, 10 and 11. Family 10 and 11 enzymes hydrolyze the xylan linkages by virtue of active site catalytic residues. The active site includes a nucleophile catalytic residue as well as an acid/base catalytic residue. For example, family 11 xylanases include a nucleophile catalytic residue corresponding to position 78 and an acid/base catalytic residue corresponding to position 172 of a Bacillus circulans xylanase. Other catalytic residues are known. It has also been shown that amino acid substitutions at these sites produces inactive enzymes. (Wararchuk et al; Lawson et al)

Xylanases are added to plant-derived materials used in numerous industrial applications. For example, xylanases are used in the processing and manufacturing human foods. Grains and flours destined for human foods can be enzymatically treated with xylanase to reduce the xylan content of the material. The reduced levels of xylan enhance the quality of the food by increasing the nutrient availability of essential minerals such as iron, calcium, and zinc. In addition to increasing the nutritional quality of food, xylanase used during food processing can improve the overall efficiency of the food production method.

Addition of xylanase to wort improves fermentation in the brewing industry. Xylanases are also added to paper pulp in the paper bleaching process to degrade xylans and improve paper brightness.

Xylanase enzymes may also be used advantageously in monogastrics as well as in polygastrics, especially young calves. Diets for fish and crustaceans may also be supplemented with xylanase enzymes to further improve feed conversion ratio. Feed supplemented with xylanase enzymes may also be provided to animals such as poultry, e.g., turkeys, geese, ducks, as well as swine, equine, bovine, ovine, caprine, canine and feline, as well as fish and crustaceans. When added to animal feeds (e.g. for monogastric animals, including poultry or swine) that contain cereals (e.g. barley, wheat, maize, rye, triticale or oats) or cereal by-products, xylanase enzymes improve the break-down of plant cell walls leading to increased utilization of the plant nutrients by the animal. This leads to improved growth rate and feed conversion. Also, the viscosity of the feeds containing xylan can be reduced by the presence of xylanase enzyme.

For animal feed, the increase in apparent metabolizable energy due to xylanase supplementation is difficult to predict. Current technologies do not accurately determine xylanase activity in animal feed. Accurate recovery of xylanase activity is necessary to consistently optimise animal feed formulation.

Several factors likely contribute to difficulty in recovering xylanase activity including physical binding of enzyme to components of the plant material (e.g., cellulosic or hemicellulosic polysaccharides), inhibition by salts or heavy metals, inhibition by endogenous xylanase inhibitors, or degradation by endogeneous plant proteases. The problem can be worsened in certain applications (e.g., animal feed) where the inclusion level of the xylanase enzyme is very low (e.g., ppb or ppm). For animal feed applications, accurate determination of xylanase activity, “xylanase recovery,” is difficult. Most commercial xylanases designed for feed applications were not chosen due to poor recoverability of their enzymatic activity from formulated feed. The problem can be especially acute with recoveries of some enzymes being only 10-20%.

There is a need, therefore, to develop compositions and methods to improve the recovery of xylanase activity in various industrial applications such as animal feed and grain processing, biofuels, cleaning, fabric care, chemicals, plant processing, delignifying and brightening of pulp and paper and others.

### SUMMARY OF THE INVENTION

The present invention includes an inactive xylanase molecule used in a novel method for the recovery of xylanase activity in a plant derived material containing active xylanase enzyme(s). The inactive xylanase of the present invention is capable to binding xylanase inhibitors in a plant-derived material, thereby allowing the method of the invention to measure activity of enzymatically functional xylanase in the plant-derived material, such as a feed formulation.

The present invention also includes a method for assessing the quality of xylanase enzymes contained in materials, such as animal feed, pulp, wort. In addition, the present invention includes a method for establishing the comparative value of xylanase activity across all such materials.

The present invention further includes a method for recovering the activity of a xylanase enzyme from plant derived materials, such as feed formulations, containing putative xylanase inhibitor(s) comprising the steps of providing an inactive xylanase molecule capable of binding to a xylanase inhibitor molecules, mixing the inactive xylanase molecule with a material comprising an active xylanase enzyme and the putative xylanase inhibitor under conditions sufficient for the inactive xylanase molecule and the xylanase inhibitors to bind together directly or indirectly, and measuring the activity of the xylanase enzyme.

The present invention further provides xylanases comprising SEQ ID NOS. 3 through 113, wherein when the catalytically active sites of the enzymes are modified inactive xylanase molecules are produced.

The invention also provides methods of preparing a catalytically-inactive xylanase protein, comprising the steps of: expression in a microbial or eukaryal (e.g., yeast including Pichia pastoris) host cell an expression cassette comprising a promoter operably linked to a nucleic acid molecule encoding a mutated xylanase which displays less than 0.1% of the activity of wild-type protein assayed under the identical conditions. The invention further provides methods of extracting an animal feed utilizing a buffer or solution comprising a mutated catalytically-inactive xylanase.

Also, the invention provides methods of improving the recovery of xylanase enzyme activity from feeds comprising the use of buffers or solutions containing a catalytically inactive xylanase.

The invention further includes a modified xylanase polypeptide, wherein the modification is at amino acid residue number 78 of the amino acid sequence depicted by SEQ ID NO. 3 or the equivalent position in other homologous xylanase polypeptides, wherein said modified xylanase polypeptide is inactive yet retains its ability to bind to xylanase inhibitors.

The invention also includes a modified xylanase polypeptide, wherein the modification is at amino acid residue number 78 of the amino acid sequence depicted by SEQ ID NO. 3 or equivalent position in a class 11 xylanase polypeptide.

The invention provides a modified xylanase polypeptide, wherein the modification is at amino acid residue number 78 of amino acid sequence depicted by SEQ ID NO. 3 or equivalent position in a xylanase amino acid sequence depicted by SEQ ID NOS. 4 through 114.

The invention also provides an isolated nucleic acid molecule encoding the modified xylanase polypeptide.

The invention also includes an expression cassette comprising a nucleic acid molecule encoding an inactive xylanase protein.

### BRIEF DESCRIPTION OF THE DRAWINGS

FIG. 1 is the vector map of plasmid pTrcHis_Xy1A1A.

FIG. 2 is the vector map of plasmid pTrcHis_Xy1A1_E79A.

FIG. 3 is the vector map of plasmid pCR4Blunt Xy1A1A_E79A.

FIG. 4 is the vector map of plasmid pIC9 Xy1A1A_E79A.

FIG. 5 is a table that shows the alignment of amino acid sequences of xylanase enzymes SEQ ID NO. 3 through SEQ ID NO. 113.

### BRIEF DESRIPTION OF THE SEQUENCE LISTING

SEQ ID NO. 1 is the nucleotide sequence of coding region of the Xy1A1A_E79A gene.

SEQ ID NO. 2 is the amino acid sequence of the Xy1A1A_E79A gene.

SEQ ID NO. 3 is the nucleotide sequence of the Xy1A1A-xylanase gene

SEQ ID NO. 4 is the amino acid sequence of the xylanase Aeromonas punctata ME-1 gene.

SEQ ID NO. 5 is the amino acid sequence of the xylanase Ascochyta pisi gene.

SEQ ID NO. 6 is the amino acid sequence of the xylanase Ascochyta rabiei gene.

SEQ ID NO. 7 is the amino acid sequence of the xylanase Aspergillus aculeatus gene.

SEQ ID NO. 8 is the amino acid sequence of the xylanase Aspergillus awamori ATCC11358 gene.

SEQ ID NO. 9 is the amino acid sequence of the xylanase Aspergillus cf. niger BCC14405 gene.

SEQ ID NO. 10 is the amino acid sequence of the xylanase Aspergillus kawachii gene.

SEQ ID NO. 11 is the amino acid sequence of the xylanase Aspergillus kawachii IFO4308 gene.

SEQ ID NO. 12 is the amino acid sequence of the xylanase Aspergillus nidulans FGSC A4 gene.

SEQ ID NO. 13 is the amino acid sequence of the xylanase Aspergillus niger gene.

SEQ ID NO. 14 is the amino acid sequence of the xylanase Aspergillus niger gene.

SEQ ID NO. 15 is the amino acid sequence of the xylanase Aspergillus niger gene.

SEQ ID NO. 16 is the amino acid sequence of the xylanase Aspergillus niger IFO4066 gene.

SEQ ID NO. 17 is the amino acid sequence of the xylanase Aspergillus oryzae gene.

SEQ ID NO. 18 is the amino acid sequence of the xylanase Aspergillus oryzae gene.

SEQ ID NO. 19 is the amino acid sequence of the xylanase Aspergillus tubigensis gene.

SEQ ID NO. 20 is the amino acid sequence of the xylanase Aureobasidium pullulans var. melanigenum.

SEQ ID NO. 21 is the amino acid sequence of the xylanase Auerobasidium pullulans gene.

SEQ ID NO. 22 is the amino acid sequence of the xylanase Bacillus agaradhaerens AC13 gene.

SEQ ID NO. 23 is the amino acid sequence of the xylanase Bacillus circulans gene.

SEQ ID NO. 24 is the amino acid sequence of the xylanase Bacillus firmus gene.

SEQ ID NO. 25 is the amino acid sequence of the xylanase Bacillus firmus K-1 gene.

SEQ ID NO. 26 is the amino acid sequence of the xylanase Bacillus halodurans C-125 gene.

SEQ ID NO. 27 is the amino acid sequence of the xylanase Bacillus pumilus gene.

SEQ ID NO. 28 is the amino acid sequence of the xylanase Bacillus pumilus HB030 gene.

SEQ ID NO. 29 is the amino acid sequence of the xylanase Bacillus sp. gene.

SEQ ID NO. 30 is the amino acid sequence of the xylanase Bacillus sp. YA-14 gene.

SEQ ID NO. 31 is the amino acid sequence of the xylanase Bacillus sp. YA-335 gene.

SEQ ID NO. 32 is the amino acid sequence of the xylanase Bacillus subtilis B230 gene.

SEQ ID NO. 33 is the amino acid sequence of the xylanase Bacillus subtilis subsp. subtilis str. 168 gene.

SEQ ID NO. 34 is the amino acid sequence of the xylanase Caldicellulosiruptor sp. Rt69B.1 gene.

SEQ ID NO. 35 is the amino acid sequence of the xylanase Cellulomonas fimi gene.

SEQ ID NO. 36 is the amino acid sequence of the xylanase Cellulomonas pachnodae gene.

SEQ ID NO. 37 is the amino acid sequence of the xylanase Cellvibrio japonicus gene.

SEQ ID NO. 38 is the amino acid sequence of the xylanase Cellvibrio mixtus gene.

SEQ ID NO. 39 is the amino acid sequence of the xylanase Chaetomium gracile gene.

SEQ ID NO. 40 is the amino acid sequence of the xylanase Chaetomium gracile gene.

SEQ ID NO. 41 is the amino acid sequence of the xylanase Chaetomium thermophilum gene.

SEQ ID NO. 42 is the amino acid sequence of the xylanase Chaetomium thermophilum gene.

SEQ ID NO. 43 is the amino acid sequence of the xylanase Chaetomium thermophilum gene.

SEQ ID NO. 44 is the amino acid sequence of the xylanase Claviceps purpurea gene.

SEQ ID NO. 45 is the amino acid sequence of the xylanase Clostridium cellulovorans gene.

SEQ ID NO. 46 is the amino acid sequence of the xylanase Clostridium saccharobutylicum P262 gene.

SEQ ID NO. 47 is the amino acid sequence of the xylanase Clostridium stercorarium F-9 gene.

SEQ ID NO. 48 is the amino acid sequence of the xylanase Clostridium thermocellum F1/YS gene.

SEQ ID NO. 49 is the amino acid sequence of the xylanase Clostridium thermocellum F1/YS gene.

SEQ ID NO. 50 is the amino acid sequence of the xylanase Cochliobolus carbonum gene.

SEQ ID NO. 51 is the amino acid sequence of the xylanase Cochliobolus carbonum gene.

SEQ ID NO. 52 is the amino acid sequence of the xylanase Cochliobolus carbonum gene.

SEQ ID NO. 53 is the amino acid sequence of the xylanase Cochliobolus sativus gene.

SEQ ID NO. 54 is the amino acid sequence of the xylanase Cryptococcus sp. S-2 gene.

SEQ ID NO. 55 is the amino acid sequence of the xylanase Dictyoglomus thermophilum Rt46B.1 gene.

SEQ ID NO. 56 is the amino acid sequence of the xylanase Emericella nidulans gene.

SEQ ID NO. 57 is the amino acid sequence of the xylanase Fibrobacter succinogenes gene.

SEQ ID NO. 58 is the amino acid sequence of the xylanase Fusarium oxysporum f. sp. Lycopersici gene.

SEQ ID NO. 59 is the amino acid sequence of the xylanase Fusarium oxysporum f. sp. Lycopersici gene.

SEQ ID NO. 60 is the amino acid sequence of the xylanase Geobacillus stearothermophilus No.236 gene.

SEQ ID NO. 61 is the amino acid sequence of the xylanase Gibberella zeae 180378 gene.

SEQ ID NO. 62 is the amino acid sequence of the xylanase Helminthosporium turcicum gene.

SEQ ID NO. 63 is the amino acid sequence of the xylanase Humicola grisea var. thermoidea 60849 gene.

SEQ ID NO. 64 is the amino acid sequence of the xylanase Humicola insolens gene.

SEQ ID NO. 65 is the amino acid sequence of the xylanase Hypocrea jecorina gene.

SEQ ID NO. 66 is the amino acid sequence of the xylanase Hypocrea jecorina gene.

SEQ ID NO. 67 is the amino acid sequence of the xylanase Hypocrea lixii E58 gene.

SEQ ID NO. 68 is the amino acid sequence of the xylanase Lentinula edodes Stamets CS-2 gene.

SEQ ID NO. 69 is the amino acid sequence of the xylanase Magnaporthe grisea gene.

SEQ ID NO. 70 is the amino acid sequence of the xylanase Neocallimastix frontalis gene.

SEQ ID NO. 71 is the amino acid sequence of the xylanase Neocallimastix patriciarum gene.

SEQ ID NO. 72 is the amino acid sequence of the xylanase Neocallimastix patriciarum gene.

SEQ ID NO. 73 is the amino acid sequence of the xylanase Neocallimastix patriciarum MCH3 gene.

SEQ ID NO. 74 is the amino acid sequence of the xylanase Neurospora crassa OR74A gene.

SEQ ID NO. 75 is the amino acid sequence of the xylanase Neurospora crassa OR74A gene.

SEQ ID NO. 76 is the amino acid sequence of the xylanase Nonomuraea flexuaosa gene.

SEQ ID NO. 77 is the amino acid sequence of the xylanase Orpinomyces sp. PC-2 gene.

SEQ ID NO. 78 is the amino acid sequence of the xylanase Paecilomyces varioti Bainier gene.

SEQ ID NO. 79 is the amino acid sequence of the xylanase Penicillium funiculosum gene.

SEQ ID NO. 80 is the amino acid sequence of the xylanase Penicillium funiculosum gene.

SEQ ID NO. 81 is the amino acid sequence of the xylanase Penicillium purpurogenum gene.

SEQ ID NO. 82 is the amino acid sequence of the xylanase Phaedon cochleariae gene.

SEQ ID NO. 83 is the amino acid sequence of the xylanase Phanerochaete chrysosporium ME446 gene.

SEQ ID NO. 84 is the amino acid sequence of the xylanase Pichia stipitis gene.

SEQ ID NO. 85 is the amino acid sequence of the xylanase Piromyces sp. gene.

SEQ ID NO. 86 is the amino acid sequence of the xylanase Polyplastron mutivesiculatum gene.

SEQ ID NO. 87 is the amino acid sequence of the xylanase Pseudomonas sp. ND137 gene.

SEQ ID NO. 88 is the amino acid sequence of the xylanase Ruminococcus albus gene.

SEQ ID NO. 89 is the amino acid sequence of the xylanase Ruminococcus albus gene.

SEQ ID NO. 90 is the amino acid sequence of the xylanase Ruminococcus flavefaciens 17 gene.

SEQ ID NO. 91 is the amino acid sequence of the xylanase Ruminococcus flavefaciens 17 gene.

SEQ ID NO. 92 is the amino acid sequence of the xylanase Ruminococcus flavefaciens 17 gene.

SEQ ID NO. 93 is the amino acid sequence of the xylanase Ruminococcus flavefaciens 17 gene.

SEQ ID NO. 94 is the amino acid sequence of the xylanase Ruminococcus sp. gene.

SEQ ID NO. 95 is the amino acid sequence of the xylanase Schizophyllum commune gene.

SEQ ID NO. 96 is the amino acid sequence of the xylanase Scytalidium acidophilum gene.

SEQ ID NO. 97 is the amino acid sequence of the xylanase Scytalidium thermophilum Af101-3 gene.

SEQ ID NO. 98 is the amino acid sequence of the xylanase Setosphaeria turcica gene.

SEQ ID NO. 99 is the amino acid sequence of the xylanase Streptomyces coelicolor A3 gene.

SEQ ID NO. 100 is the amino acid sequence of the xylanase Streptomyces coelicolor A3 gene.

SEQ ID NO. 101 is the amino acid sequence of the xylanase Streptomyces lividans gene.

SEQ ID NO. 102 is the amino acid sequence of the xylanase Streptomyces lividans gene.

SEQ ID NO. 103 is the amino acid sequence of the xylanase Streptomyces olivaceoviridis E-86 gene.

SEQ ID NO. 104 is the amino acid sequence of the xylanase Streptomyces sp. EC3 gene.

SEQ ID NO. 105 is the amino acid sequence of the xylanase Streptomyces sp. S38 gene.

SEQ ID NO. 106 is the amino acid sequence of the xylanase Streptomyces thermocyaneoviolaceus gene.

SEQ ID NO. 107 is the amino acid sequence of the xylanase Streptomyces thermoviolaceus OPC-520 gene.

SEQ ID NO. 108 is the amino acid sequence of the xylanase Streptomyces viridosporus gene.

SEQ ID NO. 109 is the amino acid sequence of the xylanase Thermobifida fusca gene.

SEQ ID NO. 110 is the amino acid sequence of the xylanase Thermomyces lanuginosus gene.

SEQ ID NO. 111 is the amino acid sequence of the xylanase Trichoderma sp. SY gene.

SEQ ID NO. 112 is the amino acid sequence of the xylanase Trichoderma viride gene.

SEQ ID NO. 113 is the amino acid sequence of the xylanase Trichoderma viride YNUCC0183 gene.

SEQ ID NO. 114 is the nucleotide sequence of plasmid pTrcHis_Xy1A1A

SEQ ID NO. 115 is the nucleotide sequence of plasmid pTRcHis_Xy1A1A_E79A

SEQ ID NO. 116 is the nucleotide sequence of plasmid pCR4Blunt Xy1A1A_E79A

SEQ ID NO. 117 is the nucleotide sequence of plasmid pPIC9 Xy1A1A_E79A.

SEQ ID NO. 118 is the amino acid sequence of Xy1A1A.

SEQ ID NO. 119 is the nucleotide sequence of Xy1A1A.

SEQ ID NO. 120 is the amino acid sequence of Xy1A1A_E79A

SEQ ID NO. 121 is the nucleotide sequence of Primer 1.

SEQ ID NO. 122 is the nucleotide sequence of Primer 2.

SEQ ID NO. 123 is the nucleotide sequence of Primer 3.

SEQ ID NO. 124 is the nucleotide sequence of Primer 4.

SEQ ID NO. 125 is the nucleotide sequence of Primer 5.

SEQ ID NO. 126 is the nucleotide sequence of Primer 6.

SEQ ID NO. 127 is the nucleotide sequence of Primer 7.

SEQ ID NO. 128 is the nucleotide sequence of Primer 8.

### DETAILED DESCRIPTION OF THE INVENTION

The present invention relates to the use of a catalytically-inactive xylanase or xylanases, as an additive to buffers or solutions used to extract plant-derived materials such as, pulp, wort, and human and animal feed or feedstuff that contains a xylanase enzyme.

The invention also includes a composition and method for improving the recovery of xylanase activity from plant derived materials containing a xylanase or xylanases.

The invention also includes a nucleic acid molecule (i.e., a polynucleotide) that encodes a catalytically inactive xylanase.

An “active xylanase” refers to a xylanase protein in its normal wild-type conformation, e.g., a catalytically active state, as opposed to an inactive state. The active state allows the protein to function normally. An active site is an available wild-type conformation at a site that has biological activity, such as the catalytic site of an enzyme, a cofactor-binding site, the binding site of a receptor for its ligand, and the binding site for protein complexes, for example.

The nucleic acid molecules that encode wild-type xylanase enzymes may be obtained from various organisms, including fungi and bacteria. The Brief Description of the Sequence. Listing sets forth amino acid sequences of family 11 xylanase enzymes (SEQ ID NOS. 4-113), wherein according to the invention, modification of their catalytic residues can result in inactive xylanase proteins.

An inactive state of a xylanase enzyme of the invention may result from denaturation, inhibitor binding, either covalently or non-covalently, mutation, secondary processing, e.g., phosphorylation or dephosphorylation of the nucleophile and/or acid/base catalytic residues of the corresponding xylanase enzyme. Inactive xylanase molecules of the invention may also be obtained by adding one or more amino acids into the xylanase polypeptide sequence, deletion one or more amino acid residues from its polypeptide sequence, extending polypeptide chain at either terminus and converting it to zymogen-like form, circular permutation of xylanase polypeptide sequence and other protein engineering methods. Simple modification of the polypeptide sequence can be carried out using numerous standard techniques such as site directed mutagenesis.

It is also within the scope of the present invention to knock out xylanase activity by using small molecule inhibitors including mechanism-based irreversible inhibitors. Gloster et al (2003) Chem Commun (Camb). (8):944-5. Ziser et al (1995) Carbohydr Res. 274:137-53. Other methods known to those skilled in the art and methods not yet known for inactivating xylanase enzymes are within the scope of the present invention. For present purposes, the term “modified” refers to xylanase enzymes that have been rendered catalytically inactive. Xylanase enzymes that are rendered inactive are also referred to herein as “inactive xylanase proteins” or “inactive xylanase molecules.”

An inactive xylanase protein of the present invention includes a xylanase protein that may have less than 0.1% active of the specific activity at about 37° C. compared with the wild type protein and which retains the ability to interact with xylanase inhibitors. In another embodiment, the inactive xylanase protein of the invention retains less than 0.01% of the specific activity of the wild-type protein and yet retains the ability to interact with xylanase inhibitors. In a further embodiment of the invention, the inactive xylanase retains less than 1% of the specific activity of the wild-type protein still retaining the ability to interact with xylanase inhibitors.

The present invention includes modified xylanase that is inactive in the absence of glycosylation. Alternatively, the present invention includes expressing an inactive xylanase protein that is glycosylated by the host.

The method of the present invention includes a microbial host cell an expression cassette comprising a promoter operably linked to a nucleic acid molecule encoding a catalytically inactive xylanase molecule. The microbial host cell may be a prokaryotic cell, such as a bacterial cell (e.g., Escherichia, Pseudomonas, Lactobacillus, and Bacillus), yeast (e.g., Saccharomyces, Schizosaccharomyces, Pichia or Hansenula) or fungal (e.g., Aspergillus or Trichoderma) cell. In one embodiment of the invention, the host cell is Pichia pastoris.

The invention also includes an inactive xylanase molecule that retains its ability to bind to xylanase inhibitors.

The invention further comprises a polynucleotide encoding the mutated, inactive xylanase operably linked to at least one regulatory sequence, such as a promoter, an enhancer, an intron, a termination sequence, or any combination thereof, and, optionally, to a second polynucleotide encoding a signal sequence, which directs the enzyme encoded by the first polynucleotide to a particular cellular location e.g., an extracellular location. Promoters can be constitutive promoters or inducible (conditional) promoters. As described herein, mutagenesis of a parent polynucleotide encoding a xylanase was employed to prepare variant (synthetic) DNAs encoding a mutated, catalytically-inactive xylanase molecule having impaired biochemical properties relative to the xylanase encoded by the parent polynucleotide, and wherein the inactive xylanase retains its ability to bind to xylanase inhibitors. In an embodiment of the present invention, mutated, catalytically-inactive xylanase molecules are screened for loss of activity at conditions of pH and temperature where the parent xylanase would have activity, unaltered or improved binding to xylanase inhibitors, or improved recovery of xylanase from solutions containing xylanase inhibitors. In another embodiment, the mutations in a number of the variant DNAs were combined to prepare a synthetic polynucleotide encoding a mutated, catalytically-inactive xylanase molecule with enhanced xylanase inhibitor binding and having a specific activity less than 0.1% relative to the xylanase encoded by the parent polynucleotide.

A wild-type xylanase polynucleotide may be obtained from any source including plant, bacterial or fungal nucleic acid, and any method may be employed to prepare a synthetic polynucleotide of the invention from a selected wild-type polynucleotide, e.g., combinatorial mutagenesis, recursive mutagenesis and/or DNA shuffling.

Thus, in one embodiment of the invention, the mutated xylanase has one or more amino acid substitutions relative to a wild-type xylanase, which substitutions are associated with the reduction of activity by greater than 99% relative to the parent xylanase at the temperatures and pHs when assayed under the same conditions. In an another embodiment of the invention, the mutated xylanase has one or more amino acid substitutions relative to a wild-type xylanase, which substitutions are associated with the reduction of activity by greater than 99.9% relative to the wild-type xylanase at the temperatures and pHs when assayed under the same conditions. In a further embodiment of the invention, the mutated xylanase has one or more amino acid substitutions relative to a wild-type xylanase, which substitutions are associated with the reduction of activity by greater than 99.99% relative to the wild-type xylanase at the temperatures and pHs when assayed under the same conditions.

In another embodiment, the mutated, catalytically-inactive xylanase has a specific activity less than 0.1% of the wild-type, or a specific activity less than 0.01% of the wild-type, or less than 0.001% activity of the wild-type, and which has a specific activity of less than 1.0 U/mg, more preferably less than 0.1 U/mg, and most preferably less than 0.01 U/mg at 37° C. and pH 5.0-5.5. One xylanase unit (XU) is the quantity of enzyme that liberates 1 μmol of reducing ends (xylose equivalents) per minute from WAXY (wheat arabinoxylan) at 37° C., pH 5.3, under standard conditions.

The invention also provides recombinant host cells comprising at least one of the nucleotide sequences that encode proteins amino acid molecules of SEQ ID NOS: 4 through 113, wherein one or more of the catalytic active site residues of the protein are inactivated. The recombinant host cell can be a bacteria, yeast or fungal cell. In particular the host cell is Escherichia, Pseudomonas, Lactobacillus, Bacillus, Saccharomyces, Schizosaccharomyces, Pichia, Hansenula, Aspergillus or Trichoderma cell. In one embodiment, the host cell is Pichia pastoris. In another embodiment of the invention, the vector of the present invention comprises pTrcHis_Xy1A1A_E79A (SEQ ID NO. 114) and/or pPIC9 Xy1A1A_E79A (SEQ ID NO. 117).

The invention also provides modified, catalytically-inactive xylanase formulations or formulated enzyme mixtures. The enzyme formulations further comprise a stabilizing compound, such as but not limited to sorbitol. The mutated, inactive xylanase molecule or formulations thereof may be added as a supplement to recover xylanase activity from plant derived materials, such as human food or beverage or animal feed or from components of food, beverage, and feed prior to, during, or after processing.

In one embodiment, the inactive xylanase of the invention is added to a mixture of feed components to improve the recoverability of xylanase that has been added prior to and/or following heat (e.g., steam) conditioning in a pellet mill.

Further provided is a method of preparing a catalytically-inactive xylanase containing composition for feed formulation prepared by combining a liquid solution comprising the inactive xylanase molecule of the invention and meal flour, e.g., soy meal flour, to yield a mixture; and drying the mixture to yield a dried composition. Drying the mixture may be accomplished by techniques routinely used in the art, including but not limited to lyophilising and/or heating.

The inactive xylanase molecule of the invention, as well as the enzyme mixtures described above, can be added to all feedstuffs containing xylanase to improve the recovery of the xylanase activity. Suitable and preferred examples are those that comply with the provisions of the feedstuffs legislation, such as premixes, complete feed, supplementary feed and mineral feed.

Inactive xylanases of the present invention can be used in any application for which xylanases are used, such as but not limited to, grain processing, biofuels, cleaning, fabric care, chemicals, plant processing, and delignifying and brightening of pulp and paper.

The construction of vectors which may be employed in conjunction with the present invention will be known to those of skill of the art in light of the present disclosure (see, e.g., Sambrook et al., Molecular Cloning, Cold Spring Harbor Press, 1989; Gelvin et al., Plant Molecular Biology Manual, 1990). The expression cassette of the invention may contain one or a plurality of restriction sites allowing for placement of the polynucleotide encoding a xylanase under the regulation of a regulatory sequence. The expression cassette may also contain a termination signal operably linked to the polynucleotide as well as regulatory sequences required for proper translation of the polynucleotide. The expression cassette containing the polynucleotide of the invention may be chimeric, meaning that at least one of its components is heterologous with respect to at least one of the other components. Expression of the polynucleotide in the expression cassette may be under the control of a constitutive promoter, inducible promoter, regulated promoter, viral promoter or synthetic promoter.

A variety of techniques are available and known to those skilled in the art for introduction of constructs into a cellular host. Transformation of microbial cells may be accomplished through use of polyethylene glycol, calcium chloride, viral infection, DEAE dextran, phage infection, electroporation and other methods known in the art. Transformation of fungus, in particular Pichia, may be accomplished according to “Pichia Protocols”, in Methods Mol. Biol., Higgins, David R.and Cregg, James M.; Eds. (Humana, Totowa, N.J.) (1998). Introduction of the recombinant vector into yeasts can be accomplished by methods including electroporation, use of spheroplasts, lithium acetate, and the like. Any method capable of introducing DNA into animal cells can be used: for example, electroporation, calcium phosphate, lipofection and the like.

### EXAMPLE 1

### Site-Directed Mutagenesis to Change the Catalytic Nucleophile of a Xylanase from Glutamic Acid to Alanine, Producing a Catalytically-Inactive Protein.

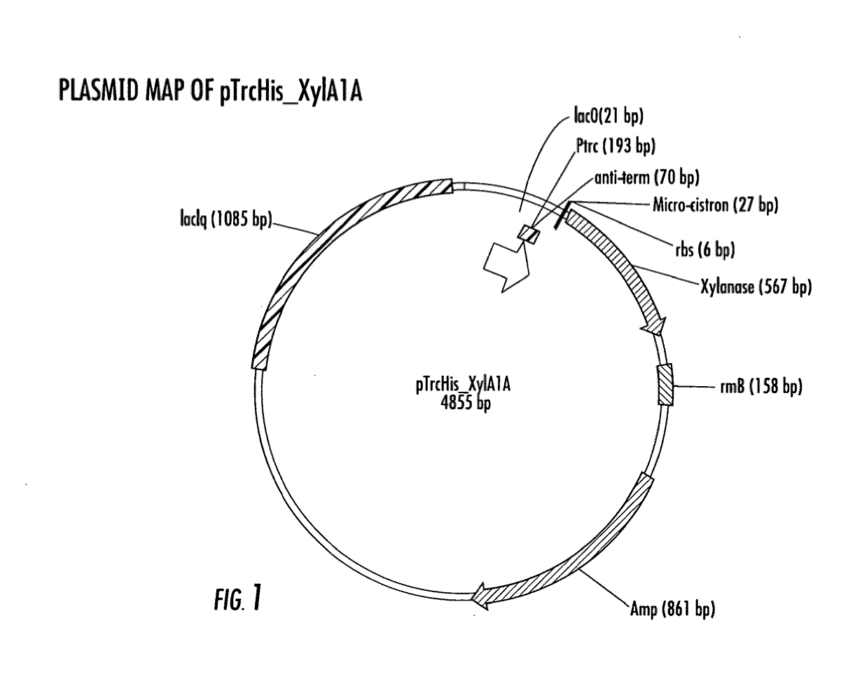

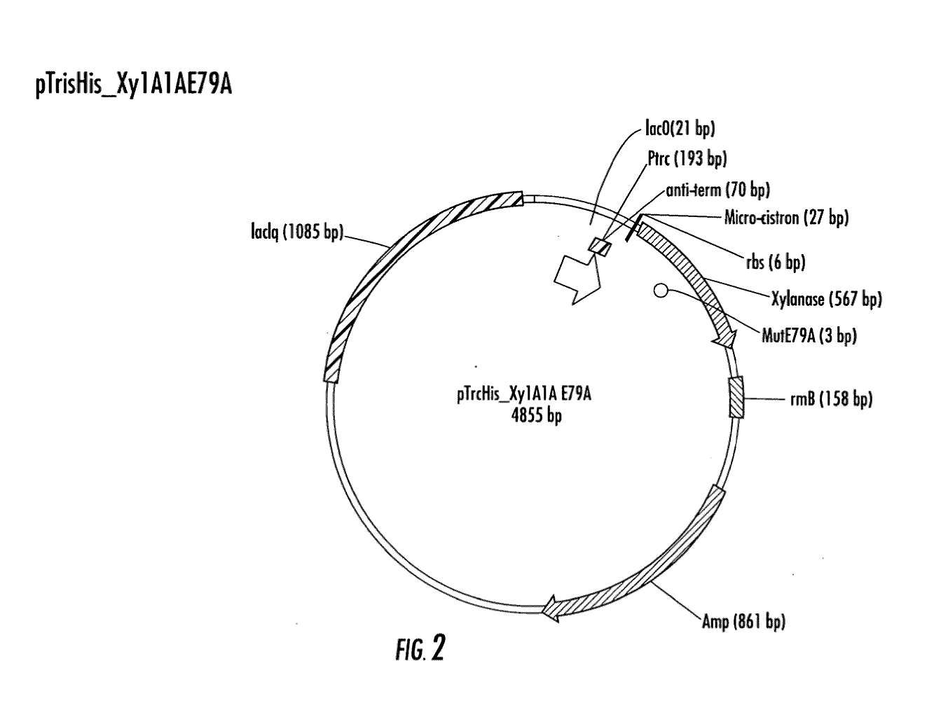

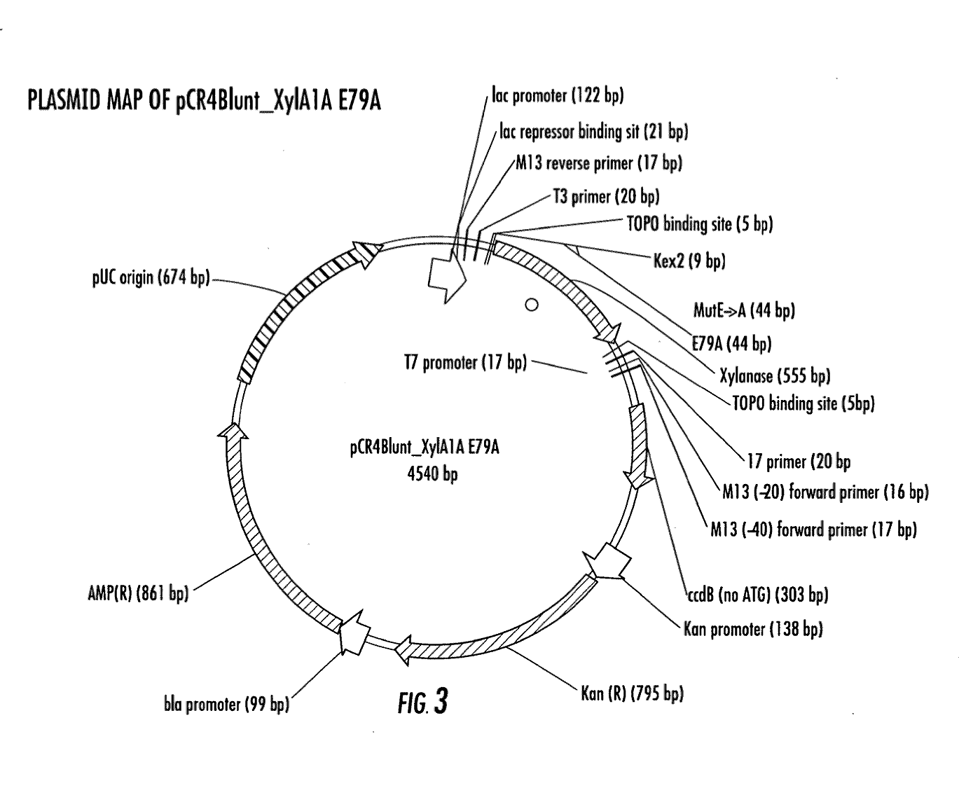

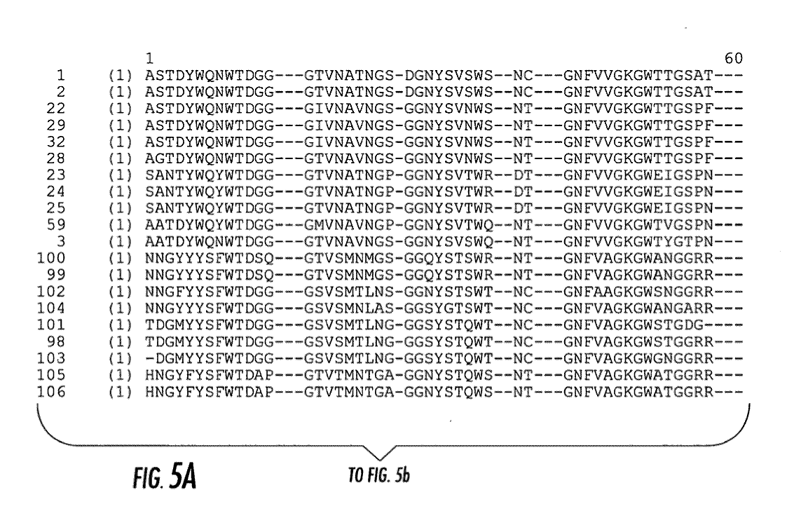

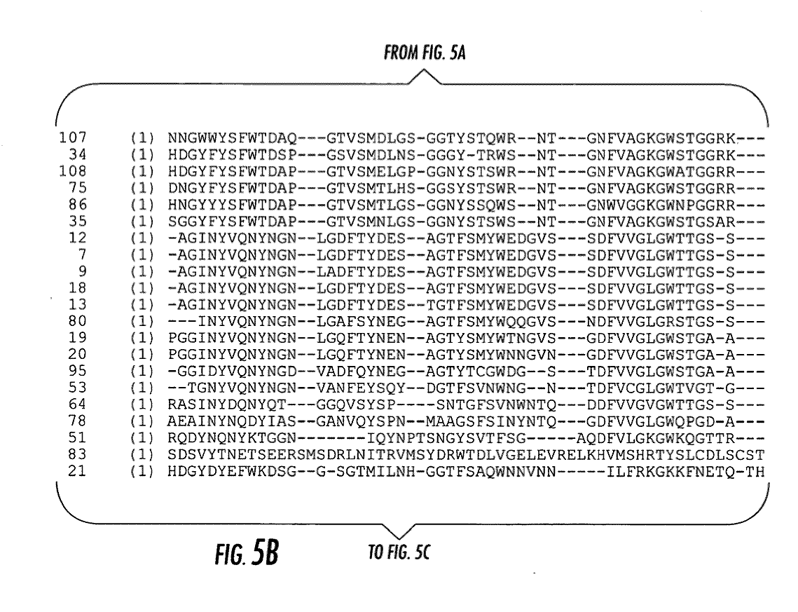

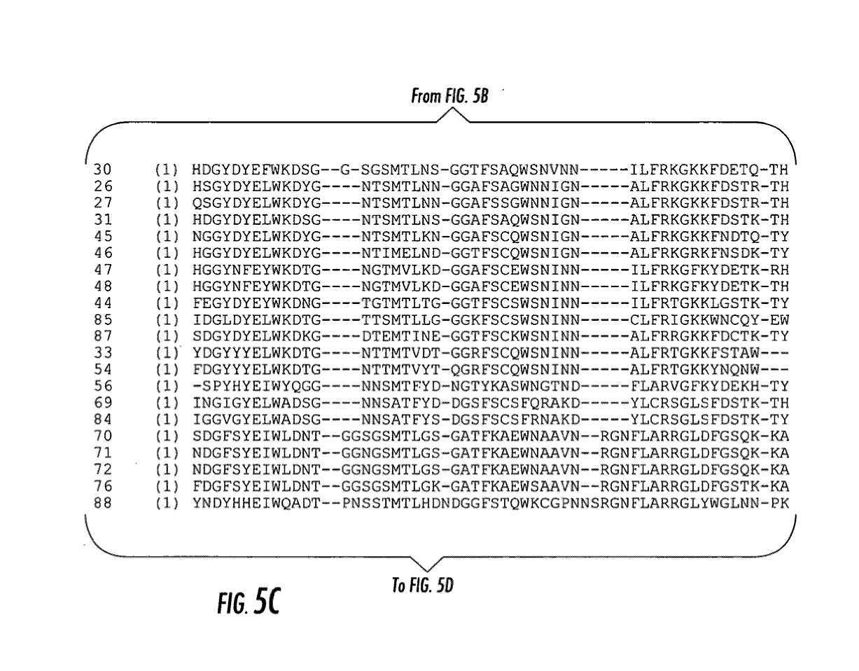

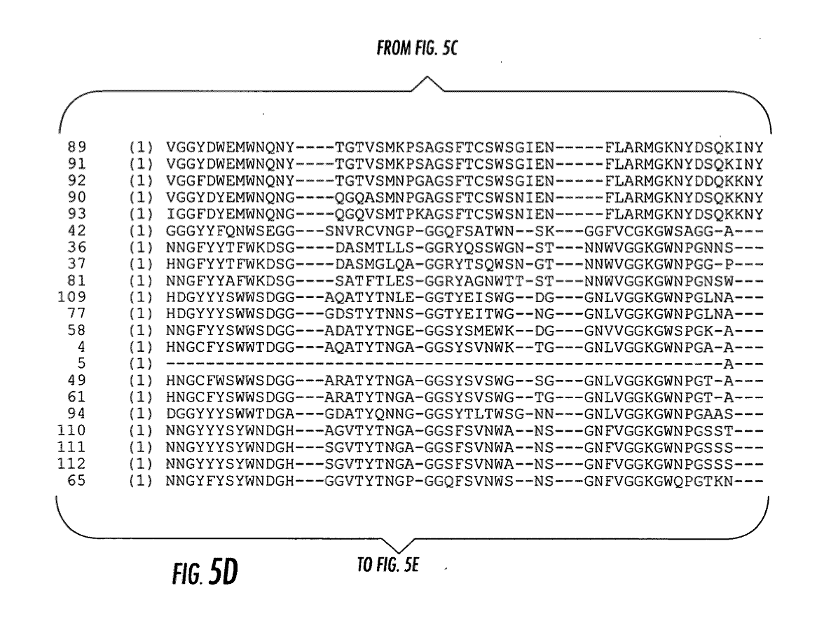

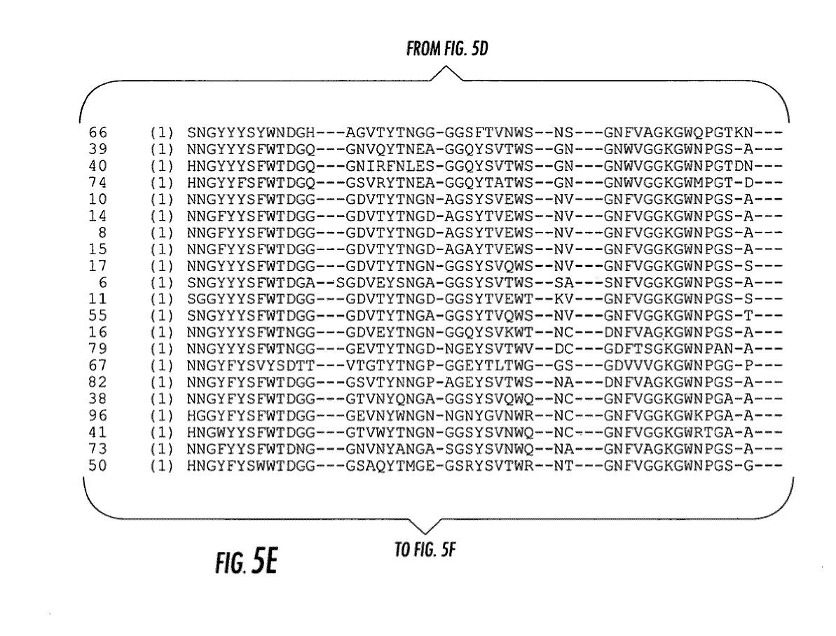

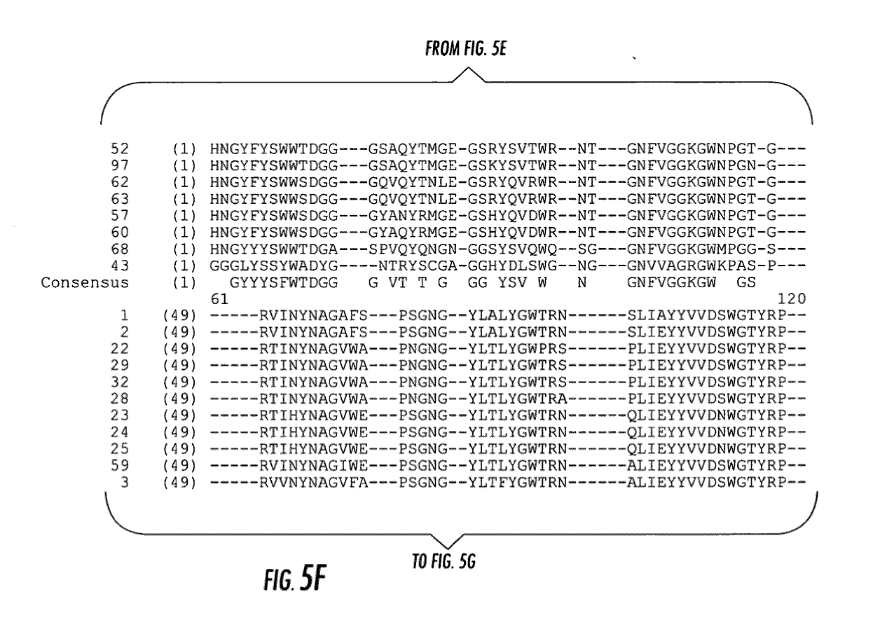

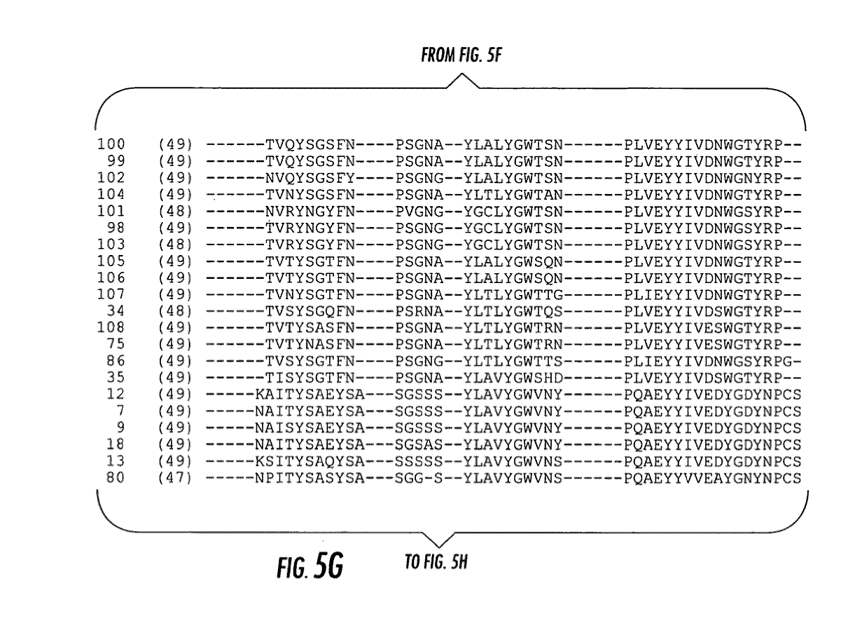

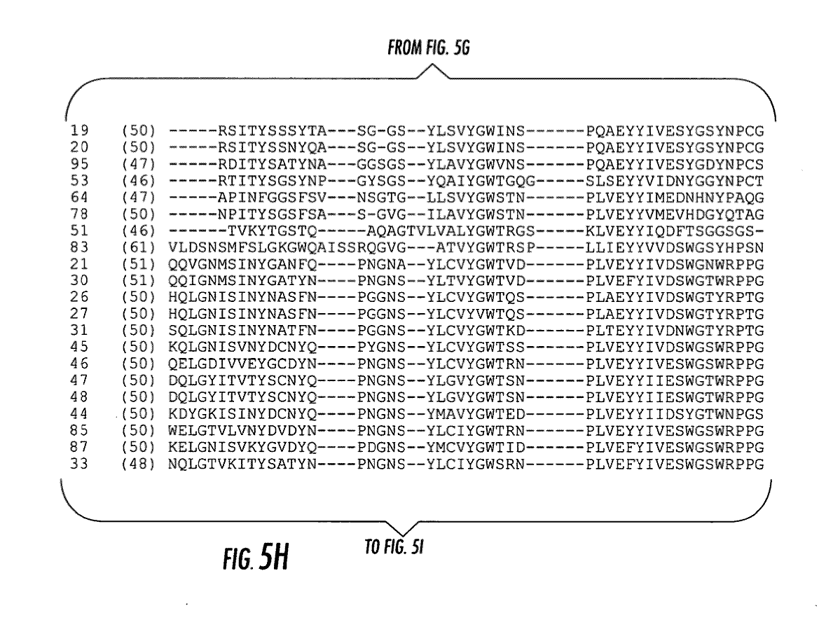

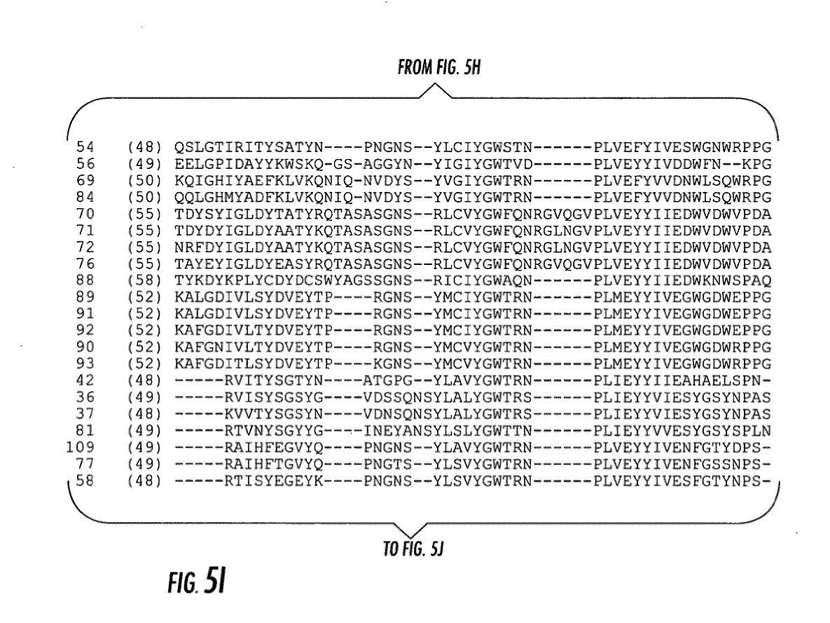

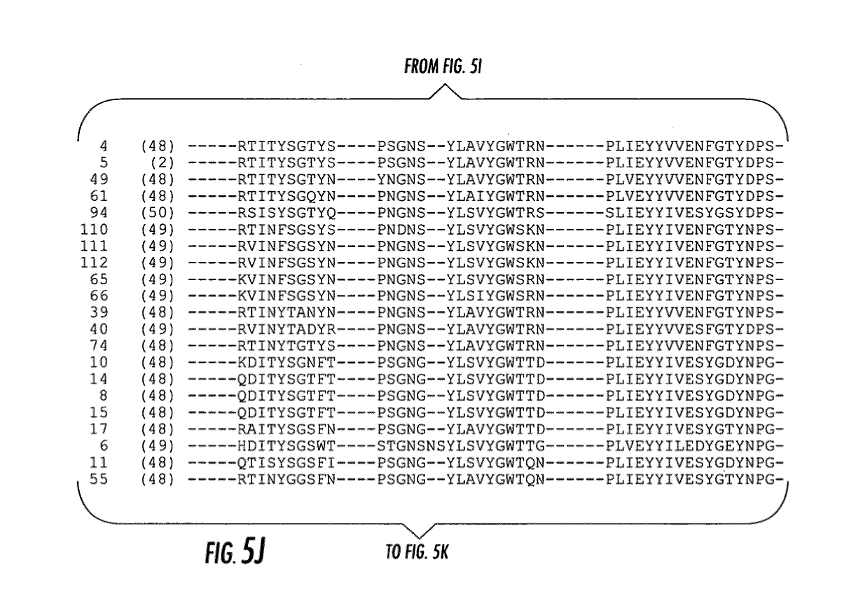

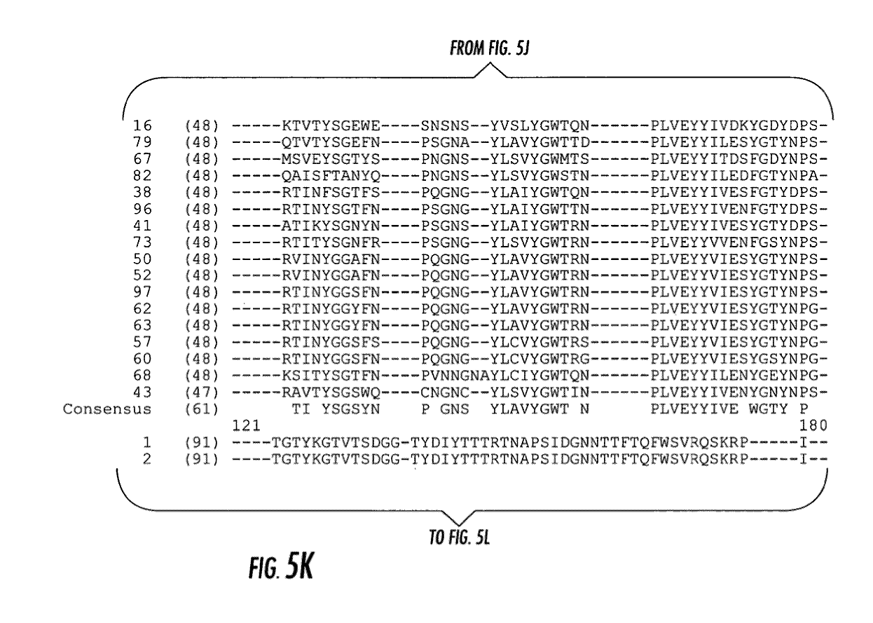

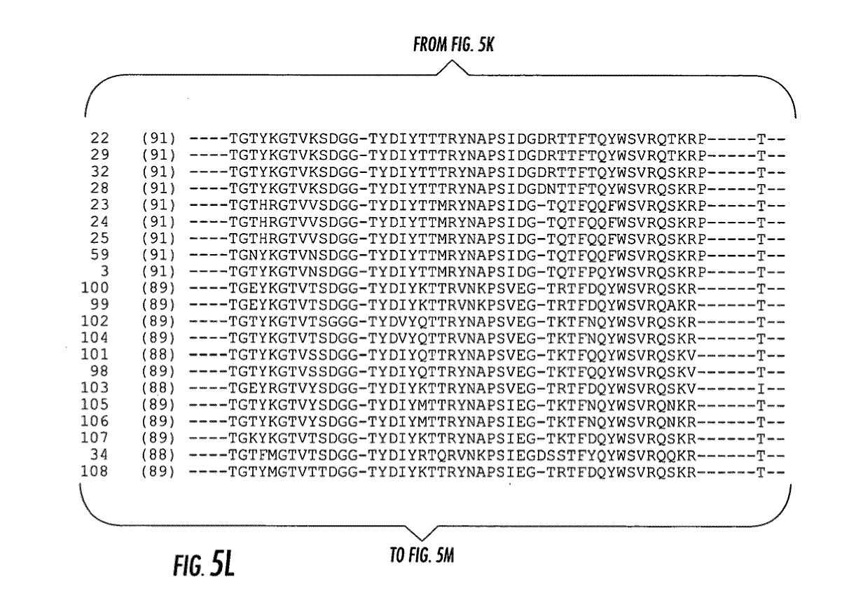

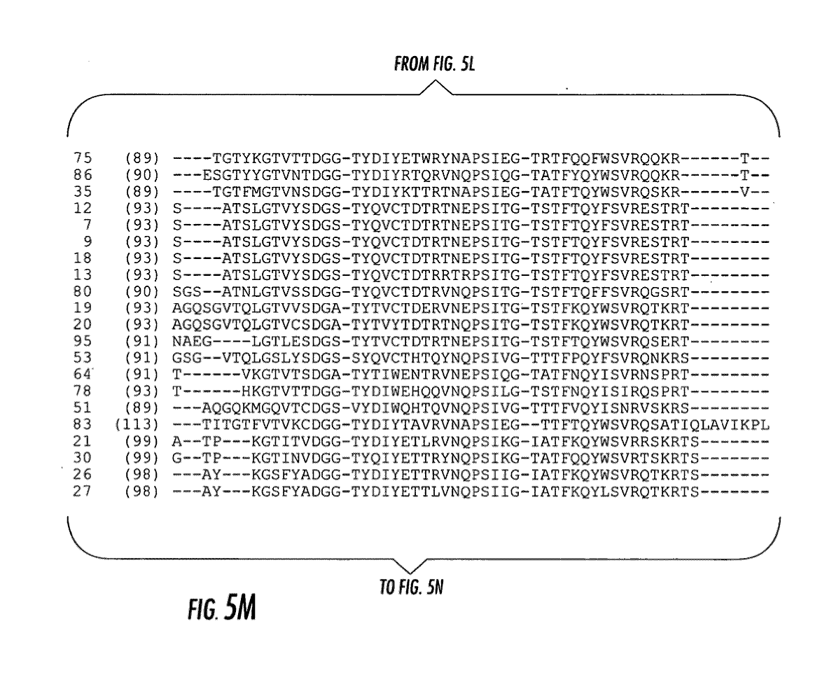

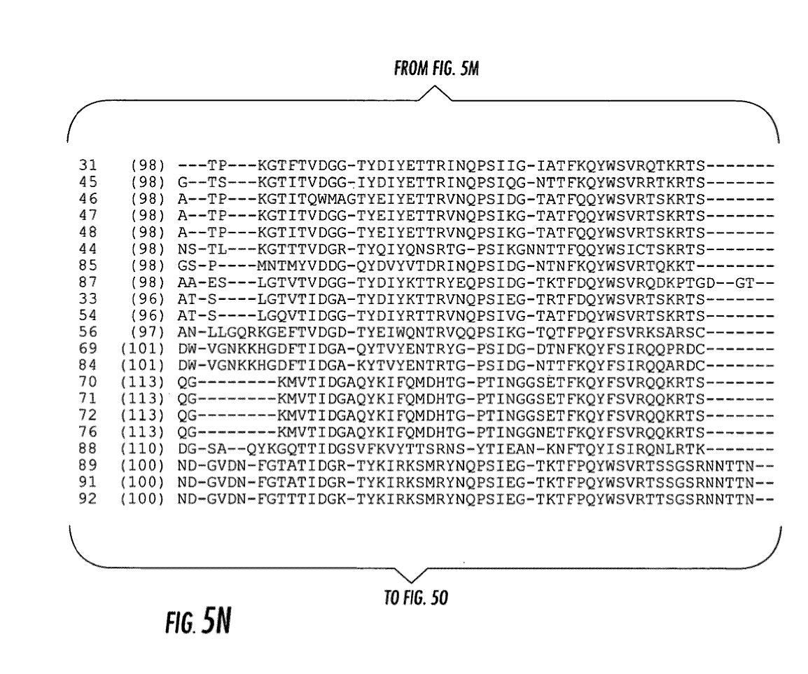

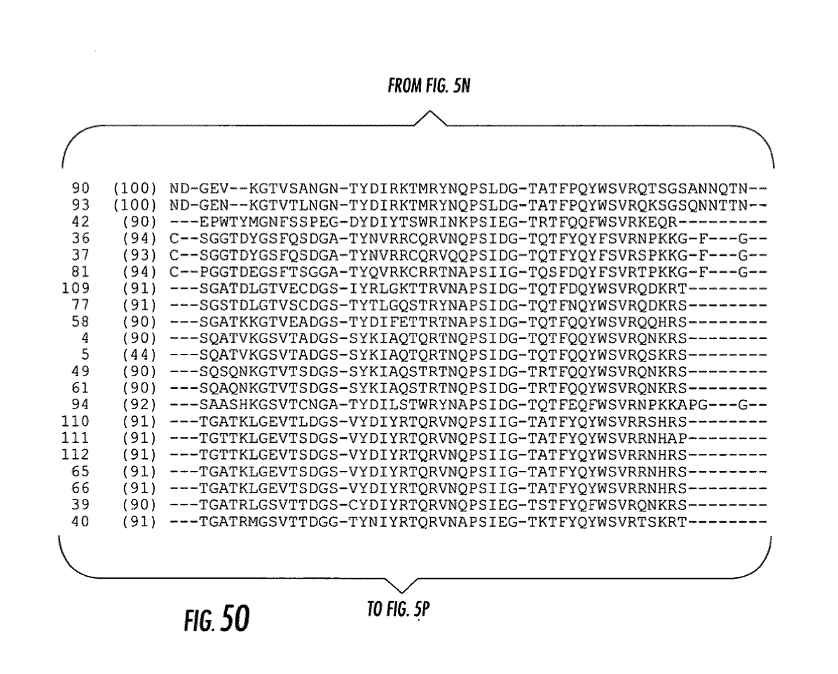

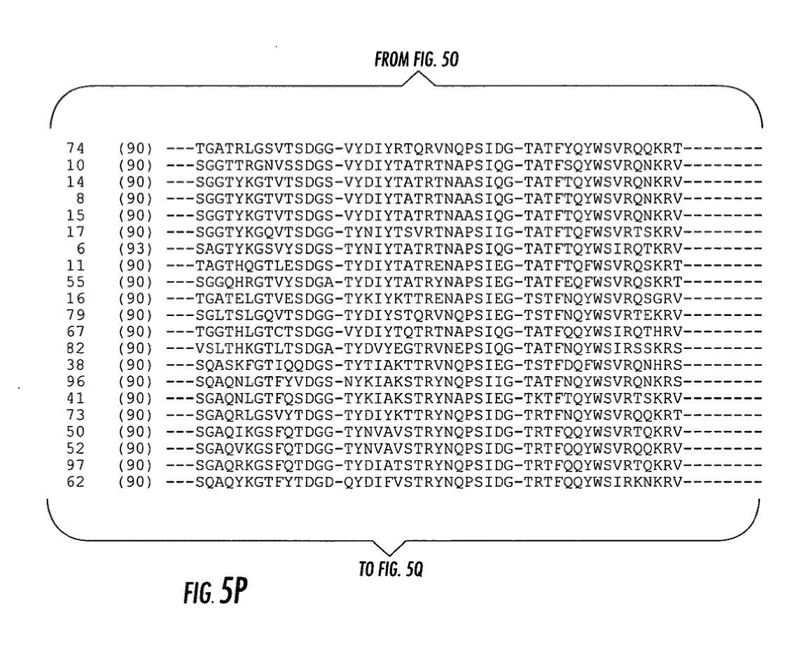

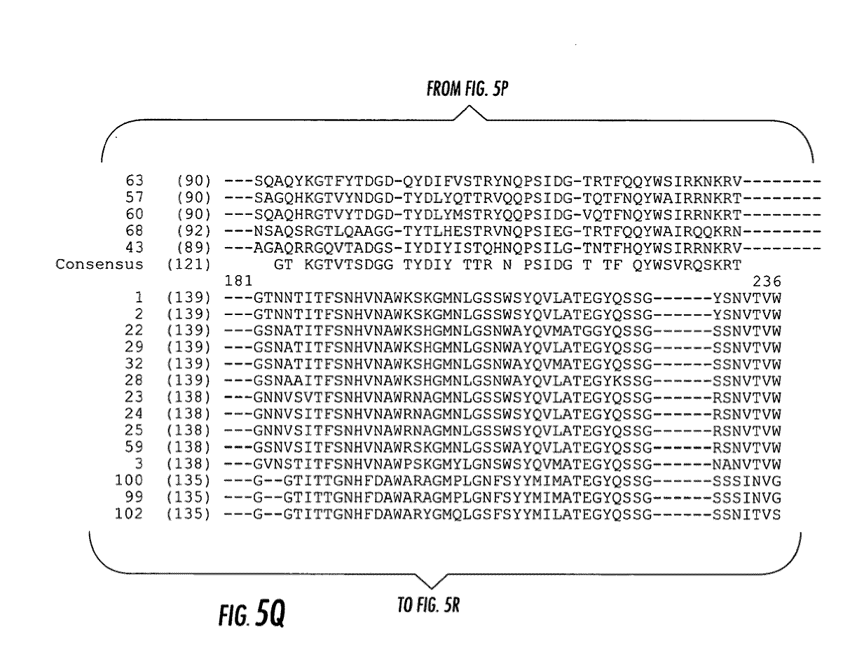

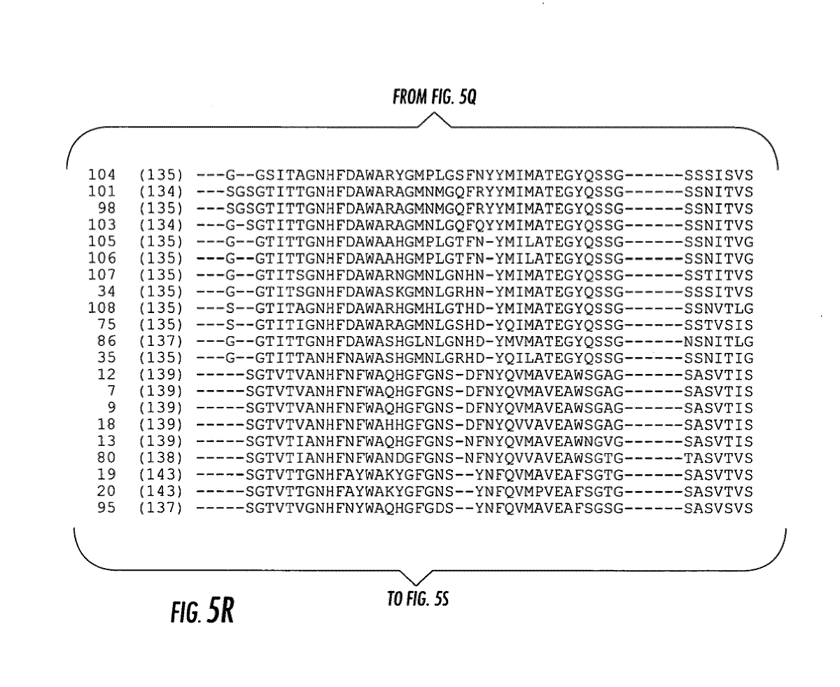

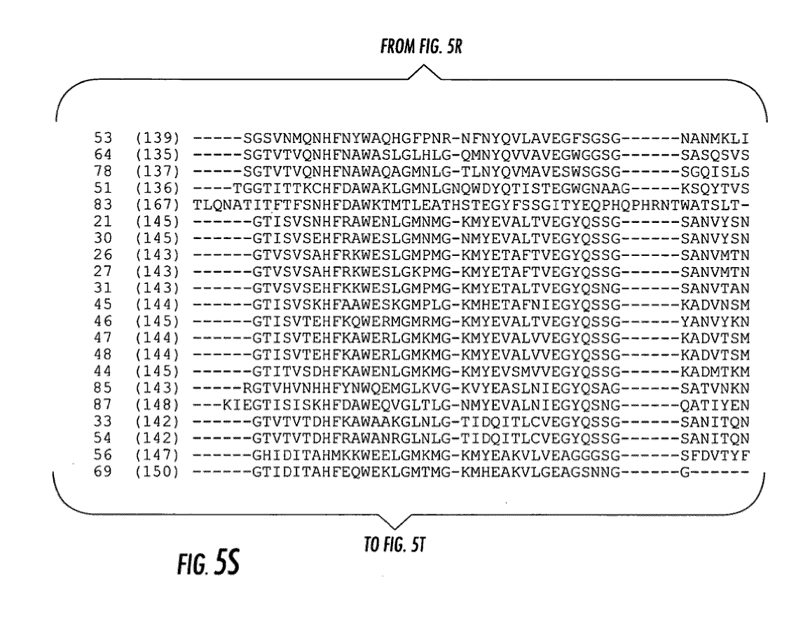

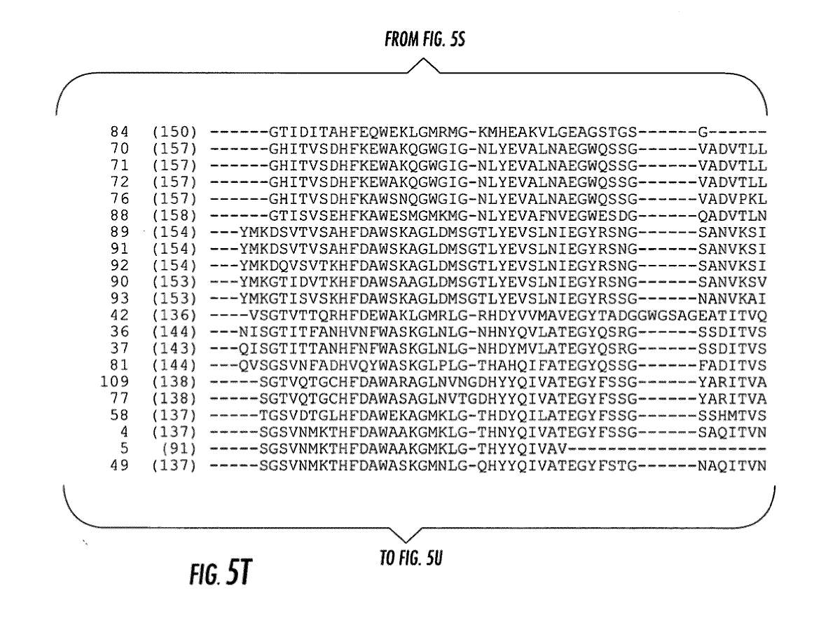

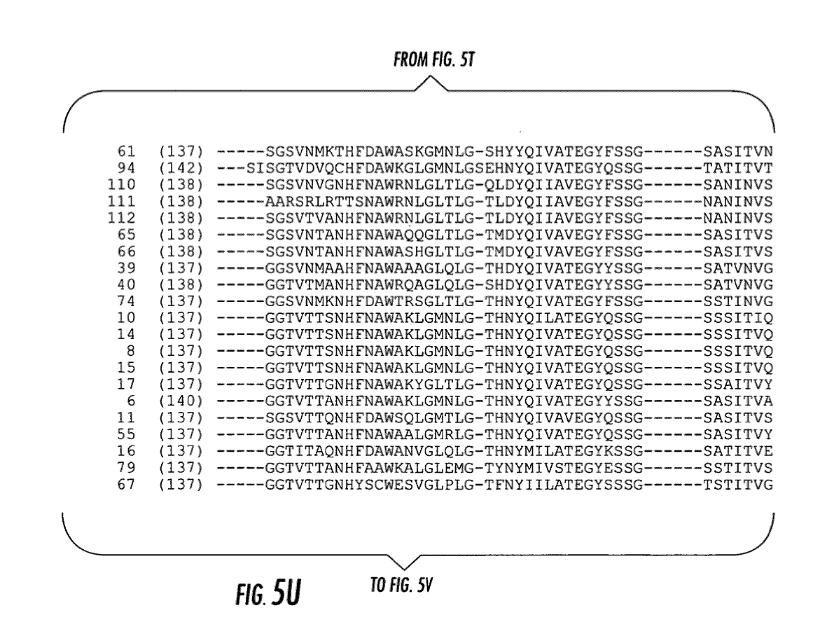

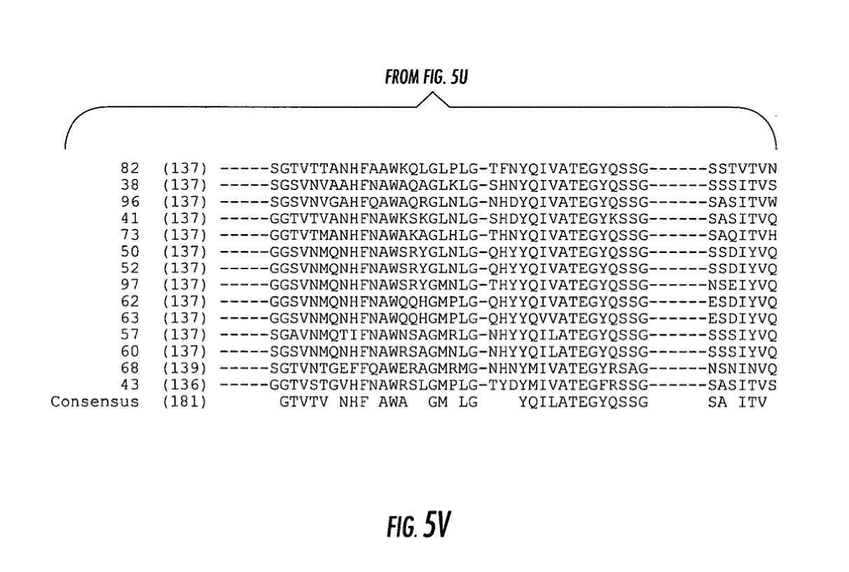

A xylanase, Xy1A1A, was identified by activity-based screening of a library made from an environmental sample. A gene encoding the wild-type Xy1A1A xylanase (SEQ ID NO. 3) was cloned into the bacterial expression vector pTrcHis. This vector was designated pTrcHis_Xy1A1A and is represented by FIG. 1 and SEQ ID NO. 114. To create this construct, the putative signal sequence of Xy1A1A was removed from the full length gene sequence resulting in a truncated xylanase gene. Furthermore, upon insertion of the truncated xylanase gene into pTrcHis, the open reading frame including the xylanase gene contained 5 additional codons at the 5′ end which were derived from the cloning vector and were not encoded by the xylanase gene. Sequence alignment between a translation of the full-length Xy1A1A coding sequence and other glycosyl hydrolase family 11 xylanases from the literature indicated that the glutamic acid at amino acid position 79 (amino acid numbering is based on the truncated Xy1A1A lacking the native signal sequence, with putative methionine at the N-terminal end of Xy1A1A counting as the amino acid #1) of Xy1A1A aligned with the conserved catalytic nucleophiles in these other proteins (see FIG. 5A-5V). Then, overlapping synthetic oligonucleotides (Primer 1 & 2) were designed to change the glutamic acid at position 79 to an alanine via site-directed mutagenesis using methods described by Statagene (Stratagene, La Jolla, Calif.).

SEQ ID NO. 118 (Xy1A1A aa)
73 T R N S L I E Y Y V V D S W 86
SEQ ID NO. 119 (Xy1A1A nuc):
GG ACG AGA AAT TCA CTC ATA GAA TAT TAC GTC GTT GAT AGC TGG
SEQ ID NO. 120 (Xy1A1A E79A aa):
73 T R N S L I A Y Y V V D S W 86
SEQ ID NO. 121 (Primer 1):
5′-GG ACG AGA AAT TCA CTC ATA GCT TAT TAC GTC GTT GAT AGC TGG-3′
SEQ ID NO. 122: (Primer 2):
5′-CCA GCT ATC AAC GAC GTA ATA AGC TAT GAG TGA ATT TCT CGT CC-3′

The vector pTrcHis_Xy1A1A was used as the template for the site directed mutagenesis procedure. Hotstart Turbo™Pfu DNA polymerase (Stratagene, LaJolla, Calif.) was used to amplify the modified plasmid from the parent molecule using the thermocycler settings below:

TABLE I

| Step | Temp (C.) | Time | Cycles |
|---|---|---|---|
| 1 | 94 | 30 seconds | 1 |
| 2 | 94 | 30 seconds | 16 |
| 3 | 55 | 30 seconds | |
| 4 | 68 | 18 minutes | |

| | | |
|---|---|---|
| 5 | 4 | 5 min-16 hour |

The site directed mutagenesis PCR resulted in the modification of the gene sequence from GAA (Glutamic Acid) to GCT (Alanine) This produced a protein that lacked the active site nucleophile necessary to perform catalysis. Amino acids other than alanine could also be placed into this location to produce the same effect (i.e., loss of catalytic activity) as described in the literature [references from Milan & others]. The resulting vector was named pTrcHis_Xy1A1A_E79A and is represented by FIG. 2 and SEQ ID NO. 115.

### EXAMPLE 2

### Production of Catalytically-Inactive Xylanase Protein in a Bacterial Expression Host

The pTrcHis_Xy1A1A_E79A vector was transformed into BL21 Star (pLysS) cells and plated on Luria broth agar plates containing 100 μg/mL ampicillin (LBₐₘₚ₁₀₀) by standard techniques [Sambrook et al]. Individual colonies were selected and inoculated into 3.5 mL of Terrific broth containing 50 μg/mL ampicillin and 25 μg/mL chloramphenicol (TB_{amp50-Chlor25}) and grown overnight at 37° C. with constant agitation. After overnight incubation, a portion of the culture was removed and a glycerol cryogenic stock was made from the culture for storage at −80° C.

From this glycerol stock, a sterile loop was used to inoculate a 20 mL of TB_{amp50-Chlor25} in a 250 mL flask. The culture was grown overnight at 37° C. with shaking at 200-250 rpm. On the following day, 5 milliliters of overnight culture was diluted into 1.5 liters of TB_{amp50-Chlor25}. This culture was incubated at 37° C. with shaking until the OD600 reached 0.6-1.0. Then, 7.5 mL of 200 mM isopropylthiogalactoside (IPTG) was added and the culture was incubated overnight at 16° C. with shaking at 200-250 rpm. The cells were subsequently harvested by centrifugation (10 minutes at 10,000×g, 4° C.). The cell pellet was frozen at −80° C. and then thawed to room temperature. The cell pellet was resuspended in 50 mM potassium phosphate buffer pH 7.0. The cells were disrupted by sonication and the cell debris was removed by centrifugation (30 minutes at 20,000 rpm, 4° C.). The supernatant was collected and dialyzed against 50 mM potassium phosphate buffer pH7.0 with 3.5 kDa cutoff membranes. The dialyzed supernatant was lyophilised and stored at 4° C. The lyophilizate was resuspended in water prior to use.

### EXAMPLE 3

### Preparation of Expression Constructs for the Production of Xy1A1A_E79A in the Yeast Host, Pichia pastoris

Construction of pCR4Blunt_Xy1A1A E79A

The BD6002E79A gene was amplified from pTrcHis2_BD6002E79 by PCR using synthetic oligonucleotides, primers 3 and 4, and Pfu DNA polymerase (Stratagene, LaJolla, Calif.) with thermocycler set to the parameters below:

Primer 3
(SEQ ID NO. 123)
5′-TTTCCCTCTCGAGAAAAGAGCTTCGACAGACTACTGGCAAAATTGG
Primer 4
(SEQ ID NO. 124)
5′-TTTTCCTTTTGCGGCCGCCTATTACCAGACCGTTACGTTAGAGTAC

| Step | Temp (C.) | Time | Cycles |
|---|---|---|---|
| 1 | 94 | 5 minutes | 1 |
| 2 | 94 | 30 seconds | 25 |
| 3 | 65 | 30 seconds | |
| 4 | 72 | 30 seconds | |
| 5 | 72 | 10 minutes | 1 |

| | | |
|---|---|---|
| 6 | 4 | Forever |

Primer 3 was designed to anneal at the codon that corresponds to amino acid 5 of the pTrcHis open reading frame containing the xylanase. In addition, the primer 3 added an XhoI restriction site and the Kex2 protease cleavage signal (Leu-Glu-Lys-Arg) in front of the mature xylanase coding sequence. Primer 4 included a double-stop codon after the xylanase gene. The BD6002E79A PCR product was subcloned into an intermediate pCR4-Blunt TOPO vector (Invitrogen, Carlsbad, Calif.). No mutations to the BD6002E79A gene were introduced during PCR amplification or cloning. The plasmid is designated “pCR4Blunt_Xy1A1A_E79A” and is represented by FIG. 3 and SEQ ID NO. 116.

Construction of pPIC9_Xy1A1A_E79A

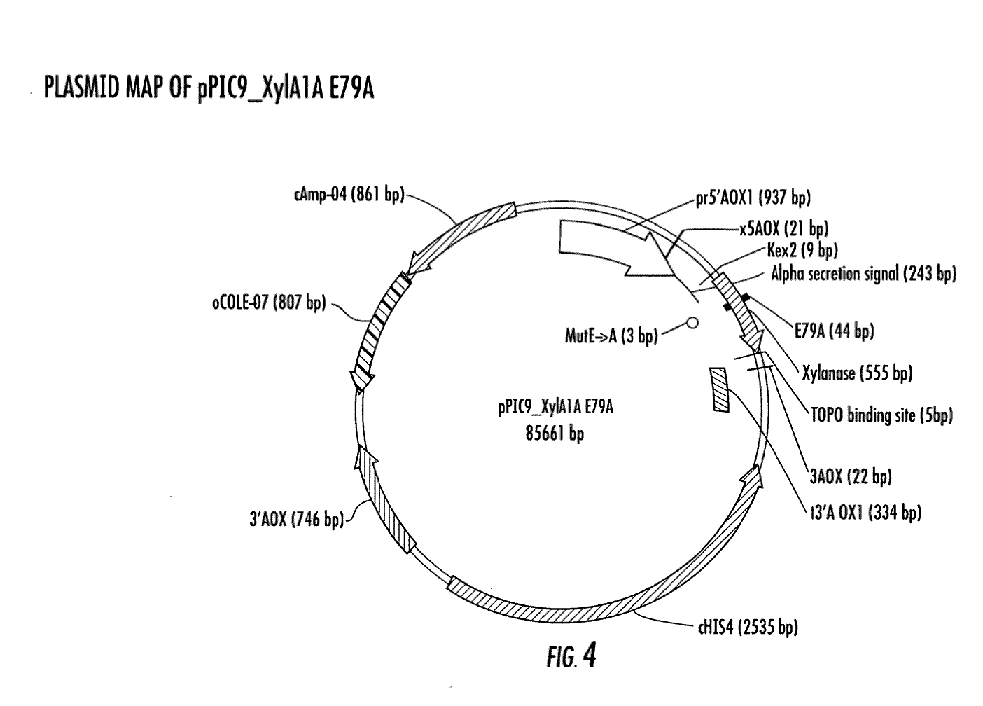

The intermediate vector harboring the PCR product, pCR4Blunt_Xy1A1A_E79A, was digested to completion with XhoI and EcoRI (New England Biolabs) and the approximately 0.5 kb fragment corresponding to the Xy1A1A_E79A gene was purified by methods described by Qiagen (Qiagen, Valencia, Calif.). In a parallel reaction, the yeast secretory expression vector pPIC9 (Invitrogen, Carlsbad, Calif.) was digested to completion with XhoI and EcoRI. The digestion mixture was electrophoresed through a 0.8% TAE gel and the 8.0 kb vector purified by methods described by Qiagen (Qiagen, Valencia, Calif.). The gel purified insert and vector components were ligated using T4-ligase (New England Biolabs, Beverly, Mass.). The ligation reaction was transformed into chemically competent E. coli TOP10 cells) and spread onto agar plates containing LB_{Amp100}. This cloning strategy produces a fusion protein in which the Saccharomyces cerevisiae α-mating factor pre-pro-peptide secretion signal is fused in frame to the N-terminus of the Xy1A1A_E79A gene. The fusion peptide is secreted from the cell after production. During the secretion process, the α-factor peptide portion of the fusion protein is cleaved by the Kex2 protease and Xy1A1A_E79A protein is released into the extracellular environment. Other signal peptides could be utilized by one skilled in the art. The Xy1A1A_E79A gene in this construct is under the control of the P. pastoris alcohol oxidase-1 (AOX1) promoter that is inducible with methanol. Other promoters could be utilized by one skilled in the art. DNA was purified from colonies grown on the selective media by methods described by Qiagen (Qiagen, Valencia, Calif.). The gene sequence was confirmed using plasmid specific 5AOX and 3AOX sequencing primers supplied by the manufacturer (Invitrogen, Carlsbad, Calif.). After sequence confirmation, the pPIC9_Xy1A1A_E79A plasmid, represented by FIG. 4 and SEQ ID NO. 117, was retransformed into chemically competent E. coli TOP10 cells as previously described and a glycerol stock was prepared using known methods.

### EXAMPLE 4

### Creation of a Pichia pastoris strain Producing Xy1A1A_E79A Preparation of pPIC9_Xy1A1A_E79A DNA for transformation of P. pastoris

A 50 mL culture of TB broth supplemented with 100 μg/mL ampicillin was inoculated with the glycerol stock of E. coli TOP10 cells harboring pPIC9_Xy1AA_{—}1E79A, and grown over-night at 37° C. DNA was purified from the culture by methods described by Qiagen (Qiaprep Midiprep protocol, Qiagen, Valencia, Calif.). The isolated plasmid DNA was digested over-night with Bg/II endonuclease (New England Biolabs, Beverly, Mass.). The digestion mix was electrophoresed through a 0.8% Tris Acetate EDTA (TAE) agarose gel and the 6.2 kb fragment corresponding to the Xy1A1A_E79A integration cassette purified from the gel by methods described by Qiagen (QiaQuick gel purification protocol, Valencia, Calif.). A portion of the purified fragment was electrophoresed through a 0.8% TAE gel to confirm complete digestion and its relative concentration. In addition, a portion of the purified fragment was transformed into chemically competent E. coli TOP10 cells to confirm that no residual circularized plasmid harboring the ampicillin marker contaminated in the sample. The entire transformation mix was spread on an LB_{Amp100} plate and incubated at 37° C. overnight. No colonies grew on the plate. Preparation of P. pastoris GS 115 cells for transformation

All microbiological manipulations were conducted in a laminar flow hood using aseptic techniques. Pichia pastoris GS115 yeast cells (Invitrogen, Carlsbad, Calif.) were prepared by streaking the cells onto YPD agarose plates. Following overnight growth at 30° C., a single yeast colony from the YPD agarose plate was transferred to 7 mL of YPD broth and grown at 30° C. overnight. A portion of this “seed culture” was used to inoculate a sterile 2-liter, baffeled flask containing 250 mL of YPD broth. This culture was grown with vigorous shaking overnight at 30° C. to an optical density OD600=1.5. The cells were harvested by centrifugation at 4000×g, 4° C., 5 minutes, and resuspended in 80 mL of sterile distilled deionised water. Ten milliliters of 10×TE buffer (10 mM Tris-HCl, 0.1 mM EDTA), pH 7.5 was added to the suspension followed by 10 mL of 1 M lithium acetate (LiAc). The cell suspension was incubated at 30° C. with gentle swirling. After 45 minutes of incubation, 2.5 mL of 1 M DTT was added and the cell suspension returned to incubate at 30° C. for an additional 15 minutes. The cells were then washed in a series of water washes and finally resuspended in 5 mL of ice-cold 1 M sorbitol.

Transformation of pPIC9_Xy1A1A_E79A DNA into Pichia pastoris GS115

Purified DNA (100 ng) of the Xy1A1A_E79A expression cassette from the Bg/II digested pPIC9_Xy1A1A_E79A plasmid was mixed with 80 μL of LiAc/sorbitol-treated Pichia pastoris GS115 cells in a 0.2 cm electroporation cuvette and incubated on ice for 5 minutes. The electroporation cuvette was placed into a BioRad Gene Pulser II instrument and pulsed using settings of 1.5 kV, 25 mF, and 200 W. Ice-cold sorbitol (0.5 mL) was added to the electroporation mix which was then plated onto histidine deficient, minimal media-dextrose (MD); 1% Yeast Extract, 2% Peptone, 100 mM KPO4 pH 6, 4×10⁻⁵ Biotin, 1% Glucose) agar plates. P. pastoris strain GS115 is a histidine auxotroph and is unable to grow in the absence of histidine, but stable transformants containing the his4 gene on the Xy1A1A_E79A expression cassette are restored to histidine prototrophy and are capable of growth on histidine-free media. Growth at 30° C. for 3 days produced a number of histidine prototrophic transformants. LiAc/Sorbitol washed GS115 cells electroporated in the absence of transforming DNA were plated onto MD and MD/histidine agar plates as controls. The GS115 cells with no transforming DNA present during electroporation generated no colonies capable of growth on MD plates lacking histidine.

### EXAMPLE 5

### Identification of P. pastoris transformants producing Xy1A1A_E79A Expression of Xy1A1A_E79A Protein in Pichia pastoris

From the primary transformants on MD plates, 24 single, isolated colonies were picked and replica plated onto an MD agar master plate. These colonies were subsequently also replica plated to histidine-deficient, minimal-media with 1.0% methanol (MM); 1% Yeast Extract, 2% Peptone, 100 mM KPO4 pH 6, 4×10⁻⁵ Biotin, 1% Methanol) agar plates containing 0.1% Azo-wheat arabinoxylan (Azo-WAXY). The MM Azo-WAXY plates were incubated at 30° C. for two days. No xylanase activity was observed for any of the transformants. Concurrently, three millilitres of BMGY (Buffered Glycerol Complex Medium; 1% Yeast Extract, 2% Peptone, 100 mM KPO4 pH 6, 4×10−5 Biotin, 1% Glycerol) liquid media was added to each well in a sterile 24-well culture block using a repeat pipettor. Each well was inoculated with a representative E79A isolate from the MD agar master plate. The block was covered with gas permeable tape and the culture block incubated at 30° C., 175 rpms. After two days of incubation, the block was removed from the shaker and centrifuged at 4000 rpm for 10 minutes to pellet the cells. The BMGY media was aspirated from the cells immediately after centrifugation by using a vacuum trap apparatus. Three millilitres (3 mL) of BMMY (Buffered Glycerol Complex Medium; 1% Yeast Extract, 2% Peptone, 100 mM KPO4 pH 6, 4×10⁻⁵ Biotin, 1% Methanol) liquid media was added to each well and the cells resuspended by gentle mixing. The block was covered with fresh gas permeable tape and the culture block incubated at 30° C., 175 rpms. The following morning, the block was removed from the 30° C. shaker and 300 μL of 10% methanol added to each well for a final concentration of 1% methanol (v/v) using a repeat pipettor. The block was covered with fresh gas permeable tape and returned to the shaker to incubate at 30° C., 175 rpm. This process was repeated for three days. On the final day, the block was removed from the 30° C. shaker and centrifuge at 4000 rpm for 10-15 minutes. The clarified supernatants were collected aseptically.

Preparation of Stabs Cultures and Glycerol Stocks for Long-Term Storage of P. pastoris Transformants

Glycerol freezer stocks were prepared by inoculating 5 mL of liquid MD media for isolates 53-12 and 53-20 from the MD master plate and grown at 30° C., overnight on a rotating culture wheel. Sterile glycerol (1 mL) was mixed into each culture to yield a 15% (v/v) mixture of glycerol to culture. Each culture was aliquoted into 4 sterile cryo-vials and stored at −80° C.

Characterization of Xy1A1A_E79A P. pastoris Expression Host Screening for MutS Phenotype

In order to identify the MutS phenotype, the 2 Xy1A1A_E79A-positive isolates were streaked onto histidine-deficient, minimal-media containing 1.0% methanol (MM) agar plates along side a MutS positive control (GS115 harboring pPIC9-secHSA; Invitrogen, Carlsbad, Calif.) and a Mut+ control (GS115 harboring pPIC3-β-Gal; Invitrogen, Carlsbad, Calif.). The plates were incubated at 30° C. for 4 days and the growth on MM recorded. Isolate 53-12 exhibited slow growth on MM media comparable to the MutS control.

### PCR Screen for Presence of Integration Casette

Genomic DNA from the 2 Xy1A1A_E79A-positive isolates, as well as GS115, were isolated from 2 mLs of the 7 mL YPD liquid cultures using the YeaStar Genomic DNA Purification kit (Zymo Research, Orange, Calif.). This DNA was used as a template in PCR reactions to screen for the MutS genotype. Synthetic oligonucleotide primers 5 and 6 were designed to amplify from the genomic sequence flanking the AOX1 promoter on the 5′ side to the 3′ end of the HIS4 gene. Synthetic oligonucleotide primers 7 and 8 were designed to amplify from 5′ end of the AOX1 transcription terminator to the genomic sequence flanking the AOX1 locus on the 3′ side. These two PCR products overlap by ˜400 bp in the middle and, together, span the entire AOX1 insertion site

Primer 5:

| | |
|---|---|
| 5′-GCTTCTTGCTGTAGAATTTGGGC | SEQ ID NO. 125 |
| Primer 6: | |
| 5′-CCAAAGCGGTCGGACAGTGCTCCG | SEQ ID NO. 126 |
| Primer 7: | |
| 5′-GGAATTCGCCTTAGACATGACTGTTCCTC | SEQ ID NO. 127 |
| Primer 8: | |
| 5′-GTTGGCCAGTAAATATAGAGATCAAGC | SEQ ID NO. 128 |

Genomic DNA was amplified with HotStarTaq™ polymerase mix (Qiagen, Valencia, Calif.). The thermocycler profile used in this experiment was the following:

TABLE 2

| Step | Temp (C.) | Time | Cycles |
|---|---|---|---|
| 1 | 94 | 15 minutes | 1 |
| 2 | 94 | 30 seconds | 35 |
| 3 | 55 | 30 seconds | |
| 4 | 72 | 7 minutes | |
| 5 | 72 | 10 minutes | 1 |

| | | |
|---|---|---|
| 6 | 4 | Forever |

Isolate 53-12 resulted in the amplification of the predicted 3.0 and 4.5 kb fragments with primers 3 and 4 and primers 5 and 6, respectively. In addition, GS115 produced no product with primers 3 and 4 and produced the predicted 1.5 kb fragment with primers 5 and 6. The experiment indicated that in P. pastoris Xy1A1A_E79A expression isolate 53-12, the native AOX1 gene sequence was deleted and had been replaced with the Xy1A1A_E79A expression cassette through the process of double homologous recombination. Molecular replacement of the native AOX1 gene with the Xy1A1A_E79A expression cassette alters Pichia's ability to metabolize methanol resulting in the MutS phenotype. Recombination at another homolgous region, such as the his4 or 3AOX-TT loci, leaves the native AOX1 gene unaltered, and Pichia displays a normal growth rate in media containing methanol (Mut⁺). Isolate 53-12, was chosen for further DNA characterization.

### Hybridization Screen of the Xy1A1A_E79A Expression Cassette

In support of PCR experimental results that demonstrated replacement of the AOX1 gene in the Pichia genome with the Xy1A1A_E79A expression cassette and that showed the absence of the ampicillin resistance gene, a series of hybridization experiments were conducted. Two micrograms of isolate 5312 genomic DNA was digested using BamHI, Bg/II, EcoRI, HindIII, XhoI, & NotI. The digests were run through a 0.8% TAE agarose gel and transferred on to a nitrocellulose membrane utilizing standard Southern blotting protocols. DNA hybridization probes specific for the Xy1A1A_E79A gene (xyn) and the vector backbone (backbone), which contains the ampicillin resistance gene and pUC origin of replication, were prepared. The xyn and backbone probes were generated by polymerase chain reaction using gene specific primers. The products were gel purified and radiolabelled with 5′-[a-32P]-dCTP using the Rediprime II random prime labeling system (Amersham Biosciences, Piscataway, N.J.). Following hybridization with the backbone probe in PerfectHyb™ Plus Hybridization Buffer (Sigma-Aldrich, St. Louis, Mo.) at 65° C., the blot did not show any hybridizing bands, with the exception of the positive control which produced a band of approximately 2.3 kb. This experiment indicated that the ampicillin gene, the pUC origin of replication and any extraneous vector sequence did not integrate in the transgenic isolate 5312. A similar blot was probed with xyn probe by methods previously described. The blot produced a band equal to 6.2 kb using the Bg/II restriction enzyme, confirming that the transgenic P. pastoris Xy1A1A_E79A expression isolate 5312 contained an intact, single copy of Xy1A1A_E79A integration cassette. All other restriction digests produced hybridizations of the xyn probe of expected size. In summary, all characterizations of P. pastoris Xy1A1A_E79A expression isolate 5312 by PCR, Southern blotting, and by growth characteristics on methanol containing media demonstrates that this isolate had a His+, MutS genotype and that it contained a single copy of the Xy1A1A_E79A expression cassette inserted into the AOX1 gene and do not contain an ampicillin resistance gene.

Preparation of the Xy1A1A_E79A P. pastoris Master Cell Bank

From the MD glycerol freezer stock of isolate 53-12, a master cell bank was made; hereafter named P. pastoris isolate 53-12. The clone was streaked onto a MD plate and incubated at 30° C. until the appearance of colonies. A single colony was picked from the MD plate and inoculated into 7 mL of YPD and incubated at 30° C. for 12-16 hours. A 2.8 L baffled flask containing 250 mL of YPD medium was inoculated with the entire contents of the overnight starter culture. The culture was grown at 30° C. on a shaker at 150 rpm for 6-8 hours. Sterile glycerol (110 mL) was added when the OD600 reached 2.0-3.0 and 1.0 mL aliquots of the cells were distributed into 81 sterile screw-capped cryogenic vials (Nalgene, Rochester, N.Y.). The cryogenic vials were kept at room temperature for 5 minutes and stored a freezer at −80° C. for long-term storage.

Purity of the P. pastoris Xy1A1A_E79A Master Cell Bank

A sample from one of the vials in the master cell bank was resuspended rich media then plated onto YPD agar plates and incubated overnight to generate numerous individual colonies on the plate (˜100). These were examined visually and were found to have a homogenous colony morphology that was identical to that of the parent strain P. pastoris GS 115. Numerous colonies from the YPD plate were transferred to MD and MM agar plates. All colonies were able to grow on both MD and MM agar that lack histidine, indicating that like isolate 53-12, but unlike the parent strain GS115, they all had a His⁺ phenotype. Furthermore, all colonies grew slowly on MM agar containing methanol as a source of carbon, indicating that like isolate 53-12, but unlike strain GS115, they had a Mut^{S} phenotype that is expected of AOX1 mutants. The results of these analyses indicate that the MCB described herein is pure and uncontaminated with other micbrobes.

Genetic Stability of P. pastoris Xy1A1A_E79A Clone

The genetic stability of the Xy1A1A_E79A expression cassette in isolate 53-12 was tested by conducting 20 consecutive plating experiments on MD agar. Cells from one of the MCB cryogenic vials were transferred onto a MD agar plate and grown up for 36-48 hours at 30° C. (plate 1). From plate 1, a single colony was picked and replated onto a second MD plate. This cycle of single colony picking and replating was conduced 20 consecutive times. Genomic DNA was purified from YPD liquid culture inoculated with a single colony from plates 1 and 20. This DNA was used for Southern hybridizations as described previously. The hybridizing fragments for genomic DNA prepared from plates 1 and 20 were of identical size indicating that the insertion of the Xy1A1A_E79A cassette was stable. From the 20 restreaked plates, liquid cultures were established with colonies from plates 1 and 20 for protein expression analysis. A single colony from each of these plates was used to inoculate 100 mLs of BMGY media. Cells were grown up overnight at 30° C., spun down and resuspended in 10 mLs of BMMY. Cultures were incubated at 30° C. for 96 hours with the addition of MeOH every day to a final concentration of 0.5% (v/v). At the end of the fermentation period, clarified supernatant broth was analyzed by anti-xylanase ELISA. Clones from both plates produced similar amounts of Xy1A1A_E79A. Molecular characterization of DNA integrity and protein expression from cells from plates 1 and 20, demonstrate the stability of the integrated Xy1A1A_E79A expression cassette in the genome of Pichia pastoris GS115 and expression of the Xy1A1A_E79A gene within it.

### Identification of Xy1A1A_E79A Expressing Transformants by ELISA

Clarified supernatants from methanol-induced P. pastoris Xy1A1A_E79A transformants were diluted in ELISA diluent (1.17 g/L Na₂HPO₄, 0.244 g/L NaH₂PO₄.H₂O, 8.18 g/L NaCl, 10 g/L BSA, 0.5 mL/L Tween20, 0.2 g/L NaN₃, pH 7.4) and analysed by a quantitative sandwich assay that employs two polyclonal antibodies. Rabbit and goat anti-xylanase Xy1A1B antibodies were immunoaffinity purified (IAP) using immobilized xylanase (Xy1A1B). First, one hundred microliters of goat anti-xylanase IAP antibodies at 1 μg/ml in borate-buffered saline (BBS; 6.19 g/L boric acid, 9.50 g/L Na₂B₄O₇.10H₂O, 4.39 g/L NaCl, pH 8.5) was added to a Nunc Maxisorp C96 plate and incubated overnight at 4° C. The plate was washed 3 times with ELISA wash buffer (1.21 g/L Tris (Trizma), 0.5 mL/L Tween 20, 0.2 g/L NaN3, pH 8.0) and blocked with 300 microliters of ELISA diluent for 45 minutes at room temperature. Then, the plate was washed 3 times with ELISA wash buffer. Next, 100 microliters of diluted culture supernatants were added and incubated 1.5 hours at room temperature. The plate was washed 5 times with ELISA wash buffer and 100 microliters of rabbit anti- xylanase IAP antibodies at 1 μg/ml in ELISA diluent was added to each well and incubated at 37° C. for 1 hour. The plate was washed 5 times with ELISA wash buffer and 100 μl of alkaline phosphatase-conjugated donkey anti-rabbit at 1 μg/ml in ELISA diluent was added to each well and incubated at 37° C. for 1 hr. The plate was washed 5 times with ELISA wash buffer and 100 microliters of alkaline phosphatase substrate solution (p-nitrophenyl phosphate) was added to each well and incubated for 30 minutes at room temperature. The absorbance at 405 nm was measured with a reference filter at 492 nm. Of the 24 isolates, 12 were positive for the presence of a xylanase-like protein.

### Identification of Xy1A1A_E79A Expressing Transformants by Recovery of Xylanase Activity

Clarified supernatants from methanol-induced P. pastoris Xy1A1A_E79A transformants were diluted 1:5 in 50 mM McIlvaine buffer pH 5.4. Five hundred milligrams of wheat flour was dispensed into each well of a 24 well plate. The diluted Xy1A1A_E79A supernatants were transferred to the wells containing the wheat flour samples. Then, diluted xylanase Xy1A1A was added to all wells and stir bars were added to each well and the contents were mixed for 20 minutes at room temperature. The solids were removed by centrifugation (10 minutes at 1,000×g, r.t.). The supernatants were removed and assayed using azo-WAXY as substrate. For this assay, an azo-WAXY substrate (1.0 g) was added to 90 milliliters of boiling water and stirred for 10 minutes. The solution was cooled and adjusted to 100 mL with water. The substrate was dispensed into a 24 well plate (500 μL/well) and a stir bar was added to each well. The plate containing substrate and the plate containing clarified P. pastoris supernatant, wheat extract, and xylanase were equilibrated to 37° C. for at least 5 minutes. Then, the reaction was initiated by adding 500 μL of sample to substrate. The plate was incubated at 37° C. for 10 minutes with occasional mixing. Then, 2.5 mL of 95% ethanol was added to each well and the plate was gently shaken to mix. After ten minutes at room temperature, the plate was centrifuged for 10 minutes at 1,000×g and room temperature. The 4×200 μL of supernatants were drawn from each well and placed in 4 wells of a 96 well plate. The absorbance at 595 nm was measured in a plate reader. Wells containing Xy1A1A_E79A were identified as those having blue color. This indicated that the Xy1A1A_E79A protein was blocking the action of xylanase inhibitors and allowing the added xylanase Xy1A1A to degrade the arabinoxylan substrate. Of the 24 isolates tested, 9 showed recovery of xylanase activity. Of the 9 isolates that were positive for recovery of xylanase activity, all of them were positive for the presence of a xylanase-like protein by the ELISA.

### EXAMPLE 6

### Determination of reduced Xylanase Activity

The xylanase activity of E. coli- and P. pastoris-produced Xy1A1A_E79A was measured and compared to the xylanase activity of E. coli- and P. pastoris- produced Xy1A1A. Samples of lyophilized Xy1A1A_E79A proteins were resuspended to 1 mg/mL of solid in 100 mM sodium acetate buffer pH5.3. The Xy1A1A_E79A proteins were assayed without further dilution. Samples of lyophilized Xy1A1A proteins were resuspended in 100 mM sodium acetate buffer pH5.3 and diluted ˜1:10000.

Protein concentration for E. coli- and P. pastoris-produced Xy1A1A_E79A and the E. coli- and P. pastoris-produced Xy1A1A were determined using the Bicinchoninic acid (BCA™) method (Pierce, Rockford, Ill.) in a microtiter plate format and used to calculate the amount of protein per assay.

Enzymatic activity was determined using wheat arabinoxylan as substrate and measuring the release of reducing ends by reaction of the reducing ends with 3,5-dinitrosalicylic acid (DNS). The substrate was prepared as a 1.4% w/w solution of wheat arabinoxylan (Megazyme P-WAXYM) in 100 mM sodium acetate buffer pH5.3. The DNS reagent consisted of 0.5% w/w, 15% sodium potassium tartrate, and 1.6% w/w sodium hydroxide. To perform the assay, five hundred microliters of substrate were combined with 200 microliters of each sample. After incubation at the desired temperature for the desired length of time (15 minutes for Xy1A1A and Xy1A1A_E79A proteins), 700 microliters of DNS reagent was added. The contents were mixed and placed at 100° C. for 10 minutes. The contents were allowed to cool and then transferred to cuvettes and the absorbance at 540 nm was measured relative to known concentrations of xylose. The choice of enzyme dilution, incubation time, and incubation temperature could be varied by a person of ordinary skill in the art.

The activity of the E. coli-produced Xy1A1A was 635 U/mg of solid and the activity of the Pichia pastoris-produced Xy1A1A was 4439 U/mg of solid. The activity of E. coli- and P. pastoris-produced Xy1A1A_E79A proteins were below the assays limit of detection which represents 0.001 U/mg of solid or 0.0002% and 0.00002% of the activity observed for the E. coli- and P. pastoris-produced Xy1A1A proteins.

TABLE 3

| | Assay | [Total | | Calculated |
|---|---|---|---|---|
| | fold | Protein] | Abs @ | activity |
| Protein | dilution | assay | 540 nm | (umol/min/mg) |
| E. coli XylA1A | 2640 | 39 μg | 0.741 | 635 |
| P. pastoris XylA1A | 10681 | 1.73 μg | 1.160 | 4439 |
| E. coli XylA1A_E79A | 1121 | 24.07 μg | 0.166 | Below the limit |
| | | | | of detection |
| P. pastoris | 1019 | 0.520 μg | 0.175 | Below the limit |
| XylA1A_E79A | | | | of detection |

### EXAMPLE 7

### Identification of Xylanase Inhibitors in Wheat Kinetics of Inhibition by WXI

### Preparation of Wheat Extract for Purification of Xylanase Inhibitors

Soissons wheat flour was ground in a KTec kitchen mill to pass through a 1 mm screen (USA Standard Test Screen #18). Approximately fifty grams of flour was resuspended in 500 mL of 100 mM sodium acetate buffer pH5.3 with 0.02% w/v sodium azide (1×SAB) and stirred for 1 hour at room temperature. The slurry was centrifuged for 10 minutes at 5,000 rpm in a GS3 rotor at room temperature. The supernatant was collected and stored at 4° C. until used.

### Preparation of the Xylanase Affinity Column

Lyophilized xylanase, approximately 10 mg of Pichia pastoris produced Xy1A1A, was resuspended in 1.25 mL distilled water and brought up to 5 mL with 0.1M NaHCO3 pH8.3. This solution was dialyzed against 4 L of 0.1M NaHCO3 for 5.5 hr at 4° C. and then added to distilled water-washed affigel-10. The xylanase-coupled affigel-10 was poured into a 2 mL column.

### Purification of Xylanase Inhibitor

The xylanase affinity column was pre-eluted with 1 ml of 0.1M glycine-HCl pH2.5 followed by equilibration in PBS, pH7.3. Fifty mL of Soissons wheat extract was applied to the column by gravity. The column was then washed with PBS until no additional protein was eluted as monitored by absorbance at 280 nm. Proteins bound to the xylanase affinity column were eluted using 1 ml of 0.1M glycine-HCl pH2.5 followed by 6 ml of PBS. Two ml fractions were collected. (repeated 10 times) Absorbance at 280 nm was recorded for each fraction. The fractions containing protein based on A280 were combined (˜22 mL) and dialyzed extensively against 1×SAB with a 3 kDa cut-off membrane (Pierce Snake Skin) The dialyzed sample was labelled Wheat Xylanase Inhibitor (WXI).

### Preparation of Wheat Xylanase Inhibitor Solutions

The wheat xylanase inhibitor was diluted in 1×SAB in three decreasing concentrations: 16.2 μg/ml, 3.2 μg/ml, and 0.7 μg/ml. These three concentrations were labelled 1×SABWXIA, 1×SABWXIB, and 1×SABWIC, respectively. Protein concentration was determined using the Bicinchoninic acid method in a microtiter plate format and used to calculate the amount of protein per assay.

### Preparation of Xylanase Assay Samples

Three xylanases were used to determine the kinetics of inhibition by the wheat xylanase inhibitors. These xylanases were E. coli-produced Xy1A1A, Pichia pastoris-produced Xy1A1A, and E. coli-produced Xy1A1B. Each enzyme was diluted in 1×SAB, 1×SABWXIA, 1×SABWXIB, and 1×SABWXIC. The choice of enzyme dilution could be varied by one skilled in the art.

### Determination of the Xylanase Activity Inhibition

Enzymatic activity was determined using wheat arabinoxylan as substrate and measuring the release of reducing ends by reaction of the reducing ends with either DNS. Wheat arabinoxylan solutions were prepared at eight concentrations: 2.86%, 1.45%, 0.71%, 0.48%, 0.24%, 0.16%, 0.12%, and 0.09% final w/v in 1×SAB. The DNS reagent consisted of 0.5% w/w, 15% sodium potassium tartrate, and 1.6% w/w sodium hydroxide. To perform the assay, five hundred microliters of the substrate was combined with 200 microliters of each sample. After incubation at the desired temperature for the desired length of time, 700 microliters of DNS reagent was added. The contents were mixed and placed at 100° C. for 10 minutes. The contents were allowed to cool and then transferred to cuvettes and the absorbance at 540 nm was measured relative to known concentrations of xylose.

TABLE 4
Residual Activity of XylA1A, produced in E. Coli,
in the presence of wheat xylanase inhibitor

| | Buffer: | | | |
|---|---|---|---|---|
| % | 1xSAB | 1xSABWXIA | 1xSABWXIB | 1xSABWXIC |
| Waxy | % Residual Activity | | | |
| 2.86% | 100.0 | 19.6 | 38.8 | 69.2 |
| 1.45% | 100.0 | 18.1 | 36.0 | 69.6 |
| 0.71% | 100.0 | 23.1 | 36.9 | 69.7 |
| 0.48% | 100.0 | 28.2 | 35.8 | 76.1 |
| 0.24% | 100.0 | 31.2 | 39.4 | 107.9 |
| 0.16% | 100.0 | 1.1 | 27.3 | 74.1 |
| 0.12% | 100.0 | 0.6 | 22.1 | 64.7 |
| 0.09% | 100.0 | 0.2 | 41.2 | 106.2 |

TABLE 5
Residual Activity of XylA1B, produced in E. Coli, in the presence
of wheat xylanase inhibitor

| | Buffer: | | | |
|---|---|---|---|---|
| % | 1xSAB | 1xSABWXIA | 1xSABWXIB | 1xSABWXIC |
| Waxy | % Residual Activity | | | |
| 2.86% | 100.0 | 12.0 | 29.8 | 67.7 |
| 1.45% | 100.0 | 10.8 | 27.2 | 67.3 |
| 0.71% | 100.0 | 4.2 | 22.3 | 67.6 |
| 0.48% | 100.0 | 2.2 | 13.8 | 64.9 |
| 0.24% | 100.0 | 4.2 | 2.9 | 49.3 |
| 0.16% | 100.0 | 14.2 | 2.8 | 24.6 |
| 0.12% | 100.0 | −5.3 | −1.5 | 40.1 |
| 0.09% | 100.0 | −3.7 | −50.4 | 48.8 |

### EXAMPLE 6

### Removal of Xylanase Inhibitors from Feed Samples Using Immobilized Xylanase Xy1A1A

| | Buffer: | | | |
|---|---|---|---|---|
| % | 1xSAB | 1xSABWXIA | 1xSABWXIB | 1xSABWXIC |
| Waxy | % Residual Activity | | | |
| 2.86% | 100.0 | 2.7 | 12.4 | 45.1 |
| 1.45% | 100.0 | 6.4 | 13.5 | 43.8 |
| 0.71% | 100.0 | 2.7 | 9.5 | 39.6 |
| 0.48% | 100.0 | 1.5 | 3.8 | 36.0 |
| 0.24% | 100.0 | 1.6 | 1.5 | 27.2 |
| 0.16% | 100.0 | 1.9 | 1.6 | 18.6 |
| 0.12% | 100.0 | −0.1 | −2.6 | 18.1 |
| 0.09% | 100.0 | −0.5 | −14.6 | −0.8 |

### Preparation of Wheat Extract for Purification of Xylanase Inhibitors

Soissons wheat flour was ground in a KTec kitchen mill to pass through a 1 mm screen (USA Standard Test Screen #18). Approximately fifty grams of flour was resuspended in 500 mL of 100 mM sodium acetate buffer pH5.3 with 0.02% w/v sodium azide and stirred for 1 hour at room temperature. The slurry was centrifuged for 10 minutes at 5,000 rpm in a GS3 rotor at room temperature. The supernatant (WE) was collected and stored at 4° C. until used.

### Preparation of the Xylanase Affinity Column

Lyophilized xylanase, approximately 10 mg of Pichia pastoris produced Xy1A1A (rXy1A1A, lot Xv1-Xy1A1A-PB206), was resuspended in 1.25 mL distilled water and brought up to 5 mL with 0.1M NaHCO3 pH8.3. This solution was dialyzed against 4 L of 0.1M NaHCO3 for 5.5 hr at 4° C. and then added to distilled water-washed affigel-10. The xylanase-coupled affigel-10 was poured into a 2 mL column.

### Purification of Xylanase Inhibitor

The xylanase affinity column was first pre-eluted with 1 ml of 50% ethylene glycol, pH 11.5 and then washed with phosphate buffered saline, pH7.3 (PBS). The column was then pre-eluted with 0.1M glycine-HCl pH2.5 followed by equilibration in 6 ml of PBS.

Thirty mls of Soissons wheat extract was applied to the column by gravity. The column was then washed with PBS until no additional protein was eluted as monitored by absorbance at 280 nm. 35 ml of this flow through was collected and labeled wheat flow through (WFT). Proteins bound to the xylanase affinity column were eluted using 1 ml of 50% ethylene glycol pH 11.5 followed by PBS. Absorbance at 280 nm was recorded for the fraction. A total of 35 mL was collected and labeled WXI11.5. The WXI11.5 and WFT samples were dialyzed extensively against 1×SAB with a 3 kDa cut-off membrane.

After dialysis, protein concentrations were determined for WE, WFT, and WXI11.5 using the BCA™ method in a microtiter plate format and used to calculate the amount of protein per assay.

### Determination of the Xylanase Activity

Enzymatic activity was determined using wheat arabinoxylan as substrate and measuring the release of reducing ends by reaction of the reducing ends with either 3,5-dinitrosalicylic acid (DNS). The substrate was prepared as a 1.4% w/w solution of wheat arabinoxylan in 1×SAB. The DNS reagent consisted of 0.5% w/w, 15% sodium potassium tartrate, and 1.6% w/w sodium hydroxide. To perform the assay, five hundred microliters of substrate were combined with 200 microliters of each sample. After incubation at the desired temperature for the desired length of time, 700 microliters of DNS reagent was added. The contents were mixed and placed at 100° C. for 10 minutes. The contents were allowed to cool and then transferred to cuvettes and the absorbance at 540 nm was measured relative to known concentrations of xylose. The choice of enzyme dilution, incubation time, and incubation temperature could be varied by one skilled in the art.

TABLE 7

| | % Residual |
|---|---|
| Sample Name | Activity |
| Pichia pastoris Produced XylA1A | 100.0 |
| Pichia pastoris Produced XylA1A + Wheat | 28.4 |
| Extract | |
| Pichia pastoris Produced XylA1A + Wheat | 73.2 |
| Flow Through | |
| Pichia pastoris Produced XylA1A + AmSo₄ | 17.2 |
| ppt.d WXI pH1 1.5 | |

A xylanase sample, Pichia pastoris produced Xy1A1A, was diluted to ˜1:10000 into 100 mM Sodium Acetate buffer pH5.30, WE in 100 mM sodium acetate buffer pH5.30 at a concentration of 190 μg/ml, WFT in 100 mM sodium acetate buffer pH5.30 at a concentration of 134 μg/ml, and WXI11.5 in 100 mM sodium acetate buffer pH5.30 at a concentration of 0.58 μg/ml.

The P. pastoris produced Xy1A1A activity was reduced with the addition of the wheat extract to the sample. The wheat extract reduced the activity by 71.6 percent (From 4355 U/mg to 1238 U/mg). However, when the WFT was assayed, 73.2% of the xylanase activity was recovered. This indicates that the xylanase affinity column effectively removed 93.6% of the xylanase inhibitory activity present in the WE. When the purified wheat xylanase inhibitors (WXI11.5) was added to the Pichia pastoris produced Xy1A1A, the activity was reduced 82.8 percent (From 4355 U/mg to 747 U/mg) corresponding to 80.3% of the inhibitory activity present in the WE. This demonstrates that the majority of the xylanase inhibitors present in WE could be captured on this affinity resin.

### EXAMPLE 9

### Demonstration that the Addition of Xy1A1A_E79A Protein can be used to Recover Xylanase Activity in the Presence of Wheat Xylanase Inhibitors

### Preparation of Wheat Extract for Purification of Xylanase Inhibitors

Soissons wheat flour was ground in a KTec kitchen mill to pass through a 1 mm screen (USA Standard Test Screen #18). Approximately fifty grams of flour was resuspended in 500 mL of 100 mM sodium acetate buffer pH5.3 (abbrev. 1×SABWOA) and stirred for 1 hour at room temperature. The slurry was centrifuged for 10 minutes at 5,000 rpm in a GS3 rotor at room temperature. The supernatant was collected and stored at 4° C. until used.

### Preparation of the Xylanase Affinity Column

Lyophilized xylanase, approximately 10 mg of Pichia pastoris produced Xy1A1A, was resuspended in 1.25 mL distilled water and brought up to 5 mL with 0.1M NaHCO3 pH8.3. This solution was dialyzed against 4 L of 0.1M NaHCO3 for 5.5 hr at 4° C. and then added to distilled water-washed affigel-10. The xylanase-coupled affigel-10 was poured into a 2 mL column.

### Purification of Xylanase Inhibitor

The xylanase affinity column was pre-eluted with 1 ml of 0.1M glycine-HCl pH2.5 followed by equilibration in phosphate buffered saline, pH7.3 (PBS). Fifty mL of Soissons wheat extract was applied to the column by gravity. The column was then washed with PBS until no further protein was eluted as monitored by absorbance at 280 nm. Proteins bound to the xylanase affinity column were eluted using 1 ml of 0.1M glycine-HCl pH2.5 followed by 6 ml of PBS. Two ml fractions were collected. (repeated 10 times) Absorbance at 280 nm was recorded for each fraction. The fractions containing protein based on A280 were combined (˜22 mL) and dialyzed extensively against 1×SABWOA with a 3 kDa cut-off membrane. The dialyzed sample was labelled Wheat Xylanase Inhibitor (WXI).

### Preparation of Xylanase Assay Samples

The following xylanases samples were used: Pichia pastoris produced Xy1A1A and Avizyme 1310. These xylanase samples were diluted in 100 mM Sodium Acetate buffer pH5.30, AmSO₄ ppt.d WXI pH11.5 in 100 mM sodium acetate buffer pH5.30 at a concentration of 0.58 μg/ml, and Pichia pastoris produced Xy1A1A_E79A in 100 mM sodium acetate buffer pH5.30 at a concentration of 100 μg/ml.

### Determination of the Xylanase Activity

Enzymatic activity was determined using wheat arabinoxylan as substrate and measuring the release of reducing ends by reaction of the reducing ends with either 3,5-dinitrosalicylic acid (DNS). The substrate was prepared as a 1.4% w/w solution of wheat arabinoxylan (Megazyme P-WAXYM) in 100 mM sodium acetate buffer pH5.30. The DNS reagent consisted of 0.5% w/w, 15% sodium potassium tartrate, and 1.6% w/w sodium hydroxide. To perform the assay, five hundred microliters of substrate were combined with 200 microliters of the each sample. After incubation at the desired temperature for the desired length of time, 700 microliters of DNS reagent was added. The contents were mixed and placed at 100° C. for 10 minutes. The contents were allowed to cool and then transferred to cuvettes and the absorbance at 540 nm was measured relative to known concentrations of xylose. The choice of enzyme dilution, incubation time, and incubation temperature could be varied by one skilled in the art.

TABLE 8

| | | | Xylanase | |
|---|---|---|---|---|
| Active | Inactive | | Activity | % |
| Xylanase | Xylanase | | (umol/ | Residual |
| Proteins | Proteins | Inhibitor | min/mg) | Activity |
| | XylA1A_E79A | | 0.0 | 0.0 |
| | | WXI pH1 1.5 | 0.0 | 0.0 |
| | XylA1A-E79A | WXI pH1 1.5 | 0.0 | 0.0 |
| XylA1A | | | 4596 | 100.0 |
| XylA1A | XylA1A_E79A | | 4454 | 96.9 |
| XylA1A | | WXI pH1 1.5 | 754 | 16.4 |
| XylA1A | XylA1A_E79A | WXI pH1 1.5 | 4417 | 96.1 |
| Avizyme | | | 5659 | 100.0 |
| 1310 | | | | |
| Avizyme | XylA1A_E79A | | 7016 | 124.0 |
| 1310 | | | | |
| Avizyme | | WXI pH1 1.5 | 113 | 2.0 |
| 1310 | | | | |
| Avizyme | XylA1A_E79A | WXI pH1 1.5 | 6875 | 121.4 |
| 1310 | | | | |

No xylanase activity was detected in the samples containing only WXI11.5 and Xy1A1A_E79A. The combination of these two samples also displayed no xylanase activity. The addition of Pichia pastoris produced Xy1A1A_E79A to the two xylanase samples resulted in a slight increase in activity for Avizyme 1310 (from 5659 U/mg to 7016 U/mg) and a slight decrease in activity for the Pichia pastoris produced Xy1A1A (From 4596 U/mg to 4454 U/mg). The activities of the Pichia pastoris produced Xy1A1A and Avizyme 1310 xylanases were reduced to by 84% and 98%, respectively in the presence of WXI11.5. The addition of the Pichia pastoris produced Xy1A1A_E79A to these two xylanase samples in the presence of WXI11.5 increased the activities to 98 and 99% of the uninhibited levels for Avizyme 1310 and Pichia pastoris produced Xy1A1A, respectively. This demonstrates that the addition of E79A can effectively sequester inhibitors and allow nearly 100% recovery of xylanase activity in the presence of these inhibitors.

### EXAMPLE 10

### Determination of Extractable Xylanase Enzymatic Activity from Feed Stuffs at pH 5.3 by Reducing Sugar Assay

The assay is based on the detection of reducing ends released from wheat arabinoxylan (WAXY) substrate by the hydrolytic enzymatic action of xylanase. Substrate and enzyme are incubated for 240 minutes at 37 degrees centigrade, followed by simultaneous reaction quenching and colorimetric detection. Color formation, which is measured spectrophotometrically at 540 nm, is the result of reaction with DNS reagent with reducing sugars under alkaline conditions.

### Reagents

Wheat Arabinoxylan

0.4 M Sodium Hydroxide

DNS Reagent: Dissolve 5.0 g 3,5-dinitrosalicylic acid and 150 g sodium potassium tartrate tetrahydrate in 900 ml of 0.4 M Sodium Hydroxide. Transfer to a 1 L volumetric flask and adjust volume to 1 L with 0.4 M Sodium Hydroxide. Filter through 0.2 mm filter.

Sodium Acetate Buffer: 200 mM, pH 5.3 (2×SABWOA): Sodium azide should not be included in buffers for Quantum Xylanase in Feed Assays—this will interfere with the Quantum Xylanase Additive.

Sodium Acetate Buffer, 100 mM, pH 5.3 (1×SABWOA).

1.40% w/v wheat arabinoxylan in 1×SABWOA (Substrate Solution): Weigh 1.40 g wheat arabinoxylan into a 120 ml dry pyrex beaker. Wet the sample with 8.0 mL of 95% ethanol. Add 50. mL of 2×SABWOA and 30 mL of water. Cover with aluminum foil and place the slurry on a magnetic stirrer plate with vigorous stirring overnight or until dissolved. Transfer to a 100 mL volumetric flask. Wash the beaker with ˜10 mL water and combine with contents of volumetric flask. Adjust volume to 100 mL with water.

Xylose Stock Solution, 1.00 mg/mL D (+) Xylose in 1×SABWOA: Dissolve 50.0±0.5 mg D (+) xylose in 40 mL 1×SABWOA in a 50 mL glass beaker with stirring. Transfer solution to 50 mL volumetric flask. Wash beaker with ˜5 mL 1×SABWOA and combine in volumetric flask. Adjust volume to 50 mL with 1×SABWOA.

### Sample Extraction and Dilution Preparation

Feed Extraction: Add approximately 5.00 g±0.05 g of feed sample to a 50 mL volumetric flask. Record the mass of the added feed. Add 50 mL of 1×SABWOA to the flask and feed sample. Record the mass of buffer added. Repeat for all samples. Incubate samples at room temperature for 60 minutes with vigorous stirring (800-1000 rpm). The solution will attain a milky, cloudy appearance. Following the extraction, transfer ˜10 mL of the enzyme sample from the flasks to 16×100 mm glass tubes. Place the tubes into a centrifuge and centrifuge for 10 minutes at 1,000 g and room temperature (20-25° C.). Transfer ˜5 mL of the supernatant containing extracted xylanase enzyme to a fresh 16×100 mm glass tube. At least three replicates should be conducted for each feed sample being analyzed.

### Primary Dilution of Extract

Measure and record the mass of a 16×100 mm glass tube on a tared balance. One tube will be required for each sample extracted. Add 1.0 mL of the section 6.1 (a) extract. Record the mass of added extract. Add 4.0 mL of 1×SABWOA. Record the weight of buffer and extract. Vortex the samples for 1-2 minutes to mix.

Calculation of the Primary Dilution Factor: Take the mass of the added extract (approximately 1.0 g) and divide it by the total mass of liquid in the tube (approximately 5.0 g). The inverse of this value is the primary dilution factor. It will be approximately 5 based upon mass.

Assay Working Dilution: As varying xylanase concentrations will be encountered during the course of this assay, a rapid range finder study may be required to determine the optimal dilution rate to get a particular sample analysis onto scale. The range finder study is conducted by preparing the Primary Dilution of feed extract containing the xylanase enzyme as described above. Variations to the extraction method are then made with regards to the preparation of the working dilution listed below.

For the range finder study, a set of working dilutions of the extracted xylanase enzyme is made on a volumetric basis, and these are then run through a modified xylanase assay. The range finder assay may be run with only a single reaction tube for each dilution to be tested. Once the optimal dilution rate has been determined, prepare working dilutions according to the protocol detailed below. The target absorbance at 540 nm is between 0.4 and 1.2. As a rule-of-thumb, the assay working dilution can be calculated from the expected inclusion (in units of DNS U/kg) by dividing by 100. Thus, an enzyme sample that should have 1600 DNS U/kg would be diluted an additional 1:3.2 following the Primary Dilution. Note that samples having less than 500 DNS U/kg should still be diluted 1:5 to produce a background absorbance that is below an absorbance of 0.4.

On a tared balance, weigh and record the mass of a 16×100 mm glass test tube. Add and record the appropriate mass of the Primary Dilution as determined in the range finding study or using the rule-of-thumb calculation is needed to give ˜5 mL of Working Dilution. Add the appropriate mass of 1×SABWOA to obtain ˜5 mL of Assay Working Dilution. Record the mass of the added buffer. Finally, measure and record the mass of the test tube containing both extract and buffer on a tarred balance.

Calculation of the working dilution: Take the mass of the Primary Dilution and divide that value by the total mass of liquid in the test tube. The inverse of that number will be the working dilution factor.

Preparation of Xylose Standard Samples: Use the 1.00 mg/mL xylose solution (§5.8) to make up the following xylose standards:

TABLE 9

| | |
|---|---|
| Xylose Standard | |
| Solutions | Stock solutions |

| | Xylose | Xylose Stock | 1x SABWOA |
|---|---|---|---|
| No. | [μmols] | Solution [μl] | with [μl] |
| 1 | 0.00 | 0 | 500 |
| 2 | 0.20 | 75 | 425 |
| 3 | 0.40 | 150 | 350 |
| 4 | 0.60 | 225 | 275 |
| 5 | 0.80 | 300 | 200 |
| 6 | 1.00 | 375 | 125 |
| 7 | 1.20 | 450 | 50 |
| 8 | 1.33 | 500 | 0 |

These solutions can also be made in larger volumes and should be made fresh daily.

Aliquot the 200 μL, of xylose standards 1-8 (listed in the table above) into 13×100 mm test tubes in duplicate.

Add 0.5 mL of Substrate Solution to each standard tube, vortex to mix, and let stand for 15 minutes.

Add 0.7 mL of DNS Reagent to each standard tube and vortex to mix. Upon the addition of all of the reagents the final volume of each standard curve sample will be 1.4 mL. A xylose standard curve must be prepared each time a set of assays is performed. The concentration range of the xylose standard curve is such that standard 8 will produce an absorbance of approximately 1.2 at 540 nm. Assay sample absorbances should not go above this higher limit value. If so, dilute the test enzyme samples further and repeat the assay.

### Xylanase Enzyme Assay

Aliquot 0.5 mL of Substrate Solution into 13×100 mm glass test tubes and pre-incubate for 10 minutes at 37° C. (see summary of sample/reagent additions below). Prepare three test tubes for the enzyme reactions and one for the reaction blanks for each enzyme sample (4 substrate tubes total per sample).

Pre-incubate ˜5 mL working dilution enzyme samples for 10 minutes at 37° C. These incubation periods equilibrate substrate and enzyme to the temperature prior to reaction initiation.

Add 0.7 mL of DNS Reagent to the first of the four 0.5 mL substrate tubes. Vortex and return to the water bath.

Following the 10 minutes of pre-incubation, add 0.2 mL of working dilution enzyme sample to the first of the four 0.5 mL substrate tubes. Continue adding diluted enzyme sample to the additional substrate tubes at a constant rate (i.e., addition of diluted enzyme to a tube every 5 seconds). The constant enzyme addition rate established during this portion of the assay will be required again during the reaction quenching protocol. Subsequent to the addition of the last aliquot of diluted enzyme vortex all reaction tubes and return tubes 2-4 to the 37° C. water bath. Incubate for 240 minutes. Place the reaction blank tube in a rack at room temperature.

Following the 240 minute incubation period, add 0.7 mL of color stop solution to each enzyme reaction test tube using the constant sample addition rate established above. The use of a constant addition rate will ensure that each sample undergoes the same reaction time. Vortex to mix all quenched test tubes.

### Summary of Sample/Reagent Additions

Enzyme Reaction Samples

0.5 mL substrate (pre-incubate 10 minutes, 3 tubes per enzyme replicate) diluted xylanase samples (pre-incubate 10 minutes, 5 mL sample volume recommended)

0.2 mL of diluted enzyme added to substrate tubes, incubate for 240 minutes at 37° C.

0.7 mL color stop solution (added following 240 minutes of incubation)

### Enzyme Sample Blanks

0.5 mL substrate (pre-incubate 10 minutes, 1 tube per enzyme replicate)

0.7 mL color stop solution (added following 10 minutes of pre-incubation)

0.2 mL of diluted enzyme added

### Xylose Standard Curve Samples

0.2 mL of each xylose standard (see table above)

0.5 mL of substrate, mix and let stand for 240 minutes at room temperature

0.7 mL of color stop solution added after 240 minutes

### Spectroscopic Measurements and Enzyme Activity Calculations

Using a plastic cuvette (1 cm path length, semi-micro), zero the spectrophotometer at 540 nm using water. Read all reaction, blank, and xylose standard curve samples at 540 nm and record values.

For the 7 xylose standard curve samples take each absorbance measurement and subtract the 0 μmol xylose reading (xylose standard 1). This corrects all of the xylose standard curve readings by subtracting a reagent blank.

Plot the absorbance at 540 nm as a function of xylose amount, and then calculate the “best fit” line through the data set using a linear regression program. For the enzyme reaction samples, take the average of the three 240 minute readings (these should be within 5% of one another and ideally fall into the absorbance range of the xylose standards) and subtract the background (zero minute) reading.

Take the background corrected absorbance for each replicate and interpolate using the xylose standard curve regression parameters. The interpolated value is calculated in units of μmols.

Divide each interpolated μmols value by 240 minutes for the time of reaction and by the mass (in grams) of a 0.2 mL aliquot of 1×SABWOA. The units of this calculation are in μmols/min/g or in xylanase units per gram of diluted extract (XU/g) by definition.

Take the XU/g value and multiply it by the dilution factor used to get the sample readings on scale. The dilution factor is the product of the primary and assay working dilutions.

Multiply the dilution adjusted XU/g by the total mass of buffer that was used in the xylanase extraction procedure, and then divide that value by the amount of feed used in the extraction. The final calculated activity is a mass based activity that is represented in xylanase units per kilogram of feed.

### EXAMPLE 11

### Extraction of Xylanase Enzymatic Activity From Feed by Buffer Containing XyA1A1_E79A Inactive Xylanase Molecule Followed by Determination of Extractable Xylanase Enzymatic Activity at pH 5.3 by Reducing Sugar Assay

The assay is based on the detection of reducing ends released from wheat arabinoxylan (WAXY) substrate by the hydrolytic enzymatic action of xylanase. Subtrate and enzyme are incubated for 240 minutes at 37 degrees centigrade, followed by simultaneous reaction quenching and colorimetric detection. Color formation, which is measured spectrophotometrically at 540 nm, is the result of reaction with DNS reagent with reducing sugars under alkaline conditions. The present invention utilizes XyA1A_E79A inactive xylanase molecule in the extraction buffer, thereby enhances the recovery of xylanase enzymes contained in feed samples.

### Reagents

XyA1A1_E79A

Wheat Arabinoxylan

0.4 M Sodium Hydroxide

DNS Reagent: Dissolve 5.0 g 3,5-dinitrosalicylic acid and 150. g sodium potassium tartrate tetrahydrate in 900 ml of 0.4 M Sodium Hydroxide. Transfer to a 1 L volumetric flask and adjust volume to 1 L with 0.4 M Sodium Hydroxide. Filter through 0.2 mm filter.

Sodium Acetate Buffer: 200 mM, pH 5.3 (2×SABWOA): Sodium azide should not be included in buffers for Quantum Xylanase in Feed Assays, because it interferes with the Quantum Xylanase Additive.

Sodium Acetate Buffer, 100 mM, pH 5.3 (1×SABWOA).

Sodium Acetate Buffer, 100 mM, pH 5.3 with XyA1A1_E79A (1×SABWOA with E79A): Add 1.00 g of XyA1A1_E79A to a 1000 mL volumetric flask. Add 500 mL 2×SABWOA to a 500 mL volumetric flask. Transfer to the 1000 mL volumetric flask containing XyA1A1_E79A. Wash 500 mL volumetric flask with water and combine in 1000 mL flask with 2×SABWOA and E79A. Adjust volume to 1000 mL with water. Stir until all XyA1A1_E79A is dissolved.

1.40% w/v wheat arabinoxylan in 1×SABWOA (Substrate Solution): Accurately weigh 1.40 g wheat arabinoxylan into a 120 ml dry pyrex beaker. Wet the sample with 8.0 mL of 95% ethanol. Add 50 mL of 2×SABWOA and 30 mL of water. Cover with aluminum foil and place the slurry on a magnetic stirrer plate with vigorous stirring overnight or until dissolved. Transfer to a 100 mL volumetric flask. Wash the beaker with ˜10 mL water and combine with contents of volumetric flask. Adjust volume to 100 mL with water.

Xylose Stock Solution, 1.00 mg/mL D (+) Xylose in 1×SABWOA: Dissolve 50.0±0.5 mg D (+) xylose in 40 mL 1×SABWOA with E79A in a 50 mL glass beaker with stirring. Transfer solution to 50 mL volumetric flask. Wash beaker with ˜5 mL 1×SABWOA with E79A and combine in volumetric flask. Adjust volume to 50 mL with 1×SABWOA with E79A.

### Sample Extraction and Dilution Preparation

Feed Extraction: On a tared balance measure and record the mass of an empty 50 mL volumetric flask. Add approximately 5.00 g±0.05 g of feed sample. Record the mass of the added feed. Tare the flask and feed. Add 50 mL of 1×SABWOA with E79A to the flask and feed sample. Record the mass of buffer added. Repeat for all samples. Incubate samples at room temperature for 60 minutes with vigorous stirring (800-1000 rpm). The solution will attain a milky, cloudy appearance. Following the extraction, transfer ˜10 mL of the enzyme sample from the flasks to 16×100 mm glass tubes. Place the tubes into a centrifuge and centrifuge for 10 minutes at 1,000 g and room temperature (20-25° C.). Transfer ˜5 mL of the supernatant containing extracted xylanase enzyme to a fresh 16×100 mm glass tube. At least three replicates should be conducted for each feed sample being analyzed.

### Primary Dilution of Extract

Measure and record the mass of a 16×100 mm glass tube on a tared balance. One tube will be required for each sample extracted. Tare the balance with the empty tube on it, and then add 1.0 mL of the section 6.1 (a) extract. Record the mass of added extract. Add 4.0 mL of 1×SABWOA with E79A. Record the weight of buffer and extract. Vortex the samples for 1-2 minutes to mix.

Calculation of the Primary Dilution Factor: Take the mass of the added extract (approximately 1.0 g) and divide it by the total mass of liquid in the tube (approximately 5.0 g). The inverse of this value is the primary dilution factor. It will be approximately 5 based upon mass.

Assay Working Dilution: As XyA1A1_E79A at varying xylanase concentrations will be encountered during the course of this assay, a rapid range finder study may be required to determine the optimal dilution rate to get a particular sample analysis onto scale. The range finder study is conducted by preparing the Primary Dilution of feed extract containing the xylanase enzyme as described above. Variations to the extraction method are then made with regards to the preparation of the working dilution listed below.

For the range finder study, a set of working dilutions of the extracted xylanase enzyme is made on a volumetric basis, and these are then run through a modified XyA1A_E79A assay. The range finder assay may be run with only a single reaction tube for each dilution to be tested. Once the optimal dilution rate has been determined, prepare working dilutions according to the protocol detailed below. The target absorbance at 540 nm is between 0.4 and 1.2. As a rule-of-thumb, the assay working dilution can be calculated from the expected inclusion (in units of DNS U/kg) by dividing by 100. Thus, an enzyme sample that should have 1600 DNS U/kg would be diluted an additional 1:3.2 following the Primary Dilution. Note that samples having less than 500 DNS U/kg should still be diluted 1:5 to produce a background absorbance that is below an absorbance of 0.4.

Weigh and record the mass of a 16×100 mm glass test tube. Tare the balance, then add and record the appropriate mass of the Primary Dilution as determined in the range finding study or using the rule-of-thumb calculation is needed to give ˜5 mL of Working Dilution. Add the appropriate mass of 1×SABWOA with E79A to obtain ˜5 mL of Assay Working Dilution. Record the mass of the added buffer. Finally, measure and record the mass of the test tube containing both extract and buffer.

Calculation of the working dilution: Take the mass of the Primary Dilution and divide that value by the total mass of liquid in the test tube. The inverse of that number will be the working dilution factor.

Preparation of Xylose Standard Samples: Use the 1.00 mg/mL xylose solution (§5.8) to make up the following xylose standards:

TABLE 10

| | |
|---|---|
| Xylose Standard | |
| Solutions | Stock solutions |

| | Xylose | Xylose Stock | 1x SABWOA |
|---|---|---|---|
| No. | [μmols] | Solution [μl] | with E79A [μl] |
| 1 | 0.00 | 0 | 500 |
| 2 | 0.20 | 75 | 425 |
| 3 | 0.40 | 150 | 350 |
| 4 | 0.60 | 225 | 275 |
| 5 | 0.80 | 300 | 200 |
| 6 | 1.00 | 375 | 125 |
| 7 | 1.20 | 450 | 50 |
| 8 | 1.33 | 500 | 0 |

These solutions can also be made in larger volumes and should be made fresh daily.

Aliquot the 200 μL, of xylose standards 1-8 (listed in the table above) into 13×100 mm test tubes in duplicate.

Add 0.5 mL of Substrate Solution to each standard tube, vortex to mix, and let stand for 15 minutes.

Add 0.7 mL of DNS Reagent to each standard tube and vortex to mix. Upon the addition of all of the reagents the final volume of each standard curve sample will be 1.4 mL. A xylose standard curve must be prepared each time a set of assays is performed. The concentration range of the xylose standard curve is such that standard 8 will produce an absorbance of approximately 1.2 at 540 nm. Assay sample absorbances should not go above this higher limit value. If so, dilute the test enzyme samples further and repeat the assay.

### Xylanase Enzyme Assay

Aliquot 0.5 mL of Substrate Solution into 13×100 mm glass test tubes and pre-incubate for 10 minutes at 37° C. (see summary of sample/reagent additions below). Prepare three test tubes for the enzyme reactions and one for the reaction blanks for each enzyme sample (4 substrate tubes total per sample).

Pre-incubate ˜5 mL working dilution enzyme samples for 10 minutes at 37° C. These incubation periods equilibrate substrate and enzyme to the temperature prior to reaction initiation.

Add 0.7 mL of DNS Reagent to the first of the four 0.5 mL substrate tubes. Vortex and return to the water bath.

Following the 10 minutes of pre-incubation, add 0.2 mL of working dilution enzyme sample to the first of the four 0.5 mL substrate tubes. Start a timer upon the addition of diluted enzyme to the first tube. Continue adding diluted enzyme sample to the additional substrate tubes at a constant rate (i.e., addition of diluted enzyme to a tube every 5 seconds). The constant enzyme addition rate established during this portion of the assay will be required again during the reaction quenching protocol. Subsequent to the addition of the last aliquot of diluted enzyme vortex all reaction tubes and return tubes 2-4 to the 37° C. water bath. Incubate for 240 minutes. Place the reaction blank tube in a rack at room temperature.

Following the 240 minute incubation period, add 0.7 mL of color stop solution to each enzyme reaction test tube using the constant sample addition rate established above. The use of a constant addition rate will ensure that each sample undergoes the same reaction time. Vortex to mix all quenched test tubes.

### Summary of Sample/Reagent Additions

Enzyme Reaction Samples

0.5 mL substrate (pre-incubate 10 minutes, 3 tubes per enzyme replicate) diluted xylanase samples (pre-incubate 10 minutes, 5 mL sample volume recommended)

0.2 mL of diluted enzyme added to substrate tubes, incubate for 240 minutes at 37° C.

0.7 mL color stop solution (added following 240 minutes of incubation)

### Enzyme Sample Blanks

0.5 mL substrate (pre-incubate 10 minutes, 1 tube per enzyme replicate)

0.7 mL color stop solution (added following 10 minutes of pre-incubation)

0.2 mL of diluted enzyme added

### Xylose Standard Curve Samples

0.2 mL of each xylose standard (see table above)

0.5 mL of substrate, mix and let stand for 240 minutes at room temperature

0.7 mL of color stop solution added after 240 minutes

### Spectroscopic Measurements and Enzyme Activity Calculations

Using a plastic cuvette (1 cm path length, semi-micro), zero the spectrophotometer at 540 nm using water. Read all reaction, blank, and xylose standard curve samples at 540 nm and record values.

For the 7 xylose standard curve samples take each absorbance measurement and subtract the 0 μmol xylose reading (xylose standard 1). This corrects all of the xylose standard curve readings by subtracting a reagent blank.

Plot the absorbance at 540 nm as a function of xylose amount, and then calculate the “best fit” line through the data set using a linear regression program. For the enzyme reaction samples, take the average of the three 240 minute readings (these should be within 5% of one another and ideally fall into the absorbance range of the xylose standards) and subtract the background (zero minute) reading.

Take the background corrected absorbance for each replicate and interpolate using the xylose standard curve regression parameters. The interpolated value is calculated in units of μmols.

Divide each interpolated μmols value by 240 minutes for the time of reaction and by the mass (in grams) of a 0.2 mL aliquot of 1×SABWOA with E79A. The units of this calculation are in μmols/min/g or in xylanase units per gram of diluted extract (XU/g) by definition.

Take the XU/g value and multiply it by the dilution factor used to get the sample readings on scale. The dilution factor is the product of the primary and assay working dilutions.

Multiply the dilution adjusted XU/g by the total mass of buffer that was used in the xylanase extraction procedure, and then divide that value by the amount of feed used in the extraction. The final calculated activity is a mass based activity that is represented in xylanase units per kilogram of feed.

### EXAMPLE 12

### Increasing the Recovery of Xylanase from Mash and Pelleted Feed Samples By Using Xy1A1A_E79A

### Preparation of Feed Samples

Wheat-based broiler diets were prepared by mixing the components shown in Table X. Three separate diet batches were prepared: starter, grower and finisher diets. xylanase enzyme was dosed into each diet at various levels as shown in Table XI, thus generating a series of sub-batches of each mash feed dosed with different levels of xylanase enzyme. Samples were taken from the each sub-batch for analysis of enzyme activity.

To prepare pelleted feed samples, the sub-batches of mash feed were passed through a pellet mill. The mill was operated with maximum temperature setting of 75° C., the average temperature of the die face set during manufacture of the pellets was 68.0±0.8° C.

TABLE 11
Diet Composition (g/kg)

| Component | Starter | Grower/Finisher |
|---|---|---|
| Wheat | 552.4 | 544.5 |
| Rye | 50.0 | 70.0 |
| Soybean (heat treated) | 75.0 | 100.0 |
| Soybean meal hipro | 185.0 | 150.0 |
| Corn gluten meal (580 cp.) | 25.0 | 25.0 |
| Potato protein | 10.0 | 10.0 |
| Fishmeal (700 cp) | 15.0 | — |
| Soy oil | 14.5 | 13.0 |
| Blended animal fat | 35.0 | 55.0 |
| Premix | 5.0 | 5.0 |
| Limestone | 14.0 | 11.5 |
| Monocalcium phosphate | 11.5 | 7.0 |
| Sodium chloride | 1.8 | 1.8 |
| Sodium bicarbonate | 2.0 | 2.1 |
| L-lysine HCl | 1.8 | 2.6 |
| DL-methionine | 1.6 | 1.8 |
| L-threonine | 0.4 | 0.7 |
| Total | 1000.0 | 1000.0 |

All diets contained the coccidiostatic Clinacox ®

TABLE 12
Dose Levels of Xylanase enzyme

| Diet # | Xylanase Level (IU/g) |
|---|---|
| 1 | 0 |
| 2 | 0.4 |
| 3 | 0.8 |
| 4 | 1.6 |
| 5 | 3.2 |
| 6 | 32 |

Xylanase enzyme was dosed at these levels into starter, grower and finisher diets.

The feed samples were extracted and assayed by the methods detailed in Example 10 (extraction & assay without XyA1A_E79A) and Example 11 (extraction & assay with XyA1A1_E79A) in order to compare the effect of including the inactive xylanase on yield of extracted enzyme.

### Results

Table 13 presents the results from extracting xylanase enzyme from mash feed with or without the XyA1A_E79A protein (abbreviated E79A). Measured xylanase increased an average of 2.8-fold, an increase that was statistically significant (P<0.05).

Table 14 presents the results from extracting xylanase enzyme from pelleted feed with or without E79A protein. Measured xylanase increased an average of 2.9-fold, an increase that was statistically significant (P<0.05).

The combined data set of both mash and pelleted data showed an average increase in recovery of xylanase enzymatic activity of 2.9 fold that was statistically significant (P<0.0005). The average recovery of xylanase activity in sample extracted and assayed with E79A was 72.5% of the dosed level of xylanase protein.

The recovery when E79A was not present was only 18.8%. Thus XyA1A_E79A greatly increased the yield of xylanase from both mash and pelleted feed samples when it was included in extraction buffer and assay buffer.

TABLE 13
Effect of including E79A protein in extraction buffer on recovery of
xylanase activity from mash feed.

| Diet Type | | |
|---|---|---|
| Starter | Grower | Finisher |

| Xylanase | − | + | − | + | − | + |
|---|---|---|---|---|---|---|
| Dose (IU/g) | Measured Xylanase (IU/g) | | | | | |
| 0 | 0.29 | 0.55 | 0.32 | 0.64 | 0.28 | 0.61 |
| 0.4 | 0.35 | 0.78 | 0.4 | 0.86 | 0.43 | 0.85 |
| 0.8 | 0.4 | 1.05 | 0.46 | 1.15 | 0.32 | 1.25 |
| 1.6 | 0.54 | 1.74 | 0.58 | 2.25 | 0.64 | 2.24 |
| 3.2 | 0.81 | 2.59 | 0.82 | 3.64 | 0.76 | 3.08 |
| 32 | 9.95 | 22.8 | 12.25 | 24.62 | 9.3 | 20.68 |

− samples extracted & assayed with SABWOA without E79A
+ samples extracted & assayed with SABWOA with E79A

TABLE 14
Effect of including E79A protein in extraction buffer on recovery of
xylanase activity from pelleted feed.

| Diet Type | | |
|---|---|---|
| Starter | Grower | Finisher |

| Xylanase | − | + | − | + | − | + |
|---|---|---|---|---|---|---|
| Dose (IU/g) | Measured Xylanase (IU/g) | | | | | |
| 0 | 0.28 | 0.52 | 0.26 | 0.58 | 0.29 | 0.55 |
| 0.4 | 0.32 | 0.78 | 0.37 | 0.86 | 0.37 | 0.79 |
| 0.8 | 0.34 | 1.04 | 0.41 | 1.13 | 0.3 | 1.17 |
| 1.6 | 0.52 | 1.75 | 0.48 | 1.93 | 0.47 | 1.65 |
| 3.2 | 0.78 | 2.46 | 0.77 | 3.82 | 0.75 | 2.94 |
| 32 | 9.99 | 22.85 | 9.48 | 23.19 | 8.92 | 21.23 |

− samples extracted & assayed with SABWOA without E79A
+ samples extracted & assayed with SABWOA with E79A

What is claimed is:

1) A modified xylanase polypeptide, wherein the modification is at amino acid residue number 78 of the amino acid sequence depicted by SEQ ID NO. 3 or the equivalent position in other homologous xylanase polypeptides, wherein said modified xylanase polypeptide is inactive yet retains its ability to bind to xylanase inhibitors.

2) A modified xylanase polypeptide according to claim 1, wherein the modification is at amino acid residue number 78 of the amino acid sequence depicted by SEQ ID NO. 3 or equivalent position in a class 11 xylanase polypeptide.

3) A modified xylanase polypeptide according to claim 1, wherein the modification is at amino acid residue number 78 of amino acid sequence depicted by SEQ ID NO. 3 or equivalent position in a xylanase amino acid sequence depicted by SEQ ID NOS. 4 through 114.

4) An isolated nucleic acid molecule encoding the modified xylanase polypeptide of claim 1.

5) An isolated nucleic acid molecule encoding the modified xylanase polypeptide of claim 2.

6) An isolated nucleic acid molecule encoding the modified xylanase polypeptide of claim 3.

7) An expression cassette comprising a nucleic acid molecule according to claim 4 encoding an inactive xylanase protein.

8) A vector comprising at least one expression cassette of claim 7.

9) A recombinant host cell comprising the vector according to claim 8.

10) The recombinant host cell of claim 9, wherein the host cell is a bacteria, yeast or fungal cell.

11) The recombinant host cell of claim 10, wherein the bacteria, yeast or fungal cell is a Kluyveromyces, saccharomyces, Shizosaccharomyces, Trichosporon, Schwanniomyces, Pichia, Hansuela, Eschericia, Psudomonas, Lactobacillus, Bacillus, Aspergillus, Rhizopus, Trichoderma, Neurospora, Mucor, or Penicillium cell.

12) The recombinant host cell of claim 10, wherein the host cell is Pichia pastoris.

13) A modified xylanase enzyme having the amino acid sequence of SEQ ID NO. 2.

14) An isolated nucleic acid molecule encoding the modified xylanase enzyme of claim 13.

15) A method for recovering the activity of a xylanase enzyme, comprising the steps of:
a) providing an inactive xylanase molecule capable of binding to a xylanase inhibitor molecule;
b) mixing said inactive xylanase molecule with plant-derived material comprising a xylanase enzyme and a xylanase inhibitor under conditions sufficient for said inactive xylanase molecule and said xylanase inhibitor to bind together directly or indirectly;
c) measuring the activity of said xylanase enzyme.

16) A method for recovering the activity of a xylanase enzyme according to claim 15, wherein said inactive xylanase molecule comprises an inactive catalytic nucleophile.

17) A method for recovering the activity of a xylanase enzyme according to claim 16, wherein said inactive catalytic nucleophile corresponds to position 78 of SEQ ID NO. 3.

18) A method for recovering the activity of a xylanase enzyme according to claim 16, wherein said inactive catalytic nucleophile was made inactive by causing inactive amino acid substitutions in the active-site mutations.

19) A method for recovering the activity of a xylanase enzyme according to claim 15, wherein said xylanase molecule is selected from the group consisting of SEQ ID NO. 3 through 115.
